(19) United States
(12) Patent Application Publication
(10) Pub. No.: US 2023/0183806 A1
(43) Pub. Date: Jun. 15, 2023

(54) METHOD FOR DETECTING PARKINSON'S DISEASE

(71) Applicants: KAO CORPORATION, Chuo-ku, Tokyo (JP); JUNTENDO EDUCATIONAL FOUNDATION, Bunkyo-ku, Tokyo (JP)

(72) Inventors: Yuya UEHARA, Utsunomiya-shi, Tochigi (JP); Takayoshi INOUE, Koto-ku, Tokyo (JP); Nobutaka HATTORI, Bunkyo-ku, Tokyo (JP); Shinji SAIKI, Bunkyo-ku, Tokyo (JP); Shin-Ichi UENO, Bunkyo-ku, Tokyo (JP); Haruka TAKESHIGE, Bunkyo-ku, Tokyo (JP)

(73) Assignees: KAO CORPORATION, Chuo-ku, Tokyo (JP); JUNTENDO EDUCATIONAL FOUNDATION, Bunkyo-ku, Tokyo (JP)

(21) Appl. No.: 17/924,640

(22) PCT Filed: May 14, 2021
(86) PCT No.: PCT/JP2021/018511

§ 371 Date: Nov. 10, 2022

(30) Foreign Application Priority Data
May 14, 2020 (JP) 2020-085430

(51) Int. Cl. C12Q 1/6883

(52) U.S. Cl. CPC C12Q 1/6883; C12Q 1/6869

(57) ABSTRACT

Provided are a marker gene for detecting Parkinson's disease, and a method for detecting Parkinson's disease by using the marker gene. The method for detecting Parkinson's disease in a test subject comprises a step of measuring an expression level of at least one gene selected from the group of 4 genes consisting of SNORA16A, SNORA24, SNORA50 and REXO1L2P or an expression product thereof in a biological sample collected from the test subject.

### FIELD OF THE INVENTION

The present invention relates to a method for detecting Parkinson's disease by using a Parkinson's disease marker.

### BACKGROUND OF THE INVENTION

Parkinson's disease is pathologically a progressive neurodegenerative disease composed mainly of the formation of Lewy body having α-synuclein aggregates as a main component, the degeneration of dopaminergic neurons in the substantia nigra of the midbrain, and cell death, and is clinically a disease composed mainly of movement disorder such as muscle stiffness, tremor, hypokinesis, or gait disturbance.

Parkinson's disease is the second most common neurodegenerative disease after Alzheimer's disease. Its morbidity prevalence rate is 120 to 130 per 100,000 people, and it is estimated that there are approximately 140,000 patients in Japan.

At present, there exists no definitive therapy for Parkinson's disease. It is considered important for QOL maintenance to control symptoms by symptomatic therapy based on the supplementation of L-DOPA or the like.

However, subjective symptoms of movement disorder appear in an intermediate stage thereof or later. Thus, there is a demand for early diagnosis and early intervention of the disease.

For example, the detection of α-synuclein accumulation as well as the detection of microRNA derived from circulating serum (Patent Literature 1) and the measurement of the concentration ratio of tyrosine to phenylalanine in blood (Patent Literature 2) have been proposed as biomarkers for detecting Parkinson's disease. It has also been reported that: the formation of α-synuclein aggregates is observed in the skin, as in the brain, of Parkinson's disease patients (Non Patent Literature 1); and Parkinson's disease patients manifest skin diseases or symptoms such as seborrheic dermatitis, melanoma, bullous pemphigoid, or rosacea (Non Patent Literature 2). Although it is also considered that skin conditions are related in some way to Parkinson's disease, its scientific relation is totally unknown.

Meanwhile, techniques of examining current or future physiological states in vivo in humans by the analysis of nucleic acids such as DNA or RNA in biological samples have been developed. The analysis using nucleic acids has the advantages that: exhaustive analysis methods have already been established and abundant information can be obtained by one analysis; and the functional connection of analysis results is easily performed on the basis of many research reports on single-nucleotide polymorphism, RNA functions, and the like. Nucleic acids derived from a biological origin can be extracted from body fluids such as blood, secretions, tissues, and the like. It has recently been reported that: RNA contained in skin surface lipids (SSL) can be used as a biological sample for analysis; and marker genes of the epidermis, the sweat gland, the hair follicle and the sebaceous gland can be detected from SSL (Patent Literature 3).

(Patent Literature 1) JP-A-2019-506183

(Patent Literature 2) JP-A-2016-75644

(Patent Literature 3) WO 2018/008319

(Non Patent Literature 1) Rodriguez-Leyva I et al. Ann Clin Transl Neurol. 2014 (modified)

(Non Patent Literature 2) Ravn A H et al. Clin Cosmet Investig Dermatol. 2017

### SUMMARY OF THE INVENTION

The present invention relates to the following 1) to 3).

1) A method for detecting Parkinson's disease in a test subject, comprising a step of measuring an expression level of at least one gene selected from the group of 4 genes consisting of SNORA16A, SNORA24, SNORA50 and REXO1L2P or an expression product thereof in a biological sample collected from the test subject.

2) A test kit for detecting Parkinson's disease, the kit being used in a method according to 1), and comprising an oligonucleotide which specifically hybridizes to the gene, or an antibody which recognizes an expression product of the gene.

3) A marker for detecting Parkinson's disease comprising at least one gene selected from the groups of genes shown in Tables 3-1 to 3-4 and Tables 6-1 and 6-2 or an expression product thereof.

### BRIEF DESCRIPTION OF DRAWING

FIG. 1 shows confusion matrix in which predictive values in the optimum prediction model and actually measured values were plotted in test data.

### DETAILED DESCRIPTION OF THE INVENTION

The present invention relates to a provision of a marker for detecting Parkinson's disease and a method for detecting Parkinson's disease by using the marker.

The present inventors collected SSL from the skin of Parkinson's disease patients and healthy subjects and exhaustively analyzed the expression state of RNA contained in the SSL as sequence information, and consequently found that the expression levels of particular genes significantly differ therebetween and Parkinson's disease can be detected on the basis of this index.

The present invention enables Parkinson's disease to be conveniently and noninvasively detected in an early stage with high accuracy, sensitivity and specificity.

All patent literatures, non patent literatures, and other publications cited herein are incorporated herein by reference in their entirety.

In the present invention, the term “nucleic acid” or “polynucleotide” means DNA or RNA. The DNA includes all of cDNA, genomic DNA, and synthetic DNA. The “RNA” includes all of total RNA, mRNA, rRNA, tRNA, non-coding RNA and synthetic RNA.

In the present invention, the “gene” encompasses double-stranded DNA including human genomic DNA as well as single-stranded DNA including cDNA (positive strand), single-stranded DNA having a sequence complementary to the positive strand (complementary strand), and their fragments, and means matter containing some biological information in sequence information on bases constituting DNA.

The “gene” encompasses not only a “gene” represented by a particular nucleotide sequence but a nucleic acid encoding a congener (i.e., a homolog or an ortholog), a variant such as gene polymorphism, and a derivative thereof.

The names of genes disclosed herein follow Official Symbol described in NCBI ([www.ncbi.nlm.nih.gov/]). Meanwhile, gene ontology (GO) follows Pathway ID. described in String ([string-db.org/]).

In the present invention, the “expression product” of a gene conceptually encompasses a transcription product and a translation product of the gene. The “transcription product” is RNA resulting from the transcription of the gene (DNA), and the “translation product” means a protein which is encoded by the gene and translationally synthesized on the basis of the RNA.

In the present invention, the “Parkinson's disease” means an idiopathic and progressive disease which has the degeneration of dopaminergic neurons in the substantia nigra pars compacta as a main lesion and manifests three motor symptoms (tremor at rest, rigidity, and bradykinesia or akinesia) in a slowly progressive manner.

In the present invention, the “detection” of Parkinson's disease means to elucidate the presence or absence of Parkinson's disease and may be used interchangeably with the term “test”, “measurement”, “determination”, “evaluation” or “assistance of evaluation”. In the present specification, the term “determination” or “evaluation” does not include determination or evaluation by a physician.

The 4 genes consisting of SNORA16A, SNORA24, SNORA50 and REXO1L2P according to the present invention are genes selected from the 33 genes described in Table A given below for which the expression level of SSL-derived RNA was found to be significantly increased (UP) or decreased (DOWN) in Parkinson's disease patients compared with healthy subjects, as shown in Examples mentioned later. The 4 genes are genes whose relation to Parkinson's disease has previously been unknown (indicated by boldface in the table).

TABLE A

| | p value | p value | |
|---|---|---|---|
| Symbol | (Test 1) | (Test 2) | Regulation |
| ANKRD12 | 0.010769 | 0.022801 | DOWN |
| C10orf116 | 0.027915 | 0.039587 | UP |
| CCL3 | 0.008678 | 0.028353 | DOWN |
| CCNI | 0.004032 | 0.027019 | DOWN |
| CD83 | 0.041752 | 0.029159 | DOWN |
| CNFN | 0.024347 | 1.85E−05 | UP |
| CNN2 | 0.023711 | 0.045042 | DOWN |
| CSF2RB | 0.020573 | 0.047037 | DOWN |
| CXCR4 | 0.000244 | 0.020358 | DOWN |
| EGR2 | 0.005989 | 0.033983 | DOWN |
| EMP1 | 0.010452 | 0.000953 | UP |
| ITGAX | 0.027931 | 0.014582 | DOWN |
| KCNQ1OT1 | 0.0414 | 0.015544 | UP |
| LCE3D | 0.01773 | 0.000578 | UP |
| LITAF | 0.014086 | 0.02915 | DOWN |
| NDUFA4L2 | 0.047011 | 2.72E−05 | UP |
| NDUFS5 | 0.028286 | 0.011341 | UP |
| POLR2L | 0.005102 | 0.0376 | UP |
| REXO1L2P | 0.021096 | 0.016022 | UP |
| RHOA | 0.003152 | 0.004939 | DOWN |
| RNASEK | 0.030621 | 0.046581 | DOWN |
| RPL7A | 0.040024 | 0.003107 | UP |
| RPS26 | 0.020174 | 0.015282 | UP |
| SERINC1 | 0.046063 | 0.011959 | DOWN |
| SERP1 | 0.033858 | 0.027307 | DOWN |
| SERPINB4 | 0.048165 | 0.009405 | UP |
| SLC25A3 | 0.040817 | 0.031602 | UP |
| SNORA16A | 0.005217 | 3.37E−05 | UP |
| SNORA24 | 0.001017 | 0.00062 | UP |
| SNORA50 | 0.010607 | 0.004445 | UP |
| SNRPG | 0.002506 | 0.003904 | UP |
| SRRM2 | 0.036848 | 0.010131 | DOWN |
| UQCRH | 0.01058 | 0.030619 | UP |

33 genes shown in Table A were obtained by converting data (read count values) on the expression level of RNA extracted from SSL of test subjects of two tests (Test 1: 15 healthy subjects and 15 Parkinson's disease patients, Test 2: 50 healthy subjects and 50 Parkinson's disease patients) to RPM values which normalize the read count values for difference in the total number of reads among samples, identifying RNA (Test 1: 111 genes with increased expression and 68 genes with decreased expression (a total of 179 gene, Tables 1-1 to 1-5), Test 2: 565 genes with increased expression and 294 genes with decreased expression (a total of 859 gene, Tables 1-6 to 1-27) which attained a p value of 0.05 or less in Student's t-test in Parkinson's disease patients compared with healthy subjects on the basis of values obtained by the conversion of the RPM values to logarithmic values to base 2 (Log₂ RPM values), and selecting common genes with increased expression (18 genes) and genes with decreased expression (15 genes) between Test 1 and Test 2.

Thus, a gene selected from the group consisting of the 179 genes and the 859 genes (a total of 1,005 genes except for duplication) or an expression product thereof is capable of serving as a Parkinson's disease marker for detecting Parkinson's disease. Among them, a gene selected from the group consisting of 33 genes shown in Table A or an expression product thereof is a preferred Parkinson's disease marker.

In Table A and Table 1 mentioned later, the “p value” refers to the probability of observing extreme statistics based on statistics actually calculated from data under null hypothesis in a statistical test. Thus, a smaller “p value” can be regarded as more significant difference between objects to be compared.

Genes represented by “UP” are genes whose expression level is increased in Parkinson's disease patients, and genes represented by “DOWN” are genes whose expression level is decreased in Parkinson's disease patients.

The group of the differentially expressed genes described above was found to include genes related to Parkinson's disease (hsa05012) in search for a biological process (BP) and a KEGG pathway by gene ontology (GO) enrichment analysis (see Table 2 mentioned later). Meanwhile, in the group of the differentially expressed genes described above, genes shown in Tables 3-1 to 3-4 mentioned later are genes whose relation to Parkinson's disease has not been reported so far. Thus, at least one gene selected from the group consisting of these genes or an expression product thereof is a novel Parkinson's disease marker for detecting Parkinson's disease. Particularly, at least one gene selected from the group consisting of SNORA16A, SNORA24, SNORA50 and REXO1L2P which are common between Test 1 and Test 2, or an expression product thereof is preferred as a novel Parkinson's disease marker. Two or more genes selected from the group are more preferred, three or more genes selected therefrom are further more preferred, and all of the four genes are even more preferred. It is also preferred to include at least SNORA24, which is included in common in Table A described above and Table B mentioned later.

Differentially expressed RNA may be identified from data (read count values) on the expression level of RNA by using normalized count values obtained by using, for example, DESeq2 (Love M I et al., Genome Biol. 2014) or logarithmic values to base 2 of the count value plus integer 1 (Log₂(count+1) value).

For example, RNA which attains a corrected p value (FDR) of 0.25 or less in a likelihood ratio test in Parkinson's disease patients compared with healthy subjects is identified by using normalized count values as data on the expression level of RNA extracted from SSL of test subjects of the two tests mentioned above. As a result, 74 genes with increased expression, 209 genes with decreased expression, and a total of 283 genes (Tables 4-1 to 4-8) are obtained in Test 1, and 151 genes with increased expression, 308 genes with decreased expression, and a total of 459 genes (Tables 4-9 to 4-20) are obtained in Test 2. The expression of 7 genes is increased in common between Test 1 and Test 2 (ANXA1, AQP3, EMP1, KRT16, POLR2L, SERPINB4, and SNORA24), and the expression of 10 genes is decreased in common therebetween (ATP6VOC, BHLHE40, CCL3, CCNI, CXCR4, EGR2, GABARAPL1, RHOA, RNASEK, and SERINC1) (a total of 17 genes, Table B).

Thus, a gene selected from the group consisting of the 283 genes and the 459 genes (a total of 725 genes except for duplication) or an expression product thereof is capable of serving as a Parkinson's disease marker for detecting Parkinson's disease. Among them, a gene selected from the group consisting of the 17 genes shown in Table B or an expression product thereof is a preferred Parkinson's disease marker. Among them, a gene selected from the group consisting of 11 genes shown in Table C mentioned later, which are common with the genes shown in Table A described above, or an expression product thereof is a more preferred Parkinson's disease marker.

In the group of the differentially expressed genes described above, genes shown in Tables 6-1 and 6-2 mentioned later are genes whose relation to Parkinson's disease has not been reported so far. Thus, at least one gene selected from the group consisting of these genes or an expression product thereof is a novel Parkinson's disease marker for detecting Parkinson's disease. Particularly, SNORA24 (indicated by boldface in the table) which is common between Test 1 and Test 2 or an expression product thereof is preferred as a novel Parkinson's disease marker.

TABLE B

| | FDR | FDR | |
|---|---|---|---|
| Symbol | (Test 1) | (Test 2) | Regulation |
| ANXA1 | 0.032013 | 0.014395 | UP |
| AQP3 | 0.207454 | 0.197196 | UP |
| ATP6V0C | 0.142105 | 0.029799 | DOWN |
| BHLHE40 | 0.003239 | 0.189294 | DOWN |
| CCL3 | 0.022303 | 0.019217 | DOWN |
| CCNI | 8.89E−05 | 0.191526 | DOWN |
| CXCR4 | 0.024085 | 0.097541 | DOWN |
| EGR2 | 0.166431 | 0.179929 | DOWN |
| EMP1 | 0.060302 | 0.00062 | UP |
| GABARAPL1 | 0.060302 | 0.028215 | DOWN |
| KRT16 | 0.157035 | 0.203917 | UP |
| POLR2L | 0.205453 | 0.070687 | UP |
| RHOA | 0.166431 | 0.114613 | DOWN |
| RNASEK | 0.134092 | 0.189824 | DOWN |
| SERINC1 | 0.073126 | 0.233337 | DOWN |
| SERPINB4 | 0.093219 | 0.142882 | UP |
| SNORA24 | 0.022726 | 0.249405 | UP |

The gene capable of serving as a Parkinson's disease marker (hereinafter, also referred to as a “target gene”) also encompasses a gene having a nucleotide sequence substantially identical to the nucleotide sequence of DNA constituting the gene, as long as the gene is capable of serving as a biomarker for detecting Parkinson's disease. In this context, the nucleotide sequence substantially identical means a nucleotide sequence having 90% or higher, preferably 95% or higher, more preferably 98% or higher, further more preferably 99% or higher identity to the nucleotide sequence of DNA constituting the gene, for example, when searched by using homology calculation algorithm NCBI BLAST under conditions of expectation value=10; gap accepted; filtering=ON; match score=1; and mismatch score=−3.

The method for detecting Parkinson's disease according to the present invention includes a step of measuring an expression level of a target gene, which is in one aspect, at least one gene selected from the group consisting of SNORA16A, SNORA24, SNORA50 and REXO1L2P or an expression product thereof in a biological sample collected from a test subject.

In the method for detecting Parkinson's disease according to the present invention, examples of the test subject from which the biological sample is collected include mammals including humans and nonhuman mammals. A human is preferred. When the test subject is a human, the human is not particularly limited by sex, age, race, and the like thereof and can include infants to elderly people. Preferably, the test subject is a human who needs or desires detection of Parkinson's disease. The test subject is, for example, a human suspected of developing Parkinson's disease or a human having a genetic predisposition to develop Parkinson's disease.

The biological sample used in the present invention can be a tissue or a biomaterial in which the expression of the gene of the present invention varies with the onset or progression of Parkinson's disease. Examples thereof specifically include organs, skin, blood, urine, saliva, sweat, stratum corneum, skin surface lipids (SSL), body fluids such as tissue exudates, serum, plasma and others prepared from blood, feces, and hair, and preferably include the skin, the stratum corneum and skin surface lipids (SSL), more preferably skin surface lipids (SSL). Examples of the site of the skin from which SSL is collected include, but are not particularly limited to, the skin at an arbitrary site of the body, such as the head, the face, the neck, the body trunk, and the limbs. The skin at a site with high sebum secretion, for example, the skin of the head or the face, is preferred, and facial skin is more preferred.

In this context, the “skin surface lipids (SSL)” refer to a lipid-soluble fraction present on skin surface, and is also referred to as sebum. In general, SSL mainly contains secretion secreted from the exocrine gland such as the sebaceous gland in the skin, and is present on skin surface in the form of a thin layer that covers the skin surface. SSL contains RNA expressed in skin cells (see Patent Literature 3 described above). In the present specification, the “skin” is a generic name for regions containing tissues such as the stratum corneum, the epidermis, the dermis, and the hair follicle as well as the sweat gland, the sebaceous gland and other glands, unless otherwise specified.

Any approach for use in the recovery or removal of SSL from the skin can be adopted for the collection of SSL from the skin of a test subject. Preferably, an SSL-absorbent material or an SSL-adhesive material mentioned later, or a tool for scraping off SSL from the skin can be used. The SSL-absorbent material or the SSL-adhesive material is not particularly limited as long as the material has affinity for SSL. Examples thereof include polypropylene and pulp. More detailed examples of the procedure of collecting SSL from the skin include a method of allowing SSL to be absorbed to a sheet-like material such as an oil blotting paper or an oil blotting film, a method of allowing SSL to adhere to a glass plate, a tape, or the like, and a method of recovering SSL by scraping with a spatula, a scraper, or the like. In order to improve the adsorbability of SSL, an SSL-absorbent material impregnated in advance with a solvent having high lipid solubility may be used. On the other hand, the SSL-absorbent material preferably has a low content of a solvent having high water solubility or water because the adsorption of SSL to a material containing the solvent having high water solubility or water is inhibited. The SSL-absorbent material is preferably used in a dry state. Examples of the site of the skin from which SSL is collected include, but are not particularly limited to, the skin at an arbitrary site of the body, such as the head, the face, the neck, the body trunk, and the limbs. A site having high secretion of sebum, for example, the facial skin, is preferred.

The RNA-containing SSL collected from the test subject may be preserved for a given period. The collected SSL is preferably preserved under low-temperature conditions as rapidly as possible after collection in order to minimize the degradation of contained RNA. The temperature conditions for the preservation of RNA-containing SSL according to the present invention can be 0° C. or lower and are preferably from −20±20° C. to −80±20° C., more preferably from −20±10° C. to −80±10° C., further more preferably from −20±20° C. to −40±20° C., further more preferably from −20±10° C. to −40±10° C., further more preferably −20±10° C., further more preferably −20±5° C. The period of preservation of the RNA-containing SSL under the low-temperature conditions is not particularly limited and is preferably 12 months or shorter, for example, 6 hours or longer and 12 months or shorter, more preferably 6 months or shorter, for example, 1 day or longer and 6 months or shorter, further more preferably 3 months or shorter, for example, 3 days or longer and 3 months or shorter.

In the present invention, examples of the measurement object for the expression level of a target gene or an expression product thereof include cDNA artificially synthesized from RNA, DNA encoding the RNA, a protein encoded by the RNA, a molecule which interacts with the protein, a molecule which interacts with the RNA, and a molecule which interacts with the DNA. In this context, examples of the molecule which interacts with the RNA, the DNA or the protein include DNA, RNA, proteins, polysaccharides, oligosaccharides, monosaccharides, lipids, fatty acids, and their phosphorylation products, alkylation products, and sugar adducts, and complexes of any of them. The expression level comprehensively means the expression level or activity of the gene or the expression product.

In a preferred aspect, in the method of the present invention, SSL is used as a biological sample. In this case, the expression level of RNA contained in SSL is analyzed. Specifically, RNA is converted to cDNA through reverse transcription, followed by the measurement of the cDNA or an amplification product thereof.

In the extraction of RNA from SSL, a method which is usually used in RNA extraction or purification from a biological sample, for example, phenol/chloroform method, AGPC (acid guanidinium thiocyanate-phenol-chloroform extraction) method, a method using a column such as TRIzol®, RNeasy®, or QIAzol®, a method using special magnetic particles coated with silica, a method using magnetic particles for solid phase reversible immobilization, or extraction with a commercially available RNA extraction reagent such as ISOGEN can be used.

In the reverse transcription, primers which target particular RNA to be analyzed may be used, and random primers are preferably used for more comprehensive nucleic acid preservation and analysis. In the reverse transcription, common reverse transcriptase or reverse transcription reagent kit can be used. Highly accurate and efficient reverse transcriptase or reverse transcription reagent kit is suitably used. Examples thereof include M-MLV reverse transcriptase and its modified forms, and commercially available reverse transcriptase or reverse transcription reagent kits, for example, PrimeScript® Reverse Transcriptase series (Takara Bio Inc.) and SuperScript® Reverse Transcriptase series (Thermo Fisher Scientific, Inc.). SuperScript® III Reverse Transcriptase, SuperScript® VILO cDNA Synthesis kit (both from Thermo Fisher Scientific, Inc.), and the like are preferably used.

The temperature of extension reaction in the reverse transcription is adjusted to preferably 42° C.±1° C., more preferably 42° C.±0.5° C., further more preferably 42° C.±0.25° C., while its reaction time is adjusted to preferably 60 minutes or longer, more preferably from 80 to 120 minutes.

In the case of using RNA, cDNA or DNA as a measurement object, the method for measuring the expression level can be selected from nucleic acid amplification methods typified by PCR using DNA primers which hybridize thereto, real-time RT-PCR, multiplex PCR, SmartAmp, and LAMP, hybridization using a nucleic acid probe which hybridizes thereto (DNA chip, DNA microarray, dot blot hybridization, slot blot hybridization, Northern blot hybridization, and the like), a method of determining a nucleotide sequence (sequencing), and combined methods thereof.

In PCR, only particular DNA to be analyzed may be amplified by using a primer pair which targets the particular DNA, or a plurality of DNAs may be amplified by using a plurality of primer pairs. Preferably, the PCR is multiplex PCR. The multiplex PCR is a method of amplifying a plurality of gene regions at the same time by using a plurality of primer pairs at the same time in a PCR reaction system. The multiplex PCR can be carried out by using a commercially available kit (e.g., Ion AmpliSeq Transcriptome Human Gene Expression Kit; Life Technologies Japan Ltd.).

The temperature of annealing and extension reaction in the PCR depends on the primers used and therefore cannot be generalized. In the case of using the multiplex PCR kit described above, the temperature is preferably 62° C.±1° C., more preferably 62° C.±0.5° C., further more preferably 62° C.±0.25° C. Thus, preferably, the annealing and the extension reaction are performed by one step in the PCR. The time of the step of the annealing and the extension reaction can be adjusted depending on the size of DNA to be amplified, and the like, and is preferably from 14 to 18 minutes.

Conditions for denaturation reaction in the PCR can be adjusted depending on the DNA to be amplified, and are preferably from 95 to 99° C. and from 10 to 60 seconds. The reverse transcription and the PCR using the temperatures and the times as described above can be carried out by using a thermal cycler which is generally used for PCR.

The reaction product obtained by the PCR is preferably purified by the size separation of the reaction product. By the size separation, the PCR reaction product of interest can be separated from the primers and other impurities contained in the PCR reaction solution. The size separation of DNA can be performed by using, for example, a size separation column, a size separation chip, or magnetic beads which can be used in size separation. Preferred examples of the magnetic beads which can be used in size separation include magnetic beads for solid phase reversible immobilization (SPRI) such as Ampure XP.

The purified PCR reaction product may be subjected to further treatment necessary for conducting subsequent quantitative analysis. For example, for DNA sequencing, the purified PCR reaction product may be prepared into an appropriate buffer solution, the PCR primer regions contained in DNA amplified by PCR may be cleaved, and an adaptor sequence may be further added to the amplified DNA. For example, the purified PCR reaction product can be prepared into a buffer solution, and the removal of the PCR primer sequences and adaptor ligation can be performed for the amplified DNA. If necessary, the obtained reaction product can be amplified to prepare a library for quantitative analysis. These operations can be performed, for example, by using 5×VILO RT Reaction Mix attached to SuperScript® VILO cDNA Synthesis kit (Life Technologies Japan Ltd.), 5× Ion AmpliSeq HiFi Mix attached to Ion AmpliSeq Transcriptome Human Gene Expression Kit (Life Technologies Japan Ltd.), and Ion AmpliSeq Transcriptome Human Gene Expression Core Panel according to a protocol attached to each kit.

In the case of measuring the expression level of a target gene or a nucleic acid derived therefrom by use of Northern blot hybridization, examples thereof include a method in which; probe DNA is first labeled with a radioisotope, a fluorescent material, or the like. Subsequently, the obtained labeled DNA is allowed to hybridize to biological sample-derived RNA transferred to a nylon membrane or the like in accordance with a routine method. Then, the formed duplex of the labeled DNA and the RNA can be measured by detecting a signal derived from the label.

In the case of measuring the expression level of a target gene or a nucleic acid derived therefrom by use of RT-PCR, for example, cDNA is first prepared from biological sample-derived RNA in accordance with a routine method. This cDNA is used as a template, and a pair of primers (a positive strand which binds to the cDNA (− strand) and an opposite strand which binds to a + strand) prepared so as to be able to amplify the target gene of the present invention is allowed to hybridize thereto. Then, PCR is performed in accordance with a routine method, and the obtained amplified double-stranded DNA is detected. In the detection of the amplified double-stranded DNA, for example, a method of detecting labeled double-stranded DNA produced by the PCR by using primers labeled in advance with RI, a fluorescent material, or the like can be used.

In the case of measuring the expression level of a target gene or a nucleic acid derived therefrom by use of a DNA microarray, for example, an array in which at least one nucleic acid (cDNA or DNA) derived from the target gene of the present invention is immobilized on a support is used. Labeled cDNA or cRNA prepared from mRNA is allowed to bind onto the microarray, and the expression level of the mRNA can be measured by detecting the label on the microarray.

The nucleic acid to be immobilized in the array can be a nucleic acid which specifically (i.e., substantially only to the nucleic acid of interest) hybridizes under stringent conditions, and may be, for example, a nucleic acid having the whole sequence of the target gene of the present invention or may be a nucleic acid consisting of a partial sequence thereof. In this context, examples of the “partial sequence” include nucleic acids consisting of at least 15 to 25 bases. In this context, examples of the stringent conditions can usually include washing conditions on the order of “1×SSC, 0.1% SDS, and 37° C.”. Examples of the more stringent hybridization conditions can include conditions on the order of “0.5×SSC, 0.1% SDS, and 42° C.”. Examples of the much more stringent hybridization conditions can include conditions on the order of “0.1×SSC, 0.1% SDS, and 65° C.”. The hybridization conditions are described in, for example, J. Sambrook et al., Molecular Cloning: A Laboratory Manual, Third Edition, Cold Spring Harbor Laboratory Press (2001).

In the case of measuring the expression level of a target gene or a nucleic acid derived therefrom by sequencing, examples thereof include analysis using a next-generation sequencer (e.g., Ion S5/XL system, Life Technologies Japan Ltd.). RNA expression can be quantified on the basis of the number of reads (read count) prepared by the sequencing.

The probe or the primers for use in the measurement described above, which correspond to the primers for specifically recognizing and amplifying the target gene of the present invention or a nucleic acid derived therefrom, or the probe for specifically detecting the RNA or the nucleic acid derived therefrom, can be designed on the basis of a nucleotide sequence constituting the target gene. In this context, the phrase “specifically recognize” means that a detected product or an amplification product can be confirmed to be the gene or the nucleic acid derived therefrom in such a way that, for example, substantially only the target gene of the present invention or the nucleic acid derived therefrom can be detected in Northern blot, or, for example, substantially only the nucleic acid is amplified in RT-PCR.

Specifically, an oligonucleotide containing a given number of nucleotides complementary to DNA consisting of a nucleotide sequence constituting the target gene of the present invention, or a complementary strand thereof can be used. In this context, the “complementary strand” refers to one strand of double-stranded DNA consisting of A:T (U for RNA) and/or G:C base pairs with respect to the other strand. The term “complementary” is not limited by the case of being a completely complementary sequence in a region with the given number of consecutive nucleotides, and can have preferably 80% or higher, more preferably 90% or higher, further more preferably 95% or higher identity of the nucleotide sequence. The identity of the nucleotide sequence can be determined by algorithm such as BLAST described above.

For use as a primer, the oligonucleotide can achieve specific annealing and strand extension. Examples thereof usually include oligonucleotides having a strand length of 10 or more bases, preferably 15 or more bases, more preferably 20 or more bases, and 100 or less bases, preferably 50 or less bases, more preferably 35 or less bases. For use as a probe, the oligonucleotide can achieve specific hybridization. An oligonucleotide can be used which has at least a portion or the whole of the sequence of DNA (or a complementary strand thereof) consisting of a nucleotide sequence constituting the target gene of the present invention, and has a strand length of, for example, 10 or more bases, preferably 15 or more bases, and, for example, 100 or less bases, preferably 50 or less bases, more preferably 25 or less bases.

In this context, the “oligonucleotide” can be DNA or RNA and may be synthetic or natural. The probe for use in hybridization is usually labeled for use.

In the case of measuring a translation product (protein) of the target gene of the present invention, a molecule which interacts with the protein, a molecule which interacts with the RNA, or a molecule which interacts with the DNA, a method such as protein chip analysis, immunoassay (e.g., ELISA), mass spectrometry (e.g., LC-MS/MS and MALDI-TOF/MS), one-hybrid method (PNAS 100, 12271-12276 (2003)), or two-hybrid method (Biol. Reprod. 58, 302-311 (1998)) can be used and can be appropriately selected depending on the measurement object.

For example, in the case of using the protein as a measurement object, the measurement may be carried out by contacting an antibody against the expression product of the present invention with a biological sample, detecting a polypeptide in the sample bound to the antibody, and measuring the level thereof. For example, according to Western blot, the antibody described above is used as a primary antibody, and an antibody which binds to the primary antibody and which is labeled with, for example, a radioisotope, a fluorescent material or an enzyme is used as a secondary antibody to label the primary antibody therewith, followed by the measurement of a signal derived from such a labeling material using a radiation meter, a fluorescence detector, or the like.

The antibody against the translation product may be a polyclonal antibody or a monoclonal antibody. These antibodies can be produced in accordance with a method known in the art. Specifically, the polyclonal antibody may be produced by using a protein which has been expressed in E. coli or the like and purified in accordance with a routine method, or synthesizing a partial polypeptide of the protein in accordance with a routine method, and immunizing a nonhuman animal such as a house rabbit therewith, followed by obtainment from the serum of the immunized animal in accordance with a routine method.

Meanwhile, the monoclonal antibody can be obtained from hybridoma cells prepared by immunizing a nonhuman animal such as a mouse with a protein which has been expressed in E. coli or the like and purified in accordance with a routine method, or a partial polypeptide of the protein, and fusing the obtained spleen cells with myeloma cells. Alternatively, the monoclonal antibody may be prepared by use of phage display (Griffiths, A. D.; Duncan, A. R., Current Opinion in Biotechnology, Volume 9, Number 1, February 1998, pp. 102-108 (7)).

In this way, the expression level of the target gene of the present invention or the expression product thereof in a biological sample collected from a test subject is measured, and Parkinson's disease is detected on the basis of the expression level. The detection is specifically performed by comparing the measured expression level of the target gene of the present invention or the expression product thereof with a control level.

In the case of analyzing expression levels of a plurality of target genes by sequencing, as described above, read count values which are data on expression levels, RPM values which normalize the read count values for difference in the total number of reads among samples, values obtained by the conversion of the RPM values to logarithmic values to base 2 (Log₂ RPM values), or normalized count values obtained by using DESeq2 or logarithmic values to base 2 of the count value plus integer 1 (Log₂(count+1) values) are preferably used as an index. Also, values calculated by, for example, fragments per kilobase of exon per million reads mapped (FPKM), reads per kilobase of exon per million reads mapped (RPKM), or transcripts per million (TPM) which are general quantitative values of RNA-seq may be used. Alternatively, signal values obtained by microarray method or corrected values thereof may be used. In the case of analyzing only a particular target gene by RT-PCR or the like, an analysis method of converting the expression level of the target gene to a relative expression level with respect to the expression level of a housekeeping gene as a standard, or a method of analyzing a copy number obtained by absolute quantification using a plasmid containing a region of the target gene is preferred. A copy number obtained by digital PCR may be used.

In this context, examples of the “control level” include an expression level of the target gene or the expression product thereof in a healthy person. The expression level of the healthy person may be a statistic (e.g., a mean) of the expression level of the gene or the expression product thereof measured from a healthy person population. For a plurality of target genes, it is preferred to determine a standard expression level of each individual gene or expression product thereof.

The detection of Parkinson's disease according to the present invention may be performed through an increase and/or decrease in the expression level of the target gene of the present invention or the expression product thereof. In this case, the expression level of the target gene or the expression product thereof in a biological sample derived from a test subject is compared with a cutoff value (reference value) of each gene or the expression product thereof. The cutoff value can be appropriately determined on the basis of a statistical numeric value, such as a mean or standard deviation, of the expression level based on the expression level of the target gene or expression product thereof in a healthy subject obtained as a standard data.

A discriminant (prediction model) which discriminates between a Parkinson's disease patient and a healthy person is constructed by using measurement values of an expression level of the target gene or the expression product thereof derived from a Parkinson's disease patient and an expression level of the target gene or the expression product thereof derived from a healthy person, and Parkinson's disease can be detected through the use of the discriminant. Specifically, a discriminant (prediction model) which discriminates between a Parkinson's disease patient and a healthy person is constructed by using measurement values of an expression level of a target gene or an expression product thereof derived from a Parkinson's disease patient and an expression level of the target gene or the expression product thereof derived from a healthy subject as teacher samples, and a cutoff value (reference value) which discriminates between the Parkinson's disease patient and the healthy person is determined on the basis of the discriminant. In the preparation of the discriminant, dimensional compression is performed by principal component analysis (PCA), and a principal component can be used as an explanatory variable.

The presence or absence of Parkinson's disease in a test subject can be evaluated by similarly measuring a level of the target gene or the expression product thereof from a biological sample collected from the test subject, substituting the obtained measurement value into the discriminant, and comparing the results obtained from the discriminant with the reference value.

In this context, algorithm known in the art such as algorithm for use in machine learning can be used as the algorithm in the construction of the discriminant. Examples of the machine learning algorithm include random forest, linear kernel support vector machine (SVM linear), rbf kernel support vector machine (SVM rbf), neural network, generalized linear model, regularized linear discriminant analysis, and regularized logistic regression. A predictive value is calculated by inputting data for the verification of the constructed prediction model, and a model which attains the predictive value most compatible with an actually measured value, for example, recall, precision, and an F value which is a harmonic mean thereof are calculated from a predictive value and an actually measured value, and a model having the largest F value can be selected as the optimum prediction model.

The method for determining the cutoff value (reference value) is not particularly limited, and the value can be determined in accordance with an approach known in the art. The value can be determined from, for example, an ROC (receiver operating characteristic) curve prepared by using the discriminant. In the ROC curve, the probability (%) of producing positive results in positive patients (sensitivity) is plotted on the ordinate against a value (false positive rate) of 1 minus the probability (%) of producing negative results in negative patients (specificity) on the abscissa. As for “true positive (sensitivity)” and “false positive (1−specificity)” shown in the ROC curve, a value at which “true positive (sensitivity)”−“false positive (1−specificity)” is maximized (Youden index) can be used as the cutoff value (reference value).

As shown in Examples mentioned later, prediction models were constructed by use of machine learning algorithm by using a value of each principal component obtained from expression level data (Log₂ RPM values) on target genes shown in Table A (33 genes or 4 genes selected therefrom) as an explanatory variable, and the healthy subjects and the Parkinson's disease patients as objective variables. As a result, Parkinson's disease was found predictable with the model by using the 4 genes SNORA16A, SNORA24, SNORA50, and REXO1L2P. Also, Parkinson's disease was found predictable more accurately with the model by using the 33 genes.

Thus, in the case of preparing the discriminant which discriminates between a Parkinson's disease patient group and a healthy person group, a discriminant which exhibits high recall and precision can be prepared by appropriately adding, to expression data on the 4 target genes SNORA16A, SNORA24, SNORA50 and REXO1L2P, expression data on at least one gene selected from the group consisting of the remaining 29 genes shown in Table A or an expression product thereof as a target gene, preferably adding thereto an appropriate number of genes with high variable importance based on variable importance shown in Table 8 mentioned later. Thus, Parkinson's disease can be detected with higher accuracy. Specifically, addition of 8 genes EGR2, RHOA, CCNI, RNASEK, CSF2RB, SERP1, ANKRD12, and SLC25A3 are preferred. Further, addition of 12 genes consisting of these 8 genes and 4 genes CD83, CXCR4, ITGAX, and UQCRH are preferred, and addition of 18 genes consisting of these 12 genes and 6 genes KCNQ1OT1, CCL3, C10orf116, SERPINB4, LCE3D, and CNFN are preferred. It is preferred to add all of the 29 genes.

Alternatively, expression data on at least one gene, except for SNORA24, selected from the group consisting of 11 genes which are shown as differentially expressed genes in both Table A and Table B described above, and shown in Table C given below, or an expression product thereof may be appropriately added as a target gene to the 4 target genes SNORA16A, SNORA24, SNORA50 and REXO1L2P.

TABLE C

| Symbol | Regulation |
|---|---|
| CCL3 | DOWN |
| CCNI | DOWN |
| CXCR4 | DOWN |
| EGR2 | DOWN |
| EMP1 | UP |
| POLR2L | UP |
| RHOA | DOWN |
| RNASEK | DOWN |
| SERINC1 | DOWN |
| SERPINB4 | UP |
| SNORA24 | UP |

Expression data on at least one gene selected from the group consisting of genes shown in Table B or an expression product thereof may be used as a target gene for use in preparing the discriminant which discriminates between a Parkinson's disease patient group and a healthy person group. Preferably, SNORA24 as well as at least one of the other genes is used. More preferably, expression data on genes shown in Table C or expression products thereof is used. Further more preferably, expression data on all the genes shown in Table B or expression products thereof is used.

The test kit for detecting Parkinson's disease according to the present invention contains a test reagent for measuring an expression level of the target gene of the present invention or an expression product thereof in a biological sample separated from a patient.

Specific examples thereof include a reagent for nucleic acid amplification and hybridization containing an oligonucleotide (e.g., a primer for PCR) which specifically binds (hybridizes) to the target gene of the present invention or a nucleic acid derived therefrom, and a reagent for immunoassay containing an antibody which recognizes an expression product (protein) of the target gene of the present invention. The oligonucleotide, the antibody, or the like contained in the kit can be obtained by a method known in the art as mentioned above.

The test kit may contain, in addition to the antibody or the nucleic acid, a labeling reagent, a buffer solution, a chromogenic substrate, a secondary antibody, a blocking agent, an instrument necessary for a test, a control, a tool for collecting a biological sample (e.g., an oil blotting film for collecting SSL), and the like.

Aspects and preferred embodiments of the present invention will be given below.

<1> A method for detecting Parkinson's disease in a test subject, comprising a step of measuring an expression level of at least one gene selected from the group of 4 genes consisting of SNORA16A, SNORA24, SNORA50 and REXO1L2P or an expression product thereof in a biological sample collected from the test subject.

<2> The method for detecting Parkinson's disease according to <1>, wherein the method at least comprises measuring an expression level of SNORA24 gene or an expression product thereof.

<3> The method according to <1> or <2>, wherein the expression level of the gene or the expression product thereof is measured as an expression level of mRNA.

<4> The method according to any of <1> to <3>, wherein the gene or the expression product thereof is RNA contained in skin surface lipids of the test subject.

<5> The method according to any of <1> to <4>, wherein the presence or absence of Parkinson's disease is evaluated by comparing the measurement value of the expression level with a reference value of the gene or the expression product thereof.

<6> The method according to any of <1> to <4>, wherein the presence or absence of Parkinson's disease in the test subject is evaluated by the following steps: preparing a discriminant which discriminates between the Parkinson's disease patient and a healthy person by using measurement values of an expression level of the gene or the expression product thereof derived from a Parkinson's disease patient and an expression level of the gene or the expression product thereof derived from a healthy subject as teacher samples; substituting the measurement value of the expression level of the gene or the expression product thereof obtained from the biological sample collected from the test subject into the discriminant; and comparing the obtained results with a reference value.

<7> The method according to <6>, wherein expression levels of all the genes of the group of 4 genes or expression products thereof are measured.

<8> The method according to <6> or <7>, wherein expression levels of the at least one gene selected from the group of 4 genes as well as at least one gene selected from the following group of 29 genes or expression products thereof are measured:

ANKRD12, C10orf116, CCL3, CCNI, CD83, CNFN, CNN2, CSF2RB, CXCR4, EGR2, EMP1, ITGAX, KCNQ1OT1, LCE3D, LITAF, NDUFA4L2, NDUFS5, POLR2L, RHOA, RNASEK, RPL7A, RPS26, SERINC1, SERP1, SERPINB4, SLC25A3, SNRPG, SRRM2, and UQCRH.

<9> The method according to <8>, wherein expression levels of the at least one gene selected from the group of 4 genes as well as at least one gene selected from the following group of 10 genes or expression products thereof are measured:

CCL3, CCNI, CXCR4, EGR2, EMP1, POLR2L, RHOA, RNASEK, SERINC1, and SERPINB4.

<10> The method according to <6> or <7>, wherein expression levels of the at least one gene selected from the group of 4 genes as well as at least one gene selected from the following group of 16 genes or expression products thereof are measured:

ANXA1, AQP3, ATP6VOC, BHLHE40, CCL3, CCNI, CXCR4, EGR2, EMP1, GABARAPL1, KRT16, POLR2L, RHOA, RNASEK, SERINC1, and SERPINB4.

<11> The method according to <6> or <7>, wherein expression levels of the at least one gene selected from the group of 4 genes as well as at least one gene selected from the groups of genes shown in Tables 3-1 to 3-4 mentioned later and Tables 6-1 and 6-2 mentioned later (except for the 4 genes) or expression products thereof are measured.

<12> The method according to <6> or <7>, wherein expression levels of the at least one gene selected from the group of 4 genes as well as at least one gene selected from the groups of 1,005 genes shown in Tables 1-1 to 1-27 mentioned later and 725 genes shown in Tables 4-1 to 4-20 mentioned later except for the 4 genes or expression products thereof are measured.

<13> A test kit for detecting Parkinson's disease, the kit being used in a method according to any of <1> to <10>, and comprising an oligonucleotide which specifically hybridizes to the gene or a nucleic acid derived therefrom, or an antibody which recognizes an expression product of the gene.

<14> Use of at least one gene selected from the groups of genes shown in Tables 3-1 to 3-4 mentioned later and Tables 6-1 and 6-2 mentioned later or an expression product thereof as a marker for detecting Parkinson's disease.

<15> Use of at least one gene selected from the group of 4 genes consisting of SNORA16A, SNORA24, SNORA50 and REXO1L2P or an expression product thereof as a marker for detecting Parkinson's disease.

<16> A marker for detecting Parkinson's disease comprising at least one gene selected from the groups of genes shown in Tables 3-1 to 3-4 mentioned later and Tables 6-1 and 6-2 mentioned later or an expression product thereof.

<17> The marker for detecting Parkinson's disease according to <16>, wherein the detection marker comprises at least one gene selected from the group of 4 genes consisting of SNORA16A, SNORA24, SNORA50 and REXO1L2P or an expression product thereof.

### EXAMPLES

Hereinafter, the present invention will be described in more detail with reference to Examples. However, the present invention is not limited by these examples.

### Example 1 Detection of Parkinson's Disease by Using RNA Extracted from SSL

1) SSL Collection

Two tests were conducted as the following Test 1 and Test 2.

Test 1: 15 healthy subjects (from 40 to 89 years old, male and female) and 15 Parkinson's disease patients (PD) (from 40 to 89 years old, male and female) were selected as test subjects.

Test 2: 50 healthy subjects (from 40 to 89 years old, male) and 50 PD (from 40 to 89 years old, male) were selected as test subjects.

PD was diagnosed in advance as Parkinson's disease (Hoehn & Yahr stage I or II) by a neurologist. Sebum was recovered from the whole face of each test subject by using an oil blotting film (5×8 cm, made of polypropylene, 3M Company). Then, the oil blotting film was transferred to a vial and preserved at −80° C. for approximately 1 month until use in RNA extraction.

2) RNA Preparation and Sequencing

The oil blotting film of the above section 1) was cut into an appropriate size, and RNA was extracted by using QIAzol Lysis Reagent (Qiagen N.V.) in accordance with the attached protocol. On the basis of the extracted RNA, cDNA was synthesized through reverse transcription at 42° C. for 90 minutes by using SuperScript VILO cDNA Synthesis kit (Life Technologies Japan Ltd.). The primers used for reverse transcription reaction were random primers attached to the kit. A library containing DNA derived from 20802 genes was prepared by multiplex PCR from the obtained cDNA. The multiplex PCR was performed by using Ion AmpliSeq Transcriptome Human Gene Expression Kit (Life Technologies Japan Ltd.) under conditions of [99° C., 2 min→(99° C., 15 sec→62° C., 16 min)×20 cycles→4° C., hold]. The obtained PCR product was purified with Ampure XP (Beckman Coulter Inc.), followed by buffer reconstitution, primer sequence digestion, adaptor ligation, purification, and amplification to prepare a library. The prepared library was loaded on Ion 540 Chip and sequenced by using Ion S5/XL system (Life Technologies Japan Ltd.).

3) Data Analysis

i) RNA Expression Analysis—1

In the data (read count values) on the expression level of RNA derived from the test subjects measured in the above section 2), data with a read count of less than 10 was treated as missing values. After conversion to RPM values which normalized the read count values for difference in the total number of reads among samples, the missing values were compensated for by use of an approach called singular value decomposition (SVD) imputation. However, only genes which produced expression level data without missing values in 80% or more sample test subjects in the expression level data on the test subjects in all the samples were used in analysis given below. In the analysis, converted RPM values, logarithmic values of the RPM values of the read counts to base 2 (Log₂ RPM values) were used in order to approximate the RPM values, which followed negative binominal distribution, to normal distribution.

Differentially expressed RNA which attained a p value of 0.05 or less in Student's t-test in PD compared with the healthy subjects was identified on the basis of the SSL-derived RNA expression levels (Log₂ RPM values) of the healthy subjects and PD described above. In Test 1, the expression of 111 RNAs was increased in PD compared with the healthy subjects (Tables 1-1 to 1-3), and the expression of 68 RNAs was decreased therein (Tables 1-4 to 1-5). Meanwhile, in Test 2, the expression of 565 RNAs was increased (Tables 1-6 to 1-19), and the expression of 294 RNAs was decreased (Tables 1-20 to 1-27). The expression of 18 RNAs was increased in common between Test 1 and Test 2, and the expression of 15 RNAs was decreased in common therebetween (genes indicated by boldface in the tables).

TABLE 1-1

| Test | Symbol | Fold change | p value | Regulation |
|---|---|---|---|---|
| Test 1 | ADRM1 | 0.609210571 | 0.043833254 | UP |
| Test 1 | ARF5 | 0.699874992 | 0.026663956 | UP |
| Test 1 | ARHGEF5 | 1.274057568 | 0.048234993 | UP |
| Test 1 | BCKDK | 1.080907515 | 0.000291562 | UP |
| Test 1 | C10orf116 | 1.346318684 | 0.027914601 | UP |
| Test 1 | C11orf10 | 1.167882659 | 9.62E−05 | UP |
| Test 1 | C14orf2 | 0.627373658 | 0.013138616 | UP |
| Test 1 | CEBPA | 1.625049985 | 0.001261191 | UP |
| Test 1 | CHAC1 | 1.615056048 | 0.017192687 | UP |
| Test 1 | CHCHD2 | 0.867580331 | 0.009229477 | UP |
| Test 1 | CMIP | 0.555282243 | 0.042367806 | UP |
| Test 1 | CNFN | 1.272119089 | 0.024347366 | UP |
| Test 1 | COPE | 0.889122418 | 0.006852965 | UP |
| Test 1 | COPS8 | 1.11272686 | 0.033587705 | UP |
| Test 1 | COX8A | 0.614359496 | 0.037782725 | UP |
| Test 1 | CSDA | 1.34167409 | 0.00133884 | UP |
| Test 1 | CTBP2 | 0.962196486 | 0.024818568 | UP |
| Test 1 | CTDNEP1 | 0.961690787 | 0.019367259 | UP |
| Test 1 | CYFIP1 | 1.075131825 | 0.044852257 | UP |
| Test 1 | DADI | 0.860983925 | 0.020003683 | UP |
| Test 1 | DNASE1L2 | 1.461274581 | 0.021696734 | UP |
| Test 1 | DUX4L4 | 1.821703773 | 0.016937626 | UP |
| Test 1 | EDF1 | 0.634696647 | 0.006062601 | UP |
| Test 1 | EIF3E | 0.85054249 | 0.023427635 | UP |
| Test 1 | EIF4G1 | 0.874582442 | 0.008300118 | UP |
| Test 1 | EMP1 | 1.428292097 | 0.010451584 | UP |
| Test 1 | FAM129B | 0.906162295 | 0.03314269 | UP |
| Test 1 | FAM83G | 1.692703352 | 0.005470943 | UP |
| Test 1 | FEM1B | 1.475450334 | 0.00508158 | UP |
| Test 1 | G6PD | 0.767453922 | 0.046885133 | UP |
| Test 1 | GPBP1L1 | 0.943160553 | 0.030787333 | UP |
| Test 1 | GPR157 | 1.399710796 | 0.001639686 | UP |
| Test 1 | GPX3 | 1.106976877 | 0.011928668 | UP |
| Test 1 | HECA | 0.551678309 | 0.021073296 | UP |
| Test 1 | HIPK1 | 1.10213078 | 0.008842706 | UP |
| Test 1 | HIST2H2BE | 0.639444381 | 0.048606096 | UP |
| Test 1 | HLA.DQB2 | 1.515233848 | 0.034824195 | UP |
| Test 1 | HMGCS1 | 0.852395823 | 0.033976968 | UP |
| Test 1 | HSPA1A | 1.559694806 | 0.002025704 | UP |
| Test 1 | IQSEC1 | 1.33654119 | 0.01105081 | UP |
| Test 1 | KCNQ1OT1 | 1.644199329 | 0.04140012 | UP |

TABLE 1-2

| | | | | |
|---|---|---|---|---|
| Test 1 | KCTD11 | 1.172625927 | 0.028996311 | UP |
| Test 1 | KIAA0930 | 0.802565628 | 0.040645349 | UP |
| Test 1 | KLHDC3 | 1.162575929 | 0.029124485 | UP |
| Test 1 | LCE3D | 1.525057902 | 0.017729736 | UP |
| Test 1 | LOC100093631 | 1.202317044 | 0.018702745 | UP |
| Test 1 | LOC100506888 | 2.345734653 | 0.005026134 | UP |
| Test 1 | LOC349196 | 2.155586353 | 0.025593893 | UP |
| Test 1 | LOC401321 | 1.818317716 | 0.043316136 | UP |
| Test 1 | LPIN1 | 1.342620463 | 0.022057267 | UP |
| Test 1 | MAP2K2 | 0.96303756 | 0.018140774 | UP |
| Test 1 | METRNL | 0.495872438 | 0.034081797 | UP |
| Test 1 | MGLL | 1.12786717 | 0.031415315 | UP |
| Test 1 | NDUFA13 | 1.028288472 | 0.002609224 | UP |
| Test 1 | NDUFA4L2 | 1.469853745 | 0.047011103 | UP |
| Test 1 | NDUFB11 | 1.178953409 | 0.015560192 | UP |
| Test 1 | NDUFS5 | 1.173366098 | 0.028285636 | UP |
| Test 1 | NR4A3 | 1.062128975 | 0.018538 | UP |
| Test 1 | OAZ1 | 0.46509906 | 0.019553364 | UP |
| Test 1 | OR4F3 | 1.980298573 | 0.013883164 | UP |
| Test 1 | PKP3 | 1.051268637 | 0.017270523 | UP |
| Test 1 | POLD4 | 0.727728607 | 0.026390396 | UP |
| Test 1 | POLR2L | 1.288069119 | 0.005102443 | UP |
| Test 1 | PPA1 | 0.749279023 | 0.03206103 | UP |
| Test 1 | PQLC1 | 0.790392011 | 0.038455602 | UP |
| Test 1 | PRELID1 | 0.895128216 | 0.036353629 | UP |
| Test 1 | PSMB7 | 1.198078264 | 0.010069291 | UP |
| Test 1 | PSMC1 | 0.921786669 | 0.002813666 | UP |
| Test 1 | PSMD4 | 1.162613293 | 0.000301746 | UP |
| Test 1 | PURB | 1.294350134 | 0.005492975 | UP |
| Test 1 | RAP2B | 0.712661882 | 0.007752025 | UP |
| Test 1 | RASALI | 1.533954936 | 0.007931264 | UP |
| Test 1 | REXO1L2P | 2.258334633 | 0.021096388 | UP |
| Test 1 | RPL7A | 0.799765552 | 0.040024088 | UP |
| Test 1 | RPS26 | 1.048925589 | 0.020173699 | UP |
| Test 1 | RRAD | 1.551857659 | 0.009621785 | UP |
| Test 1 | RRAGA | 1.21815067 | 0.01109446 | UP |
| Test 1 | SEC61A1 | 1.061101156 | 0.045353369 | UP |
| Test 1 | SERPINB4 | 1.73450959 | 0.048165225 | UP |
| Test 1 | SFXN3 | 0.967423668 | 0.026897044 | UP |

TABLE 1-3

| | | | | |
|---|---|---|---|---|
| Test 1 | SLC25A3 | 0.683663369 | 0.040816858 | UP |
| Test 1 | SNF8 | 0.92398189 | 0.031993765 | UP |
| Test 1 | SNORA16A | 1.233214856 | 0.005217419 | UP |
| Test 1 | SNORA24 | 1.397191537 | 0.001016782 | UP |
| Test 1 | SNORA43 | 1.274218723 | 0.007468774 | UP |
| Test 1 | SNORA50 | 1.299388426 | 0.010607324 | UP |
| Test 1 | SNORA8 | 1.005454688 | 0.028833348 | UP |
| Test 1 | SNRPG | 1.989577925 | 0.002505629 | UP |
| Test 1 | SPINT1 | 1.207565409 | 0.034516318 | UP |
| Test 1 | SQRDL | 0.766969993 | 0.00457474 | UP |
| Test 1 | SRXN1 | 0.873833451 | 0.018173048 | UP |
| Test 1 | STAT6 | 1.011794726 | 0.007483321 | UP |
| Test 1 | STIP1 | 0.85103506 | 0.043705392 | UP |
| Test 1 | TALDO1 | 0.536260527 | 0.045700866 | UP |
| Test 1 | TCEB3CL | 2.366983401 | 0.006731453 | UP |
| Test 1 | TCIRG1 | 1.57092833 | 0.027056141 | UP |
| Test 1 | TEX264 | 1.236719119 | 0.014308363 | UP |
| Test 1 | TMEM183A | 1.156156624 | 0.03501219 | UP |
| Test 1 | TRMT112 | 0.867005398 | 0.033782415 | UP |
| Test 1 | TTC9 | 0.883705752 | 0.014677092 | UP |
| Test 1 | TYMP | 1.201700537 | 0.0171455 | UP |
| Test 1 | UQCRB | 0.761955105 | 0.036349839 | UP |
| Test 1 | UQCRC1 | 1.021819204 | 0.000400463 | UP |
| Test 1 | UQCRH | 1.081064513 | 0.010579673 | UP |
| Test 1 | UQCRQ | 1.172007584 | 0.009792679 | UP |
| Test 1 | USP17L5 | 2.261956901 | 0.017602717 | UP |
| Test 1 | USP17L6P | 2.333811727 | 0.013336494 | UP |
| Test 1 | USP38 | 1.129519222 | 0.022350671 | UP |
| Test 1 | VEGFA | 0.958877042 | 0.010007452 | UP |
| Test 1 | ZNF33A | 1.458704981 | 0.009711608 | UP |
| Test 1 | ZNF410 | 0.936467529 | 0.017554463 | UP |

TABLE 1-4

| | | | | |
|---|---|---|---|---|
| Test 1 | ACSL1 | −1.314414944 | 0.021386968 | DOWN |
| Test 1 | ACSL4 | −1.040837462 | 0.017095652 | DOWN |
| Test 1 | ANKRD12 | −1.930754522 | 0.010768568 | DOWN |
| Test 1 | ARPC1B | −0.617322479 | 0.042888509 | DOWN |
| Test 1 | BRD4 | −1.024694066 | 0.02763023 | DOWN |
| Test 1 | BTG1 | −0.934321414 | 0.039327439 | DOWN |
| Test 1 | CALM2 | −0.91810275 | 0.009187675 | DOWN |
| Test 1 | CCL3 | −1.639111096 | 0.008678309 | DOWN |
| Test 1 | CCNI | −1.932387295 | 0.00403203 | DOWN |
| Test 1 | CD83 | −1.066374053 | 0.04175246 | DOWN |
| Test 1 | CDC42 | −1.611732634 | 0.00284614 | DOWN |
| Test 1 | CHMP4B | −0.715746914 | 0.030901 | DOWN |
| Test 1 | CNBP | −1.045580294 | 0.00387558 | DOWN |
| Test 1 | CNN2 | −0.629754604 | 0.023710615 | DOWN |
| Test 1 | CSF2RB | −1.104312619 | 0.020573046 | DOWN |
| Test 1 | CXCR4 | −2.033830014 | 0.00024412 | DOWN |
| Test 1 | DDX5 | −1.149683056 | 0.023786744 | DOWN |
| Test 1 | EEF1A1 | −0.734882964 | 0.008509423 | DOWN |
| Test 1 | EEE1B2 | −0.972404288 | 0.009011592 | DOWN |
| Test 1 | EGR2 | −0.997120306 | 0.005989411 | DOWN |
| Test 1 | EIF1 | −1.068314684 | 0.000455452 | DOWN |
| Test 1 | EPS15 | −1.047381411 | 0.006922926 | DOWN |
| Test 1 | GNG10 | −0.739434103 | 0.04672473 | DOWN |
| Test 1 | GRINA | −0.709755929 | 0.043785466 | DOWN |
| Test 1 | H3F3A | −0.484685703 | 0.042106784 | DOWN |
| Test 1 | HIF1A | −0.738592709 | 0.038912439 | DOWN |
| Test 1 | HNRNPA2B1 | −1.27064282 | 0.001794524 | DOWN |
| Test 1 | HNRNPU | −1.02784524 | 0.042720575 | DOWN |
| Test 1 | IFNGR2 | −1.000333459 | 0.013306532 | DOWN |
| Test 1 | ILIRN | −1.291864782 | 0.0026776 | DOWN |
| Test 1 | ITGAX | −1.11377676 | 0.027930711 | DOWN |
| Test 1 | LITAF | −0.831805644 | 0.014085655 | DOWN |
| Test 1 | LYN | −0.959021868 | 0.040941384 | DOWN |
| Test 1 | NEAT1 | −0.957011121 | 0.047894721 | DOWN |
| Test 1 | PABPC1 | −1.035504388 | 0.002341174 | DOWN |
| Test 1 | PAIP2 | −0.864545414 | 0.040428904 | DOWN |
| Test 1 | PGK1 | −0.963519428 | 0.011841674 | DOWN |
| Test 1 | PLXNC1 | −1.099466822 | 0.026428919 | DOWN |
| Test 1 | RABGEF1 | −0.993856958 | 0.037254884 | DOWN |
| Test 1 | RAP1A | −1.114744033 | 0.031169618 | DOWN |
| Test 1 | REL | −1.282793738 | 0.01153212 | DOWN |

TABLE 1-5

| | | | | |
|---|---|---|---|---|
| Test 1 | RGS2 | −0.995907178 | 0.045303026 | DOWN |
| Test 1 | RHOA | −0.902566363 | 0.003151667 | DOWN |
| Test 1 | RNASEK | −1.016194703 | 0.030620951 | DOWN |
| Test 1 | RPL10 | −2.025185976 | 5.92E−05 | DOWN |
| Test 1 | RPL15 | −1.54290515 | 0.000469071 | DOWN |
| Test 1 | RPL19 | −0.892202011 | 0.015862788 | DOWN |
| Test 1 | RPL21 | −0.625280627 | 0.036709641 | DOWN |
| Test 1 | RPL26 | −1.153562245 | 0.015851768 | DOWN |
| Test 1 | RPL28 | −1.000169058 | 0.030081325 | DOWN |
| Test 1 | RPL3 | −1.077830298 | 0.003267945 | DOWN |
| Test 1 | RPL30 | −0.660396387 | 0.033024638 | DOWN |
| Test 1 | RPL35 | −0.700317983 | 0.029053156 | DOWN |
| Test 1 | RPL5 | −1.081758489 | 0.0300877 | DOWN |
| Test 1 | RPL6 | −1.573868621 | 0.025108045 | DOWN |
| Test 1 | RPS20 | −1.311993027 | 0.023283488 | DOWN |
| Test 1 | RPS25 | −0.913868434 | 0.022273799 | DOWN |
| Test 1 | S100A11 | −1.226240532 | 0.001687759 | DOWN |
| Test 1 | SCARNA9 | −1.045209104 | 0.029905065 | DOWN |
| Test 1 | SERINC1 | −0.651103256 | 0.046063301 | DOWN |
| Test 1 | SERP1 | −0.82729507 | 0.033858187 | DOWN |
| Test 1 | SNORA53 | −1.226150595 | 0.046365671 | DOWN |
| Test 1 | SRRM2 | −0.752261071 | 0.036848008 | DOWN |
| Test 1 | STK24 | −1.185703307 | 0.03897646 | DOWN |
| Test 1 | TMEM127 | −0.800780218 | 0.025357034 | DOWN |
| Test 1 | TNIP1 | −1.01072003 | 0.008782635 | DOWN |
| Test 1 | TPM4 | −0.629827116 | 0.033794869 | DOWN |
| Test 1 | TPT1 | −0.672287453 | 0.035475508 | DOWN |

TABLE 1-6

| | | | | |
|---|---|---|---|---|
| Test 2 | A2ML1 | 1.221533613 | 0.000112481 | UP |
| Test 2 | ABRACL | 0.595825415 | 0.002340761 | UP |
| Test 2 | ACBD3 | 0.480880204 | 0.019490426 | UP |
| Test 2 | ACOT13 | 0.435902885 | 0.027959053 | UP |
| Test 2 | ACSS3 | 0.639119766 | 0.023601116 | UP |
| Test 2 | ADAP2 | 0.538749812 | 0.018924703 | UP |
| Test 2 | ADPRHL2 | 0.404739489 | 0.045086548 | UP |
| Test 2 | ADSL | 0.4588524 | 0.038972385 | UP |
| Test 2 | ADSS | 0.651140057 | 0.002197834 | UP |
| Test 2 | AHCY | 0.802441979 | 0.000607218 | UP |
| Test 2 | AIF1L | 1.055021255 | 0.000495159 | UP |
| Test 2 | AIM1L | 0.62944081 | 0.040629376 | UP |
| Test 2 | AKI | 0.491622387 | 0.029441855 | UP |
| Test 2 | AK4 | 0.474128267 | 0.041622944 | UP |
| Test 2 | ALDH1A3 | 0.535322936 | 0.025214627 | UP |
| Test 2 | ALDOC | 0.471800562 | 0.013096482 | UP |
| Test 2 | AMBRA1 | 0.359110063 | 0.04974087 | UP |
| Test 2 | ANP32B | 0.397177548 | 0.03932888 | UP |
| Test 2 | ANP32E | 0.494174712 | 0.011258347 | UP |
| Test 2 | ANXA1 | 0.54435181 | 0.008220099 | UP |
| Test 2 | AP4S1 | 0.553991355 | 0.012146838 | UP |
| Test 2 | ARFGAP2 | 0.459611753 | 0.018201769 | UP |
| Test 2 | ARHGAP29 | 0.914208609 | 0.003531249 | UP |
| Test 2 | ARL1 | 0.408729114 | 0.044051381 | UP |
| Test 2 | ASS1 | 0.643120811 | 0.006574816 | UP |
| Test 2 | ATP5B | 0.249687247 | 0.039711327 | UP |
| Test 2 | ATP5E | 0.284815562 | 0.033935216 | UP |
| Test 2 | ATP5G1 | 0.599572218 | 0.003312854 | UP |
| Test 2 | ATP5I | 0.537663746 | 0.00267032 | UP |
| Test 2 | ATP5O | 0.39709147 | 0.002208756 | UP |
| Test 2 | ATPIF1 | 0.382294733 | 0.01863468 | UP |
| Test 2 | BAG3 | 0.716644595 | 0.005145384 | UP |
| Test 2 | BCAS1 | 0.948535572 | 0.005105851 | UP |
| Test 2 | BCAS2 | 0.440711713 | 0.003048003 | UP |
| Test 2 | BCL2L13 | 0.458803119 | 0.037434143 | UP |
| Test 2 | BCL7C | 0.443147949 | 0.039568486 | UP |
| Test 2 | BMP2 | 0.68056004 | 0.032195249 | UP |
| Test 2 | C10orf116 | 0.529752336 | 0.039587014 | UP |
| Test 2 | C11orf31 | 0.358444195 | 0.020591887 | UP |
| Test 2 | C1orf52 | 0.395191044 | 0.049654449 | UP |
| Test 2 | C1orf63 | 0.45668822 | 0.026824533 | UP |

TABLE 1-7

| | | | | |
|---|---|---|---|---|
| Test 2 | C22orf32 | 0.509669614 | 0.03126071 | UP |
| Test 2 | C2orf49 | 0.601493547 | 0.000740502 | UP |
| Test 2 | C5orf43 | 0.343779718 | 0.040727567 | UP |
| Test 2 | C5orf46 | 0.745587247 | 0.009981759 | UP |
| Test 2 | C8orf33 | 0.451060206 | 0.025475738 | UP |
| Test 2 | CACYBP | 0.426591512 | 0.016609089 | UP |
| Test 2 | CALM1 | 0.330924338 | 0.004677453 | UP |
| Test 2 | CARHSP1 | 0.784681465 | 0.000175932 | UP |
| Test 2 | CASK | 0.599582959 | 0.01003174 | UP |
| Test 2 | CASP14 | 0.632903218 | 0.027505259 | UP |
| Test 2 | CAST | 0.350973694 | 0.030849173 | UP |
| Test 2 | CCDC6 | 0.696273484 | 0.003551549 | UP |
| Test 2 | CCNE1 | 0.555426503 | 0.037173127 | UP |
| Test 2 | CCT2 | 0.427873529 | 0.031204993 | UP |
| Test 2 | CCT3 | 0.352019271 | 0.042821173 | UP |
| Test 2 | CCT4 | 0.414581271 | 0.048774852 | UP |
| Test 2 | CCT8 | 0.3837055 | 0.049670658 | UP |
| Test 2 | CDC16 | 0.57034355 | 0.009643665 | UP |
| Test 2 | CDSN | 0.644348354 | 0.010053349 | UP |
| Test 2 | CGA | 1.091914746 | 0.000462077 | UP |
| Test 2 | CGNL1 | 0.9992731 | 0.000612501 | UP |
| Test 2 | CHI3L2 | 0.57424439 | 0.020046259 | UP |
| Test 2 | CHIC2 | 0.410002803 | 0.04762423 | UP |
| Test 2 | CHMP4A | 0.52527036 | 0.004336838 | UP |
| Test 2 | CIZ1 | 0.498982985 | 0.039336744 | UP |
| Test 2 | CKB | 0.683086969 | 0.018305412 | UP |
| Test 2 | CLIC3 | 0.74263737 | 0.020412012 | UP |
| Test 2 | CLIP1 | 0.435971364 | 0.019001211 | UP |
| Test 2 | CNDP2 | 0.281021946 | 0.048982715 | UP |
| Test 2 | CNFN | 0.990121666 | 1.85E−05 | UP |
| Test 2 | CNIH4 | 0.457328621 | 0.02164633 | UP |
| Test 2 | CNN3 | 0.624509347 | 0.016593952 | UP |
| Test 2 | CNNM4 | 0.561756946 | 0.049794296 | UP |
| Test 2 | COA1 | 0.635059866 | 0.00138502 | UP |
| Test 2 | COA3 | 0.55836372 | 0.010330091 | UP |
| Test 2 | COMT | 0.329211212 | 0.046136915 | UP |
| Test 2 | COX4I1 | 0.298413672 | 0.00394488 | UP |
| Test 2 | COX5B | 0.236438786 | 0.036474931 | UP |
| Test 2 | CPEB2 | 0.875753539 | 0.003788596 | UP |
| Test 2 | CPNE3 | 0.532838867 | 0.015475427 | UP |
| Test 2 | CRABP2 | 0.766900027 | 0.000735812 | UP |

TABLE 1-8

| | | | | |
|---|---|---|---|---|
| Test 2 | CRELD2 | 0.685045483 | 0.009010899 | UP |
| Test 2 | CRIPT | 0.623088661 | 0.000802774 | UP |
| Test 2 | CRNN | 1.401884112 | 0.001214875 | UP |
| Test 2 | CST6 | 0.589966531 | 0.016466862 | UP |
| Test 2 | CSTA | 0.784255116 | 0.002502729 | UP |
| Test 2 | CUL4A | 0.489558405 | 0.013487958 | UP |
| Test 2 | CUTA | 0.585987127 | 0.001016471 | UP |
| Test 2 | CYB5A | 0.641939407 | 0.009668198 | UP |
| Test 2 | CYB5B | 0.544680118 | 0.005232354 | UP |
| Test 2 | DANCR | 0.43126336 | 0.041709971 | UP |
| Test 2 | DCAF12 | 0.545454648 | 0.011615314 | UP |
| Test 2 | DDRGK1 | 0.372347748 | 0.044309125 | UP |
| Test 2 | DDT | 0.472760004 | 0.010925051 | UP |
| Test 2 | DEGS1 | 0.545984689 | 0.037107489 | UP |
| Test 2 | DENND2C | 0.502288792 | 0.047224257 | UP |
| Test 2 | DHPS | 0.518845683 | 0.012866877 | UP |
| Test 2 | DHX29 | 0.682106105 | 0.006066935 | UP |
| Test 2 | DHX32 | 0.506509259 | 0.033864568 | UP |
| Test 2 | DHX40 | 0.396573604 | 0.015540946 | UP |
| Test 2 | DNAJA1 | 0.252552372 | 0.019713754 | UP |
| Test 2 | DNAJA4 | 0.483045351 | 0.044641278 | UP |
| Test 2 | DNAJC13 | 0.470362936 | 0.029093404 | UP |
| Test 2 | DNAJC15 | 0.469211563 | 0.013979287 | UP |
| Test 2 | DNAJC21 | 0.452709072 | 0.022814459 | UP |
| Test 2 | DNAJC7 | 0.319676387 | 0.022220543 | UP |
| Test 2 | DNAJC9 | 0.575126954 | 0.012161694 | UP |
| Test 2 | DOCK6 | 0.545719783 | 0.03676478 | UP |
| Test 2 | DOCK9 | 0.621757986 | 0.012261459 | UP |
| Test 2 | DPH1 | 1.158039818 | 6.72E−05 | UP |
| Test 2 | DPY30 | 0.398317757 | 0.02828779 | UP |
| Test 2 | DRG1 | 0.581253641 | 0.004984247 | UP |
| Test 2 | DSG1 | 0.567399218 | 0.037732972 | UP |
| Test 2 | DUSP11 | 0.473325618 | 0.006136292 | UP |
| Test 2 | DYM | 0.816178513 | 0.00274631 | UP |
| Test 2 | DYNC1LI1 | 0.583242858 | 0.0067388 | UP |
| Test 2 | DYNLL1 | 0.374636406 | 0.035010075 | UP |
| Test 2 | DYNLRB1 | 0.330053674 | 0.027194242 | UP |
| Test 2 | ECHSI | 0.374219263 | 0.043974114 | UP |
| Test 2 | EFNB2 | 0.661019693 | 0.019887237 | UP |
| Test 2 | EIF1AX | 0.600523864 | 0.00135969 | UP |
| Test 2 | EIF2S2 | 0.666962534 | 0.008564954 | UP |

TABLE 1-9

| | | | | |
|---|---|---|---|---|
| Test 2 | EIF3K | 0.47571023 | 0.000332146 | UP |
| Test 2 | EIF4EBP1 | 0.509037765 | 0.036582232 | UP |
| Test 2 | ELOVL7 | 0.663055469 | 0.029361928 | UP |
| Test 2 | EMP1 | 0.948607145 | 0.000952753 | UP |
| Test 2 | ENDOD1 | 0.83064568 | 0.003155686 | UP |
| Test 2 | EPHB6 | 0.996531897 | 0.000172709 | UP |
| Test 2 | EPHX3 | 0.957706717 | 0.001357233 | UP |
| Test 2 | ERBB3 | 0.668574797 | 0.018620686 | UP |
| Test 2 | ERO1L | 0.595297117 | 0.005178102 | UP |
| Test 2 | EXOC4 | 0.717282266 | 0.003430647 | UP |
| Test 2 | EXOC5 | 0.555371778 | 0.003548179 | UP |
| Test 2 | EXOC6B | 0.472278499 | 0.047161361 | UP |
| Test 2 | F13A1 | 0.700312885 | 0.033387875 | UP |
| Test 2 | FABP4 | 1.470499552 | 2.97E−05 | UP |
| Test 2 | FABP9 | 1.368332459 | 2.70E−05 | UP |
| Test 2 | FAM108B1 | 0.629521772 | 0.011810697 | UP |
| Test 2 | FAM135A | 0.588280986 | 0.032230354 | UP |
| Test 2 | FAM210B | 0.545271557 | 0.037981153 | UP |
| Test 2 | FAM25B | 0.522599819 | 0.047412373 | UP |
| Test 2 | FAM3C | 0.615760366 | 0.004593036 | UP |
| Test 2 | FAM45A | 0.552544411 | 0.016999174 | UP |
| Test 2 | FAM46B | 0.791235807 | 0.003269448 | UP |
| Test 2 | FBXO45 | 0.651269976 | 0.015000916 | UP |
| Test 2 | FCHSD1 | 0.505831668 | 0.048614621 | UP |
| Test 2 | FIG4 | 0.441222277 | 0.010793265 | UP |
| Test 2 | FKBP1A | 0.163295342 | 0.048754238 | UP |
| Test 2 | FKBP3 | 0.635315923 | 0.011710114 | UP |
| Test 2 | FLG | 0.8220595 | 0.030652941 | UP |
| Test 2 | FOXQ1 | 0.739538694 | 0.019924301 | UP |
| Test 2 | FRMD6 | 0.680800391 | 0.007307527 | UP |
| Test 2 | FTSJ1 | 0.66863706 | 0.005309815 | UP |
| Test 2 | FUNDC2 | 0.499543676 | 0.016301607 | UP |
| Test 2 | FYN | 0.464677144 | 0.036203854 | UP |
| Test 2 | GBAS | 0.702529543 | 0.002848186 | UP |
| Test 2 | GGCT | 0.642905928 | 0.026758206 | UP |
| Test 2 | GHITM | 0.25443743 | 0.032592325 | UP |
| Test 2 | GLOD4 | 0.533853822 | 0.013415796 | UP |
| Test 2 | GNL3 | 0.584900087 | 0.009442293 | UP |
| Test 2 | GPSM2 | 0.739454628 | 0.002548872 | UP |
| Test 2 | GRHL3 | 0.572937195 | 0.024076507 | UP |
| Test 2 | GRPEL1 | 0.471178948 | 0.007047655 | UP |

TABLE 1-10

| | | | | |
|---|---|---|---|---|
| Test 2 | GTF2A2 | 0.330372647 | 0.040423351 | UP |
| Test 2 | GTF2E2 | 0.534855078 | 0.004830931 | UP |
| Test 2 | GTF2H5 | 0.611879208 | 0.000741758 | UP |
| Test 2 | GTF3C5 | 0.388985663 | 0.032287492 | UP |
| Test 2 | GTF3C6 | 0.562851294 | 0.008550842 | UP |
| Test 2 | H1FX | 0.38289824 | 0.039887459 | UP |
| Test 2 | HADH | 0.596886384 | 0.031698277 | UP |
| Test 2 | HBEGF | 0.35824757 | 0.029353225 | UP |
| Test 2 | HDAC1 | 0.412996826 | 0.019794177 | UP |
| Test 2 | HDDC2 | 0.481028865 | 0.038549638 | UP |
| Test 2 | HEATR5A | 0.541675769 | 0.004141004 | UP |
| Test 2 | HEXB | 0.48326638 | 0.016112958 | UP |
| Test 2 | HIBADH | 0.491409911 | 0.028149152 | UP |
| Test 2 | HIBCH | 0.588943801 | 0.025448145 | UP |
| Test 2 | HIST1H1E | 0.436334476 | 0.040849694 | UP |
| Test 2 | HIST1H2AE | 0.472022185 | 0.032486571 | UP |
| Test 2 | HIST1H2AG | 0.554952196 | 0.026912916 | UP |
| Test 2 | HIST1H2AI | 0.53748617 | 0.034833553 | UP |
| Test 2 | HIST1H2AM | 0.505465922 | 0.015356542 | UP |
| Test 2 | HIST1H2BN | 0.547150364 | 0.019828836 | UP |
| Test 2 | HIST1H3B | 1.061476948 | 0.000400823 | UP |
| Test 2 | HIST1H3I | 0.601309393 | 0.011107396 | UP |
| Test 2 | HIST1H4B | 0.839544468 | 0.00079634 | UP |
| Test 2 | HIST1H4E | 0.778335085 | 0.00020329 | UP |
| Test 2 | HIST1H4F | 0.551175791 | 0.032237462 | UP |
| Test 2 | HIST1H4H | 0.715081702 | 0.000190121 | UP |
| Test 2 | HMOX2 | 0.375124592 | 0.032277484 | UP |
| Test 2 | HNRNPA0 | 0.43012224 | 0.023849018 | UP |
| Test 2 | HOMER1 | 0.572056122 | 0.027336542 | UP |
| Test 2 | HOOK1 | 0.689647412 | 0.000609804 | UP |
| Test 2 | HPGD | 0.531461662 | 0.034525209 | UP |
| Test 2 | HRSP12 | 0.748163886 | 0.00429738 | UP |
| Test 2 | HSD17B10 | 0.525580431 | 0.005390788 | UP |
| Test 2 | HSP90AA1 | 0.450671514 | 0.012853222 | UP |
| Test 2 | HSPD1 | 0.353524268 | 0.038337878 | UP |
| Test 2 | HYPK | 0.495508732 | 0.000812946 | UP |
| Test 2 | IDE | 0.561486404 | 0.030266606 | UP |
| Test 2 | IDH3A | 0.715741483 | 0.000740982 | UP |
| Test 2 | IFI27 | 1.088718271 | 0.000166105 | UP |
| Test 2 | IL32 | 0.635464648 | 0.022927371 | UP |
| Test 2 | IL36A | 1.193557169 | 0.000147742 | UP |

TABLE 1-11

| | | | | |
|---|---|---|---|---|
| Test 2 | ILKAP | 0.498704265 | 0.018877961 | UP |
| Test 2 | IPO5 | 0.524135485 | 0.004351655 | UP |
| Test 2 | IQCG | 0.517533726 | 0.033045998 | UP |
| Test 2 | ITGB1BP1 | 0.592037248 | 0.005471131 | UP |
| Test 2 | ITPA | 0.428949095 | 0.023930788 | UP |
| Test 2 | ITPRIPL2 | 0.522695359 | 0.014016549 | UP |
| Test 2 | IVL | 1.113959428 | 4.72E−05 | UP |
| Test 2 | KANK1 | 0.722652018 | 0.016004319 | UP |
| Test 2 | KCNQ1OT1 | 0.517120259 | 0.015543571 | UP |
| Test 2 | KIAA0240 | 0.431683501 | 0.018876409 | UP |
| Test 2 | KIAA1143 | 0.346950172 | 0.046415853 | UP |
| Test 2 | KLF5 | 0.500232931 | 0.027794858 | UP |
| Test 2 | KLK13 | 0.65799937 | 0.017034932 | UP |
| Test 2 | KLK7 | 0.744388154 | 0.007376759 | UP |
| Test 2 | KLK8 | 0.597855011 | 0.024365979 | UP |
| Test 2 | KRT14 | 0.471660604 | 0.041821956 | UP |
| Test 2 | KRT16 | 0.438210052 | 0.041610329 | UP |
| Test 2 | KRT25 | 1.299645558 | 5.17E−05 | UP |
| Test 2 | KRT26 | 0.707262572 | 0.008809601 | UP |
| Test 2 | KRT27 | 1.207014606 | 5.40E−05 | UP |
| Test 2 | KRT5 | 0.757037273 | 0.027824563 | UP |
| Test 2 | KRT6A | 0.454023277 | 0.038422784 | UP |
| Test 2 | KRT6C | 0.78340478 | 0.023768169 | UP |
| Test 2 | KRT71 | 1.159098826 | 8.74E−05 | UP |
| Test 2 | KRT72 | 1.230166904 | 6.27E−05 | UP |
| Test 2 | KRT74 | 1.095061209 | 0.00590623 | UP |
| Test 2 | KRT78 | 0.737035195 | 0.034619014 | UP |
| Test 2 | KRTAP1.5 | 1.60464646 | 0.000819038 | UP |
| Test 2 | KRTAP12.1 | 1.229397362 | 0.000287135 | UP |
| Test 2 | KRTAP12.2 | 0.938688052 | 0.001073077 | UP |
| Test 2 | KRTAP19.1 | 0.844471097 | 0.018428031 | UP |
| Test 2 | KRTAP3.1 | 2.122465083 | 7.02E−05 | UP |
| Test 2 | KRTAP3.3 | 1.394541092 | 0.001179985 | UP |
| Test 2 | KRTAP5.3 | 1.432908956 | 6.99E−05 | UP |
| Test 2 | KRTAP5.7 | 1.447966369 | 3.78E−05 | UP |
| Test 2 | KRTDAP | 0.702409287 | 0.003662818 | UP |
| Test 2 | KTN1 | 0.381285498 | 0.039123884 | UP |
| Test 2 | LCE2A | 0.582131055 | 0.026199523 | UP |
| Test 2 | LCE2C | 0.619920884 | 0.015817916 | UP |
| Test 2 | LCE2D | 0.621960127 | 0.021384422 | UP |
| Test 2 | LCE3D | 0.843482517 | 0.000577787 | UP |

TABLE 1-12

| | | | | |
|---|---|---|---|---|
| Test 2 | LCE3E | 0.836563972 | 0.000810209 | UP |
| Test 2 | LCMT1 | 0.601842869 | 0.025859542 | UP |
| Test 2 | LCN2 | 0.73325772 | 0.003321 | UP |
| Test 2 | LEMD3 | 0.440994015 | 0.016865308 | UP |
| Test 2 | LEPROTL1 | 0.377879515 | 0.038317178 | UP |
| Test 2 | LINC00675 | 0.573523306 | 0.034073972 | UP |
| Test 2 | LLPH | 0.484998299 | 0.007188702 | UP |
| Test 2 | LMBR1 | 0.665083353 | 0.00191151 | UP |
| Test 2 | LNX1 | 0.952713443 | 0.000293549 | UP |
| Test 2 | LOC100505738 | 0.477053745 | 0.024409 | UP |
| Test 2 | LOC550643 | 0.634672437 | 0.002533558 | UP |
| Test 2 | LOC646862 | 0.747572165 | 0.025405318 | UP |
| Test 2 | LRBA | 0.529280351 | 0.038783597 | UP |
| Test 2 | LRRC15 | 0.906456672 | 0.002321669 | UP |
| Test 2 | LSM10 | 0.508507242 | 0.013416263 | UP |
| Test 2 | LSM2 | 0.675878285 | 0.004242908 | UP |
| Test 2 | LSM7 | 0.570617764 | 0.005193872 | UP |
| Test 2 | LTF | 0.717042662 | 0.012011178 | UP |
| Test 2 | LY6D | 0.638909247 | 0.038220601 | UP |
| Test 2 | LYNX1 | 1.006012262 | 0.002091327 | UP |
| Test 2 | MAFA | 0.646509839 | 0.018661569 | UP |
| Test 2 | MAL | 1.157393695 | 0.00203966 | UP |
| Test 2 | MALL | 1.082853546 | 0.000258882 | UP |
| Test 2 | MAOA | 0.489452289 | 0.017793881 | UP |
| Test 2 | MAP4K3 | 0.567499535 | 0.022681249 | UP |
| Test 2 | MAP7 | 0.597783019 | 0.034525239 | UP |
| Test 2 | MCCC1 | 0.677783016 | 0.008565533 | UP |
| Test 2 | MCTS1 | 0.499448675 | 0.013734219 | UP |
| Test 2 | MICALCL | 0.519128213 | 0.00748038 | UP |
| Test 2 | MNF1 | 0.448890025 | 0.045325106 | UP |
| Test 2 | MPHOSPH6 | 0.431463962 | 0.044290704 | UP |
| Test 2 | MPV17 | 0.462010792 | 0.022209637 | UP |
| Test 2 | MRPL11 | 0.444169953 | 0.041198724 | UP |
| Test 2 | MRPL12 | 0.459260738 | 0.023664309 | UP |
| Test 2 | MRPL24 | 0.49423042 | 0.034251269 | UP |
| Test 2 | MRPL32 | 0.570527545 | 0.004685957 | UP |
| Test 2 | MRPL47 | 0.522250156 | 0.004855696 | UP |
| Test 2 | MRPS11 | 0.723572233 | 0.000171238 | UP |
| Test 2 | MRPS18B | 0.606642285 | 0.003402311 | UP |
| Test 2 | MRPS24 | 0.424610103 | 0.027109976 | UP |
| Test 2 | MT1X | 0.913147816 | 0.000517578 | UP |

TABLE 1-13

| | | | | |
|---|---|---|---|---|
| Test 2 | MTMR12 | 0.60532463 | 0.015109514 | UP |
| Test 2 | MUT | 0.529761761 | 0.005829175 | UP |
| Test 2 | MYO10 | 0.937118688 | 6.65E−05 | UP |
| Test 2 | MZT2A | 0.830391158 | 0.000900666 | UP |
| Test 2 | NCBP2 | 0.513307919 | 0.007048167 | UP |
| Test 2 | NCK1 | 0.49374477 | 0.015371978 | UP |
| Test 2 | NDRG2 | 0.354843551 | 0.048575543 | UP |
| Test 2 | NDUFA12 | 0.737096619 | 0.000119869 | UP |
| Test 2 | NDUFA2 | 0.432617464 | 0.013454569 | UP |
| Test 2 | NDUFA4L2 | 1.063393782 | 2.72E−05 | UP |
| Test 2 | NDUFB1 | 0.287495056 | 0.035078556 | UP |
| Test 2 | NDUFS5 | 0.457090069 | 0.011340643 | UP |
| Test 2 | NDUFS6 | 0.590046428 | 6.11E−05 | UP |
| Test 2 | NEDD4L | 0.511884957 | 0.049453884 | UP |
| Test 2 | NFU1 | 0.65767548 | 0.002449743 | UP |
| Test 2 | NHP2 | 0.454032668 | 0.040871766 | UP |
| Test 2 | NIN | 0.52183109 | 0.010001048 | UP |
| Test 2 | NIPAL3 | 0.577629247 | 0.046987766 | UP |
| Test 2 | NIPAL4 | 0.897027632 | 0.005310037 | UP |
| Test 2 | NOSIP | 0.427938626 | 0.020360295 | UP |
| Test 2 | NRIP3 | 0.506284789 | 0.020884863 | UP |
| Test 2 | NSMCE1 | 0.577791701 | 0.00730131 | UP |
| Test 2 | NUDC | 0.694341809 | 0.014762563 | UP |
| Test 2 | NUMA1 | 0.36191125 | 0.020606246 | UP |
| Test 2 | NUP214 | 0.47743027 | 0.049271574 | UP |
| Test 2 | NUPL1 | 0.335741644 | 0.047223087 | UP |
| Test 2 | OFD1 | 0.563849491 | 0.004983374 | UP |
| Test 2 | OLA1 | 0.393966653 | 0.029910839 | UP |
| Test 2 | ORMDL3 | 0.439255773 | 0.034802361 | UP |
| Test 2 | PABPN1 | 0.350869528 | 0.02211862 | UP |
| Test 2 | PADI1 | 0.67070604 | 0.019434864 | UP |
| Test 2 | PADI3 | 1.215749009 | 7.35E−05 | UP |
| Test 2 | PAK4 | 0.509965072 | 0.042007684 | UP |
| Test 2 | PAPL | 0.529409941 | 0.037223187 | UP |
| Test 2 | PCCB | 0.709869935 | 0.001272684 | UP |
| Test 2 | PDCD5 | 0.377965927 | 0.044465853 | UP |
| Test 2 | PDDC1 | 0.503218985 | 0.028228175 | UP |
| Test 2 | PDE12 | 0.396547298 | 0.041406947 | UP |
| Test 2 | PDHA1 | 0.492613289 | 0.011811977 | UP |
| Test 2 | PDZD8 | 0.455694983 | 0.039833582 | UP |
| Test 2 | PDZK1IP1 | 0.748234961 | 0.004749239 | UP |

TABLE 1-14

| | | | | |
|---|---|---|---|---|
| Test 2 | PEPD | 0.468814718 | 0.026975661 | UP |
| Test 2 | PFDN2 | 0.4710158 | 0.021859005 | UP |
| Test 2 | PFDN5 | 0.220335663 | 0.049147318 | UP |
| Test 2 | PFDN6 | 0.494260171 | 0.011519195 | UP |
| Test 2 | PHAX | 0.508298395 | 0.018098917 | UP |
| Test 2 | PHF13 | 0.460509877 | 0.034666845 | UP |
| Test 2 | PHPT1 | 0.548135369 | 0.005002792 | UP |
| Test 2 | PICK1 | 0.4635477 | 0.02617828 | UP |
| Test 2 | PINLYP | 0.783929639 | 0.006215794 | UP |
| Test 2 | PITRM1 | 0.39895903 | 0.039173248 | UP |
| Test 2 | PKP1 | 0.644729487 | 0.011720844 | UP |
| Test 2 | PLCD1 | 0.737870521 | 0.005536531 | UP |
| Test 2 | PLD2 | 0.508558382 | 0.012373652 | UP |
| Test 2 | PLS3 | 0.658472726 | 0.007445336 | UP |
| Test 2 | POF1B | 0.848689586 | 0.009264505 | UP |
| Test 2 | POLR2D | 0.4992488 | 0.001983556 | UP |
| Test 2 | POLR2G | 0.598027214 | 0.010404429 | UP |
| Test 2 | POLR2L | 0.3357497 | 0.037600455 | UP |
| Test 2 | POLR2M | 0.411455592 | 0.045454349 | UP |
| Test 2 | PPFIBP2 | 0.449798477 | 0.042506262 | UP |
| Test 2 | PPID | 0.382456948 | 0.041407629 | UP |
| Test 2 | PPIL4 | 0.40788823 | 0.035051455 | UP |
| Test 2 | PPL | 1.165630089 | 0.000661664 | UP |
| Test 2 | PPP1R13B | 0.707903942 | 0.012009575 | UP |
| Test 2 | PPP2R2A | 0.37767129 | 0.046357843 | UP |
| Test 2 | PPP5C | 0.814821426 | 0.002376737 | UP |
| Test 2 | PPWD1 | 0.580223957 | 0.004372047 | UP |
| Test 2 | PRDX3 | 0.409111767 | 0.024794403 | UP |
| Test 2 | PRDX6 | 0.310519039 | 0.032277997 | UP |
| Test 2 | PREP | 0.416055344 | 0.048166689 | UP |
| Test 2 | PRKRA | 0.45076589 | 0.028570612 | UP |
| Test 2 | PROM2 | 0.821663343 | 0.016405122 | UP |
| Test 2 | PRPF40A | 0.457875591 | 0.027986636 | UP |
| Test 2 | PRPF4B | 0.537856846 | 0.002990068 | UP |
| Test 2 | PRR9 | 1.012665648 | 0.000286425 | UP |
| Test 2 | PRSS3 | 0.753484738 | 0.001895958 | UP |
| Test 2 | PSMC2 | 0.35148635 | 0.040165519 | UP |
| Test 2 | PSORS1C2 | 0.946759355 | 0.006100019 | UP |
| Test 2 | PTPN3 | 0.543446291 | 0.035596652 | UP |
| Test 2 | PVRL4 | 0.80686893 | 0.004930709 | UP |
| Test 2 | QKI | 0.277755845 | 0.031937093 | UP |

TABLE 1-15

| | | | | |
|---|---|---|---|---|
| Test 2 | RAB38 | 0.748487957 | 0.001035725 | UP |
| Test 2 | RABIF | 0.438505415 | 0.007298243 | UP |
| Test 2 | RANBP1 | 1.04696268 | 1.36E−05 | UP |
| Test 2 | RANBP10 | 0.552977796 | 0.026238115 | UP |
| Test 2 | RARRES1 | 0.620687381 | 0.019855473 | UP |
| Test 2 | RBM10 | 0.425725495 | 0.023147642 | UP |
| Test 2 | RBMS2 | 0.706576019 | 0.001389874 | UP |
| Test 2 | REXO1L2P | 0.730041651 | 0.016022131 | UP |
| Test 2 | RHCG | 1.163040682 | 9.35E−05 | UP |
| Test 2 | RMRP | 0.605574492 | 0.000163029 | UP |
| Test 2 | RNASE7 | 0.718897169 | 0.019766232 | UP |
| Test 2 | RNF121 | 0.440046558 | 0.016046961 | UP |
| Test 2 | RNF20 | 0.673608418 | 0.00223791 | UP |
| Test 2 | ROMO1 | 0.325128448 | 0.038508067 | UP |
| Test 2 | RPA1 | 0.436121484 | 0.045123305 | UP |
| Test 2 | RPIA | 0.74921433 | 0.000111086 | UP |
| Test 2 | RPL10A | 0.283649048 | 0.039968527 | UP |
| Test 2 | RPL18 | 0.301532043 | 0.024095949 | UP |
| Test 2 | RPL21 | 0.266798928 | 0.03678764 | UP |
| Test 2 | RPL26L1 | 0.674511114 | 0.001010617 | UP |
| Test 2 | RPL30 | 0.231637085 | 0.039116984 | UP |
| Test 2 | RPL32 | 0.357709343 | 0.004112163 | UP |
| Test 2 | RPL36 | 0.311893187 | 0.018234435 | UP |
| Test 2 | RPL36A | 0.336279436 | 0.007725217 | UP |
| Test 2 | RPL37A | 0.415682354 | 0.003350758 | UP |
| Test 2 | RPL38 | 0.285259201 | 0.027892997 | UP |
| Test 2 | RPL7 | 0.355361637 | 0.006023648 | UP |
| Test 2 | RPL7A | 0.370261967 | 0.003107308 | UP |
| Test 2 | RPLP0 | 0.397845736 | 0.002167296 | UP |
| Test 2 | RPLP1 | 0.422223519 | 0.001209864 | UP |
| Test 2 | RPS12 | 0.45913525 | 0.000751779 | UP |
| Test 2 | RPS15 | 0.302865045 | 0.029280518 | UP |
| Test 2 | RPS15A | 0.260829012 | 0.042894741 | UP |
| Test 2 | RPS18 | 0.549381525 | 0.00019412 | UP |
| Test 2 | RPS26 | 0.423684057 | 0.015281796 | UP |
| Test 2 | RPS28 | 0.376721184 | 0.01216883 | UP |
| Test 2 | RPS29 | 0.98999607 | 0.001861427 | UP |
| Test 2 | RPS3 | 0.438411389 | 0.005266268 | UP |
| Test 2 | RPS4X | 0.381013466 | 0.004861978 | UP |
| Test 2 | RPS5 | 0.351386701 | 0.014125176 | UP |
| Test 2 | RPS6 | 0.278963462 | 0.044007702 | UP |

TABLE 1-16

| | | | | |
|---|---|---|---|---|
| Test 2 | RPS6KA2 | 0.517206191 | 0.04372271 | UP |
| Test 2 | RPS6KB1 | 0.413890467 | 0.04890732 | UP |
| Test 2 | RPTN | 0.792988732 | 0.039812579 | UP |
| Test 2 | S100A14 | 0.735444547 | 0.007361184 | UP |
| Test 2 | S100A7 | 0.511938094 | 0.039234587 | UP |
| Test 2 | S100A7A | 0.780884027 | 0.006392561 | UP |
| Test 2 | S100A8 | 0.480554943 | 0.005565746 | UP |
| Test 2 | S100A9 | 0.541890412 | 0.001827719 | UP |
| Test 2 | SBDS | 0.501382901 | 0.004032371 | UP |
| Test 2 | SBF1 | 0.450478584 | 0.026502631 | UP |
| Test 2 | SBSN | 0.514431135 | 0.03450677 | UP |
| Test 2 | SCARNA12 | 0.47613506 | 0.013100706 | UP |
| Test 2 | SCARNA16 | 0.950996515 | 4.89E−06 | UP |
| Test 2 | SCARNA17 | 0.480107492 | 0.005539693 | UP |
| Test 2 | SCARNA6 | 0.69159554 | 0.000174868 | UP |
| Test 2 | SCARNA7 | 0.39276757 | 0.018164776 | UP |
| Test 2 | SCGB2A2 | 0.605410508 | 0.04509638 | UP |
| Test 2 | SCNNIB | 0.790814471 | 0.006130053 | UP |
| Test 2 | SCNNIG | 0.659327124 | 0.033550311 | UP |
| Test 2 | SDR16C5 | 0.803745452 | 0.005092806 | UP |
| Test 2 | SDR9C7 | 0.562729748 | 0.023941492 | UP |
| Test 2 | SEC23A | 0.381494906 | 0.048213143 | UP |
| Test 2 | SERPINA9 | 0.745391039 | 0.009476658 | UP |
| Test 2 | SERPINB4 | 0.740104652 | 0.009405167 | UP |
| Test 2 | SERPINB5 | 0.545369808 | 0.01702846 | UP |
| Test 2 | SERPINB7 | 0.996392198 | 0.001276768 | UP |
| Test 2 | SF3B14 | 0.322347433 | 0.03198073 | UP |
| Test 2 | SF3B3 | 0.374175675 | 0.042316392 | UP |
| Test 2 | SH3GL3 | 0.536171647 | 0.014877267 | UP |
| Test 2 | SLC10A6 | 0.70193016 | 0.012397218 | UP |
| Test 2 | SLC25A20 | 0.506425898 | 0.031631881 | UP |
| Test 2 | SLC25A3 | 0.266515461 | 0.031602198 | UP |
| Test 2 | SLC25A5 | 0.348945458 | 0.046829004 | UP |
| Test 2 | SLC26A9 | 0.875604042 | 0.003342367 | UP |
| Test 2 | SLC5A1 | 0.69571361 | 0.019710856 | UP |
| Test 2 | SLC6A14 | 1.057914573 | 0.000130014 | UP |
| Test 2 | SLC6A8 | 0.649208632 | 0.00790547 | UP |
| Test 2 | SLFN5 | 0.484567716 | 0.031313993 | UP |
| Test 2 | SLMO2 | 0.422924252 | 0.044372872 | UP |
| Test 2 | SLURPI | 1.144320913 | 0.003648741 | UP |
| Test 2 | SMAD7 | 0.460254587 | 0.028410104 | UP |

TABLE 1-17

| | | | | |
|---|---|---|---|---|
| Test 2 | SMC3 | 0.424518141 | 0.025409105 | UP |
| Test 2 | SMEK2 | 0.365851287 | 0.025427854 | UP |
| Test 2 | SMIM5 | 0.578361256 | 0.035681779 | UP |
| Test 2 | SNHG1 | 0.574921517 | 0.043671047 | UP |
| Test 2 | SNHG16 | 0.452555179 | 0.016900832 | UP |
| Test 2 | SNHG6 | 0.544935945 | 0.015263998 | UP |
| Test 2 | SNHG9 | 0.482721161 | 0.016394879 | UP |
| Test 2 | SNIP1 | 0.530290658 | 0.003401929 | UP |
| Test 2 | SNORA10 | 0.500134561 | 0.002159221 | UP |
| Test 2 | SNORA14B | 0.45085888 | 0.041534114 | UP |
| Test 2 | SNORA16A | 0.800445194 | 3.37E−05 | UP |
| Test 2 | SNORA21 | 0.715146141 | 0.000691404 | UP |
| Test 2 | SNORA23 | 0.496231233 | 0.003562425 | UP |
| Test 2 | SNORA24 | 0.62246595 | 0.000620204 | UP |
| Test 2 | SNORA33 | 0.438137876 | 0.02535746 | UP |
| Test 2 | SNORA34 | 0.656213162 | 0.000524629 | UP |
| Test 2 | SNORA38 | 0.634158916 | 0.003980579 | UP |
| Test 2 | SNORA49 | 0.391208768 | 0.046528283 | UP |
| Test 2 | SNORA50 | 0.501154595 | 0.004445349 | UP |
| Test 2 | SNORA52 | 0.691692913 | 0.000529232 | UP |
| Test 2 | SNORA57 | 0.670436835 | 0.000193375 | UP |
| Test 2 | SNORA6 | 0.370693768 | 0.043296102 | UP |
| Test 2 | SNORA62 | 0.442287413 | 0.013819155 | UP |
| Test 2 | SNORA63 | 0.532547036 | 0.003373425 | UP |
| Test 2 | SNORA65 | 0.448397229 | 0.026519056 | UP |
| Test 2 | SNORA67 | 0.441883998 | 0.017176358 | UP |
| Test 2 | SNORA68 | 0.857238018 | 4.58E−06 | UP |
| Test 2 | SNORA71A | 0.77835002 | 6.58E−05 | UP |
| Test 2 | SNORA71B | 0.529565961 | 0.004265337 | UP |
| Test 2 | SNORA71C | 0.495048141 | 0.007860449 | UP |
| Test 2 | SNORA71D | 0.435120855 | 0.018190672 | UP |
| Test 2 | SNORA74A | 0.610355277 | 0.004621969 | UP |
| Test 2 | SNORA74B | 0.645103623 | 0.004736561 | UP |
| Test 2 | SNORA7B | 0.492973554 | 0.010228164 | UP |
| Test 2 | SNORA84 | 0.625894973 | 0.001979651 | UP |
| Test 2 | SNORA9 | 0.497227196 | 0.007127047 | UP |
| Test 2 | SNORD15A | 0.637610126 | 0.001369934 | UP |
| Test 2 | SNORD15B | 0.59085556 | 0.00058311 | UP |
| Test 2 | SNORD17 | 0.357567591 | 0.045043368 | UP |
| Test 2 | SNORD94 | 0.826225768 | 6.63E−05 | UP |
| Test 2 | SNRPD1 | 0.511252623 | 0.028348085 | UP |

TABLE 1-18

| | | | | |
|---|---|---|---|---|
| Test 2 | SNRPE | 0.569496878 | 0.00533683 | UP |
| Test 2 | SNRPF | 0.723093533 | 0.002784753 | UP |
| Test 2 | SNRPG | 0.533113185 | 0.003903621 | UP |
| Test 2 | SOS1 | 0.510197893 | 0.008126315 | UP |
| Test 2 | SPINK5 | 0.679695473 | 0.013783683 | UP |
| Test 2 | SPINK7 | 0.833537404 | 0.008934427 | UP |
| Test 2 | SPRED1 | 0.448131411 | 0.045688393 | UP |
| Test 2 | SPRRIA | 0.588650149 | 0.016152579 | UP |
| Test 2 | SPRR1B | 0.56105932 | 0.012468229 | UP |
| Test 2 | SPRR2D | 1.026630516 | 1.40E−05 | UP |
| Test 2 | SPRR2E | 0.812347856 | 0.000312759 | UP |
| Test 2 | SPRR2F | 0.6818015 | 0.01623415 | UP |
| Test 2 | SPRR3 | 1.262348143 | 0.010954072 | UP |
| Test 2 | SPTLC1 | 0.676124476 | 0.00023443 | UP |
| Test 2 | SPTLC2 | 0.466842018 | 0.021700887 | UP |
| Test 2 | SRD5A1 | 0.384823816 | 0.048171711 | UP |
| Test 2 | SRSF10 | 0.559315822 | 0.003436841 | UP |
| Test 2 | SSBP1 | 0.362932978 | 0.040546721 | UP |
| Test 2 | SSBP3 | 0.477696067 | 0.030247231 | UP |
| Test 2 | STAP2 | 0.595061813 | 0.020857904 | UP |
| Test 2 | SUMF2 | 0.520831633 | 0.008329324 | UP |
| Test 2 | SYBU | 0.795756793 | 0.010109319 | UP |
| Test 2 | TADA2B | 0.650173529 | 0.00745433 | UP |
| Test 2 | TCEAl | 0.41056745 | 0.030342599 | UP |
| Test 2 | TCHH | 1.178806419 | 8.10E−05 | UP |
| Test 2 | TCHHL1 | 1.300728249 | 5.53E−05 | UP |
| Test 2 | TFAP2C | 0.503894804 | 0.049079738 | UP |
| Test 2 | TFIP11 | 0.475273026 | 0.013898256 | UP |
| Test 2 | TGM3 | 1.042385225 | 0.004077755 | UP |
| Test 2 | THOC7 | 0.413919226 | 0.049112511 | UP |
| Test 2 | TIA1 | 0.490818268 | 0.006610946 | UP |
| Test 2 | TM4SF1 | 0.476103934 | 0.034341158 | UP |
| Test 2 | TM4SF19 | 0.971617642 | 0.000218413 | UP |
| Test 2 | TMEM179B | 0.389161719 | 0.035487717 | UP |
| Test 2 | TMEM45B | 0.547241184 | 0.03914833 | UP |
| Test 2 | TMEM60 | 0.408255653 | 0.033088543 | UP |
| Test 2 | TPRG1 | 0.86840816 | 0.011100172 | UP |
| Test 2 | TRAF4 | 0.577432006 | 0.023791558 | UP |
| Test 2 | TRAK2 | 0.711752054 | 0.000495145 | UP |
| Test 2 | TRAPPC2L | 0.909322771 | 1.74E−05 | UP |
| Test 2 | TRMT6 | 0.549132145 | 0.002369651 | UP |

TABLE 1-19

| | | | | |
|---|---|---|---|---|
| Test 2 | TRPT1 | 0.438418122 | 0.021480307 | UP |
| Test 2 | TSC2 | 0.397964507 | 0.021089712 | UP |
| Test 2 | TSPO | 0.345346267 | 0.025202467 | UP |
| Test 2 | TSR1 | 0.558146659 | 0.014222557 | UP |
| Test 2 | TTPAL | 0.459459863 | 0.034963074 | UP |
| Test 2 | TUBB2A | 0.525704007 | 0.020268788 | UP |
| Test 2 | TWF1 | 0.373452154 | 0.047899723 | UP |
| Test 2 | TXNDC17 | 0.714879216 | 0.000129111 | UP |
| Test 2 | TXNRD1 | 1.099101729 | 0.001183883 | UP |
| Test 2 | UBE2L3 | 0.526673618 | 0.000211555 | UP |
| Test 2 | UBL3 | 0.474834314 | 0.007289944 | UP |
| Test 2 | UBL5 | 0.376245303 | 0.005590702 | UP |
| Test 2 | UCHL3 | 0.538513933 | 0.012719666 | UP |
| Test 2 | UGP2 | 0.412622582 | 0.011054428 | UP |
| Test 2 | UNC50 | 0.424239923 | 0.033163478 | UP |
| Test 2 | UQCR10 | 0.337398522 | 0.0413842 | UP |
| Test 2 | UQCRH | 0.346746063 | 0.030618555 | UP |
| Test 2 | UTP6 | 0.477781959 | 0.037563094 | UP |
| Test 2 | VASN | 0.580611332 | 0.028789273 | UP |
| Test 2 | VPS4A | 0.509642105 | 0.028041104 | UP |
| Test 2 | VSIG8 | 0.679661792 | 0.024720039 | UP |
| Test 2 | WDR60 | 0.685964377 | 0.004520911 | UP |
| Test 2 | WDR61 | 0.451460615 | 0.04499615 | UP |
| Test 2 | WFDC12 | 0.911124303 | 0.015496389 | UP |
| Test 2 | WFDC5 | 0.630799027 | 0.035863131 | UP |
| Test 2 | WIBG | 0.471704215 | 0.039582096 | UP |
| Test 2 | WWTR1 | 0.715620692 | 0.004105629 | UP |
| Test 2 | XPOT | 0.468281083 | 0.045788227 | UP |
| Test 2 | YTHDF1 | 0.386322627 | 0.038012885 | UP |
| Test 2 | YTHDF2 | 0.62946844 | 0.002443494 | UP |
| Test 2 | ZFAND2A | 0.450586185 | 0.018177494 | UP |
| Test 2 | ZNF259 | 0.481902886 | 0.003455703 | UP |

TABLE 1-20

| | | | | |
|---|---|---|---|---|
| Test 2 | ABTB1 | −0.546411505 | 0.018144091 | DOWN |
| Test 2 | ADAM8 | −0.513306321 | 0.035447376 | DOWN |
| Test 2 | ADORA2A | −0.81885306 | 0.002331507 | DOWN |
| Test 2 | AGTRAP | −0.538277133 | 0.006210892 | DOWN |
| Test 2 | AGXT2L2 | −0.484250819 | 0.016167126 | DOWN |
| Test 2 | AHCYL1 | −0.414076682 | 0.031287665 | DOWN |
| Test 2 | ALPL | −0.732971219 | 0.037662124 | DOWN |
| Test 2 | ANKRD12 | −0.584058465 | 0.022801499 | DOWN |
| Test 2 | ANKRD17 | −0.405544382 | 0.007522217 | DOWN |
| Test 2 | ANKRD27 | −0.457996379 | 0.047461771 | DOWN |
| Test 2 | AP1G1 | −0.353067009 | 0.020214307 | DOWN |
| Test 2 | APH1A | −0.525725945 | 0.014352743 | DOWN |
| Test 2 | ARF1 | −0.23559285 | 0.007275376 | DOWN |
| Test 2 | ARF5 | −0.359483978 | 0.007087883 | DOWN |
| Test 2 | ARHGAP30 | −0.671059506 | 0.003502527 | DOWN |
| Test 2 | ARHGEF2 | −0.365713515 | 0.046292884 | DOWN |
| Test 2 | ARID3A | −0.5595524 | 0.020763141 | DOWN |
| Test 2 | ARL5B | −0.374255491 | 0.032148928 | DOWN |
| Test 2 | ARPC2 | −0.301380741 | 0.007856807 | DOWN |
| Test 2 | ATG2A | −0.491971315 | 0.004958653 | DOWN |
| Test 2 | ATHL1 | −0.839020018 | 0.002709554 | DOWN |
| Test 2 | ATP13A3 | −0.343948111 | 0.034155817 | DOWN |
| Test 2 | ATP6VOC | −0.528286893 | 0.000408234 | DOWN |
| Test 2 | ATP6VOD1 | −0.287114942 | 0.039585219 | DOWN |
| Test 2 | AURKAIP1 | −0.502619683 | 0.00958826 | DOWN |
| Test 2 | BAKI | −0.487024115 | 0.040014771 | DOWN |
| Test 2 | BAP1 | −0.504388246 | 0.01853754 | DOWN |
| Test 2 | BMP2K | −0.61467808 | 0.039213696 | DOWN |
| Test 2 | BRD2 | −0.365257538 | 0.006933573 | DOWN |
| Test 2 | BSDC1 | −0.374730519 | 0.046154207 | DOWN |
| Test 2 | C15orf38 | −0.715339518 | 0.016230138 | DOWN |
| Test 2 | C17orf107 | −0.638711636 | 0.031899208 | DOWN |
| Test 2 | C22orf13 | −0.556229527 | 0.002139299 | DOWN |
| Test 2 | CAMKID | −0.363332672 | 0.048425091 | DOWN |
| Test 2 | CANT1 | −0.619432538 | 0.005612133 | DOWN |
| Test 2 | CASP9 | −0.391150283 | 0.046236587 | DOWN |
| Test 2 | CCDC28A | −0.325084738 | 0.039941927 | DOWN |
| Test 2 | CCDC9 | −0.381074881 | 0.032466986 | DOWN |
| Test 2 | CCL3 | −0.647010847 | 0.028352908 | DOWN |
| Test 2 | CCNI | −0.335231908 | 0.027018957 | DOWN |
| Test 2 | CCRL2 | −0.588173323 | 0.040593791 | DOWN |

TABLE 1-21

| | | | | |
|---|---|---|---|---|
| Test 2 | CD63 | −0.445497418 | 0.004230269 | DOWN |
| Test 2 | CD83 | −0.526594744 | 0.029159029 | DOWN |
| Test 2 | CD97 | −0.76193437 | 0.005030014 | DOWN |
| Test 2 | CDC42SE1 | −0.371810977 | 0.009492497 | DOWN |
| Test 2 | CDKNIA | −0.409759913 | 0.000877116 | DOWN |
| Test 2 | CFL1 | −0.235920095 | 0.045298064 | DOWN |
| Test 2 | CHD2 | −0.534365582 | 0.01031488 | DOWN |
| Test 2 | CIC | −0.780568746 | 0.001746158 | DOWN |
| Test 2 | CNN2 | −0.478206967 | 0.045041795 | DOWN |
| Test 2 | CRLF3 | −0.470829152 | 0.018171553 | DOWN |
| Test 2 | CSF1 | −0.859593671 | 0.001648257 | DOWN |
| Test 2 | CSF2RB | −0.537088027 | 0.047037042 | DOWN |
| Test 2 | CSRNP1 | −0.702210165 | 0.000255859 | DOWN |
| Test 2 | CTBP2 | −0.482865997 | 0.039884842 | DOWN |
| Test 2 | CTDSP2 | −0.549610429 | 0.000270136 | DOWN |
| Test 2 | CXCR4 | −0.628204085 | 0.020358444 | DOWN |
| Test 2 | CYTH1 | −0.39211801 | 0.021273331 | DOWN |
| Test 2 | DBNL | −0.361312286 | 0.009931476 | DOWN |
| Test 2 | DCAF11 | −0.6326978 | 0.001569843 | DOWN |
| Test 2 | DENND5A | −0.563683623 | 0.011198898 | DOWN |
| Test 2 | DESI1 | −0.476146449 | 0.025976354 | DOWN |
| Test 2 | DGAT1 | −0.486566263 | 0.018563147 | DOWN |
| Test 2 | DNM2 | −0.613664304 | 0.034363766 | DOWN |
| Test 2 | DOTIL | −0.437381988 | 0.033054977 | DOWN |
| Test 2 | DUSP1 | −0.456302054 | 0.039191808 | DOWN |
| Test 2 | DUSP2 | −0.511851781 | 0.011855554 | DOWN |
| Test 2 | DUSP3 | −0.539021616 | 0.003981359 | DOWN |
| Test 2 | ECD | −0.44764865 | 0.027268857 | DOWN |
| Test 2 | EFHD2 | −0.472250359 | 0.032124386 | DOWN |
| Test 2 | EFR3A | −0.357748504 | 0.035446993 | DOWN |
| Test 2 | EGR2 | −0.299185803 | 0.033982561 | DOWN |
| Test 2 | EGR3 | −0.600613853 | 0.002514735 | DOWN |
| Test 2 | EIF2C4 | −0.507646506 | 0.040582559 | DOWN |
| Test 2 | EIF4EBP2 | −0.415388743 | 0.02944828 | DOWN |
| Test 2 | ELF1 | −0.414700395 | 0.046718101 | DOWN |
| Test 2 | EMP3 | −0.56727553 | 0.012074994 | DOWN |
| Test 2 | EPS15L1 | −0.43465134 | 0.021095706 | DOWN |
| Test 2 | FAM100B | −0.396320013 | 0.007456757 | DOWN |
| Test 2 | FAM193B | −0.816752521 | 0.006550877 | DOWN |
| Test 2 | FAM210A | −0.415999641 | 0.043646384 | DOWN |
| Test 2 | FAM32A | −0.441434954 | 0.00711492 | DOWN |

TABLE 1-22

| | | | | |
|---|---|---|---|---|
| Test 2 | FAM53C | −0.414646652 | 0.043215387 | DOWN |
| Test 2 | FBXO11 | −0.587567686 | 0.033095048 | DOWN |
| Test 2 | FCGRT | −0.593104023 | 0.019455764 | DOWN |
| Test 2 | FGR | −0.573604518 | 0.025328892 | DOWN |
| Test 2 | FLNA | −0.503978457 | 0.020310777 | DOWN |
| Test 2 | FNIP1 | −0.559259947 | 0.024530856 | DOWN |
| Test 2 | FOSB | −1.091622363 | 0.000150044 | DOWN |
| Test 2 | FOSL2 | −0.741546633 | 0.000377548 | DOWN |
| Test 2 | FOXN3 | −0.354637174 | 0.046745405 | DOWN |
| Test 2 | FOXO4 | −0.4667223 | 0.043783003 | DOWN |
| Test 2 | FURIN | −0.459105715 | 0.001341881 | DOWN |
| Test 2 | FZRI | −0.364147622 | 0.028337243 | DOWN |
| Test 2 | GABARAPLI | −0.55597523 | 0.006898537 | DOWN |
| Test 2 | GADD45B | −0.470481527 | 0.001471104 | DOWN |
| Test 2 | GAPVD1 | −0.410369844 | 0.017202036 | DOWN |
| Test 2 | GATAD2A | −0.427073771 | 0.023639602 | DOWN |
| Test 2 | GGA1 | −0.396427118 | 0.011108895 | DOWN |
| Test 2 | GLA | −0.432386163 | 0.046129953 | DOWN |
| Test 2 | GMIP | −0.439255443 | 0.025650159 | DOWN |
| Test 2 | GNB1 | −0.31847551 | 0.015581144 | DOWN |
| Test 2 | GNB2 | −0.319721149 | 0.049773636 | DOWN |
| Test 2 | GPR108 | −0.441903322 | 0.042000281 | DOWN |
| Test 2 | GPX1 | −0.419015476 | 0.012872784 | DOWN |
| Test 2 | GRAMDIA | −0.932263643 | 1.44E−05 | DOWN |
| Test 2 | GRK6 | −0.654615424 | 0.008370268 | DOWN |
| Test 2 | GRN | −0.551500985 | 0.014350263 | DOWN |
| Test 2 | GTPBP1 | −0.403503204 | 0.015278622 | DOWN |
| Test 2 | HEXIMI | −0.365986502 | 0.049415504 | DOWN |
| Test 2 | HIPK3 | −0.463202659 | 0.018014847 | DOWN |
| Test 2 | HLA.A | −1.236792406 | 0.020861464 | DOWN |
| Test 2 | HLX | −0.624323089 | 0.020911612 | DOWN |
| Test 2 | HSPA4 | −0.59623271 | 0.022837699 | DOWN |
| Test 2 | IDS | −0.240881411 | 0.028746962 | DOWN |
| Test 2 | IER3 | −0.287520838 | 0.017217201 | DOWN |
| Test 2 | IMPDH1 | −0.610405695 | 0.010620152 | DOWN |
| Test 2 | INO80D | −0.37547378 | 0.007394184 | DOWN |
| Test 2 | INPP5K | −0.423372501 | 0.028673174 | DOWN |
| Test 2 | IQSEC1 | −0.390427136 | 0.017062257 | DOWN |
| Test 2 | IRAK2 | −0.658169882 | 0.010698571 | DOWN |
| Test 2 | IRS2 | −0.420496894 | 0.042158917 | DOWN |
| Test 2 | ISCU | −0.287869125 | 0.027433296 | DOWN |

TABLE 1-23

| | | | | |
|---|---|---|---|---|
| Test 2 | ISG20L2 | −0.294870703 | 0.042571274 | DOWN |
| Test 2 | ITGA5 | −0.468532816 | 0.03498499 | DOWN |
| Test 2 | ITGAM | −0.527588792 | 0.029840274 | DOWN |
| Test 2 | ITGAX | −0.64770333 | 0.014582029 | DOWN |
| Test 2 | JARID2 | −0.490688966 | 0.018106974 | DOWN |
| Test 2 | JUNB | −0.293143391 | 0.04364956 | DOWN |
| Test 2 | KAT5 | −0.418414536 | 0.014457718 | DOWN |
| Test 2 | KDM6B | −0.692508948 | 0.021431857 | DOWN |
| Test 2 | KIAA0232 | −0.381744969 | 0.033575438 | DOWN |
| Test 2 | KIAA0513 | −0.533596569 | 0.029885073 | DOWN |
| Test 2 | KLF2 | −0.643760892 | 0.043067951 | DOWN |
| Test 2 | KLF6 | −0.548841797 | 0.000827261 | DOWN |
| Test 2 | KLHL2 | −0.943508712 | 0.003900525 | DOWN |
| Test 2 | LATS2 | −0.464918389 | 0.023816082 | DOWN |
| Test 2 | LILRB2 | −0.517795974 | 0.044488235 | DOWN |
| Test 2 | LIMSI | −0.49409161 | 0.012867015 | DOWN |
| Test 2 | LITAF | −0.329270813 | 0.029150473 | DOWN |
| Test 2 | LOC283070 | −0.442909512 | 0.028945909 | DOWN |
| Test 2 | LPAR2 | −0.547607501 | 0.027604213 | DOWN |
| Test 2 | LPCAT1 | −0.832260386 | 0.002421822 | DOWN |
| Test 2 | LSP1 | −0.601135718 | 0.002264273 | DOWN |
| Test 2 | LTBR | −0.485771016 | 0.02744313 | DOWN |
| Test 2 | MAFI | −0.526440437 | 0.015395944 | DOWN |
| Test 2 | MAN2A1 | −0.652423245 | 0.045009558 | DOWN |
| Test 2 | MAP4K4 | −0.364791966 | 0.026304596 | DOWN |
| Test 2 | MAP7D1 | −0.445039844 | 0.012145517 | DOWN |
| Test 2 | MAPKAPK2 | −0.32646322 | 0.018751928 | DOWN |
| Test 2 | MECP2 | −0.759034568 | 0.000179172 | DOWN |
| Test 2 | MEF2D | −0.48601618 | 0.004466755 | DOWN |
| Test 2 | METRNL | −0.31396947 | 0.014421188 | DOWN |
| Test 2 | MGEA5 | −0.417769836 | 0.00311289 | DOWN |
| Test 2 | MIDN | −0.412523462 | 0.032312233 | DOWN |
| Test 2 | MKNK2 | −0.468504375 | 0.006063396 | DOWN |
| Test 2 | MLF2 | −0.522610118 | 0.00717405 | DOWN |
| Test 2 | MLLT6 | −0.562801463 | 0.012870027 | DOWN |
| Test 2 | MMP25 | −0.607156567 | 0.040654743 | DOWN |
| Test 2 | MTHFS | −0.620132887 | 0.008704266 | DOWN |
| Test 2 | MTMR14 | −0.500904907 | 0.027113371 | DOWN |
| Test 2 | MYADM | −0.532524278 | 0.032770779 | DOWN |
| Test 2 | MY09B | −0.465760356 | 0.012683393 | DOWN |
| Test 2 | NAA50 | −0.333033539 | 0.024091716 | DOWN |

TABLE 1-24

| | | | | |
|---|---|---|---|---|
| Test 2 | NABI | −0.41631867 | 0.034705696 | DOWN |
| Test 2 | NAGK | −0.400938958 | 0.039418511 | DOWN |
| Test 2 | NCF1B | −0.632988161 | 0.032521317 | DOWN |
| Test 2 | NCF1C | −0.564958434 | 0.023648103 | DOWN |
| Test 2 | NCOA1 | −0.35268749 | 0.025504935 | DOWN |
| Test 2 | NFKB2 | −0.686225906 | 0.006490871 | DOWN |
| Test 2 | NFKBIB | −0.417375331 | 0.020054211 | DOWN |
| Test 2 | NFKBID | −0.579020216 | 0.039512351 | DOWN |
| Test 2 | NINJ1 | −0.666521399 | 0.007758421 | DOWN |
| Test 2 | NLRC5 | −0.518289968 | 0.04615466 | DOWN |
| Test 2 | NOTCH2NL | −0.380526931 | 0.002073988 | DOWN |
| Test 2 | NRIP1 | −1.322958378 | 0.002632999 | DOWN |
| Test 2 | NUMB | −0.494870767 | 0.00364152 | DOWN |
| Test 2 | OGFR | −0.457666083 | 0.021935407 | DOWN |
| Test 2 | OS9 | −0.472649391 | 0.045293803 | DOWN |
| Test 2 | PAN3 | −0.490759714 | 0.037403044 | DOWN |
| Test 2 | PATL1 | −0.425494161 | 0.039431793 | DOWN |
| Test 2 | PCBP1 | −0.176849095 | 0.0308842 | DOWN |
| Test 2 | PDPK1 | −0.351764848 | 0.030720043 | DOWN |
| Test 2 | PERI | −0.520214927 | 0.038720114 | DOWN |
| Test 2 | PFKFB3 | −0.371937997 | 0.012048698 | DOWN |
| Test 2 | PHF1 | −0.509490418 | 0.018640047 | DOWN |
| Test 2 | PIK3AP1 | −0.630445334 | 0.004184868 | DOWN |
| Test 2 | PIK3R5 | −0.612446475 | 0.004720621 | DOWN |
| Test 2 | PIM3 | −0.467577174 | 0.002878904 | DOWN |
| Test 2 | PITPNA | −0.474470422 | 0.00241514 | DOWN |
| Test 2 | PLAU | −0.65031011 | 0.029875395 | DOWN |
| Test 2 | PLEKHB2 | −0.305583054 | 0.044277802 | DOWN |
| Test 2 | PLEKHM3 | −0.368794416 | 0.029647876 | DOWN |
| Test 2 | PLIN5 | −0.676960181 | 0.015080446 | DOWN |
| Test 2 | PPP1R15A | −0.418072337 | 0.005793369 | DOWN |
| Test 2 | PPP1R18 | −0.506261932 | 0.019385963 | DOWN |
| Test 2 | PPP2R5C | −0.471507643 | 0.029204209 | DOWN |
| Test 2 | PPP4R1 | −0.578649371 | 0.006631286 | DOWN |
| Test 2 | PRR14 | −0.46051795 | 0.0377872 | DOWN |
| Test 2 | PRR24 | −0.39469883 | 0.038397986 | DOWN |
| Test 2 | PRRC2C | −0.383243267 | 0.047022553 | DOWN |
| Test 2 | PTGER4 | −0.467431527 | 0.024894507 | DOWN |
| Test 2 | PTK2B | −0.429404802 | 0.005990901 | DOWN |
| Test 2 | PTTG1IP | −0.481590933 | 0.044232468 | DOWN |
| Test 2 | RAB11FIP1 | −0.22457918 | 0.041085596 | DOWN |

TABLE 1-25

| | | | | |
|---|---|---|---|---|
| Test 2 | RAB20 | −0.640257296 | 0.027879149 | DOWN |
| Test 2 | RAB5C | −0.399368933 | 0.007878983 | DOWN |
| Test 2 | RALGDS | −0.524141036 | 0.022754244 | DOWN |
| Test 2 | RAP2C | −0.429682327 | 0.044030988 | DOWN |
| Test 2 | RBCK1 | −0.576800376 | 0.038892289 | DOWN |
| Test 2 | RBM39 | −0.42348233 | 0.016771793 | DOWN |
| Test 2 | RBM4 | −0.319253158 | 0.022873847 | DOWN |
| Test 2 | RELA | −0.484272048 | 0.003538465 | DOWN |
| Test 2 | RGS19 | −0.569964421 | 0.00660173 | DOWN |
| Test 2 | RHBDD2 | −0.351071124 | 0.041709589 | DOWN |
| Test 2 | RHEB | −0.372307356 | 0.006704257 | DOWN |
| Test 2 | RHOA | −0.299449889 | 0.004939206 | DOWN |
| Test 2 | RHOB | −0.626515052 | 0.00782575 | DOWN |
| Test 2 | RILPL2 | −0.751957556 | 0.011595227 | DOWN |
| Test 2 | RNASEK | −0.203072703 | 0.046581317 | DOWN |
| Test 2 | RNF13 | −0.445901036 | 0.045657953 | DOWN |
| Test 2 | RNF41 | −0.405181229 | 0.01043338 | DOWN |
| Test 2 | RTN4 | −0.45443723 | 0.003045171 | DOWN |
| Test 2 | RXRA | −0.438666828 | 0.003686277 | DOWN |
| Test 2 | RYBP | −0.450501153 | 0.006086994 | DOWN |
| Test 2 | SBNO2 | −0.549309601 | 0.010378319 | DOWN |
| Test 2 | SCYL1 | −0.410832924 | 0.012148609 | DOWN |
| Test 2 | SDE2 | −0.345790438 | 0.046190193 | DOWN |
| Test 2 | SEC22B | −0.255374419 | 0.036042427 | DOWN |
| Test 2 | SEMA6B | −0.5268738 | 0.041383614 | DOWN |
| Test 2 | SERINC1 | −0.54365295 | 0.011959311 | DOWN |
| Test 2 | SERP1 | −0.296323781 | 0.027306968 | DOWN |
| Test 2 | SF3B2 | −0.309131592 | 0.04838585 | DOWN |
| Test 2 | SH3BP5 | −0.457133655 | 0.025096704 | DOWN |
| Test 2 | SHISA5 | −0.703515786 | 0.03999053 | DOWN |
| Test 2 | SIPA1 | −0.534045003 | 0.043975734 | DOWN |
| Test 2 | SIRPA | −0.367127888 | 0.004462404 | DOWN |
| Test 2 | SLC11A1 | −0.583530702 | 0.045040558 | DOWN |
| Test 2 | SLC15A3 | −0.482099344 | 0.041303655 | DOWN |
| Test 2 | SLC16A3 | −0.590518437 | 0.027963972 | DOWN |
| Test 2 | SLC25A6 | −0.401411655 | 0.018627665 | DOWN |
| Test 2 | SLC3A2 | −0.593512339 | 0.004761774 | DOWN |
| Test 2 | SLC43A2 | −0.717518005 | 0.003148231 | DOWN |
| Test 2 | SLC44A2 | −0.366045357 | 0.026415807 | DOWN |
| Test 2 | SLC6A6 | −0.696190042 | 0.0043814 | DOWN |
| Test 2 | SLC9A8 | −0.589473162 | 0.029051064 | DOWN |

TABLE 1-26

| | | | | |
|---|---|---|---|---|
| Test 2 | SLED1 | −0.693849284 | 0.028188168 | DOWN |
| Test 2 | SMG1P1 | −0.574995261 | 0.020839424 | DOWN |
| Test 2 | SPHK1 | −0.61543386 | 0.004539302 | DOWN |
| Test 2 | SQSTM1 | −0.257715842 | 0.046959382 | DOWN |
| Test 2 | SREBF2 | −0.69315397 | 0.006195022 | DOWN |
| Test 2 | SRRM2 | −0.44624329 | 0.010131156 | DOWN |
| Test 2 | SRXN1 | −0.393283821 | 0.048331481 | DOWN |
| Test 2 | STK40 | −0.443882447 | 0.001414866 | DOWN |
| Test 2 | STX11 | −0.537977782 | 0.004822597 | DOWN |
| Test 2 | STX3 | −0.52789925 | 0.015799785 | DOWN |
| Test 2 | STX6 | −0.616930664 | 0.036164799 | DOWN |
| Test 2 | STXBP2 | −0.3689503 | 0.021350599 | DOWN |
| Test 2 | SUPT6H | −0.353757744 | 0.027853669 | DOWN |
| Test 2 | TAF10 | −0.442411568 | 0.003533838 | DOWN |
| Test 2 | TANK | −0.545432207 | 0.031687959 | DOWN |
| Test 2 | TCF25 | −0.409012121 | 0.024330453 | DOWN |
| Test 2 | TCIRG1 | −0.614475203 | 0.007539619 | DOWN |
| Test 2 | TM9SF4 | −0.38535586 | 0.04829699 | DOWN |
| Test 2 | TMBIM6 | −0.297865078 | 0.008470387 | DOWN |
| Test 2 | TMEM123 | −0.343267776 | 0.009094089 | DOWN |
| Test 2 | TMEM167B | −0.359258554 | 0.007761458 | DOWN |
| Test 2 | TMEM183A | −0.34494503 | 0.03937267 | DOWN |
| Test 2 | TMEM66 | −0.271219311 | 0.031611284 | DOWN |
| Test 2 | TMX4 | −0.900650969 | 0.002108645 | DOWN |
| Test 2 | TNFAIP2 | −0.568352841 | 0.021862206 | DOWN |
| Test 2 | TNFAIP3 | −0.665510696 | 0.007064788 | DOWN |
| Test 2 | TNFRSF14 | −0.543798981 | 0.014449198 | DOWN |
| Test 2 | TOM1 | −0.352436371 | 0.011291364 | DOWN |
| Test 2 | TP53INP2 | −0.423916841 | 0.049618263 | DOWN |
| Test 2 | TRAPPC5 | −0.329107904 | 0.045571636 | DOWN |
| Test 2 | TSPAN13 | −0.440929311 | 0.032283555 | DOWN |
| Test 2 | TTYH3 | −0.4874383 | 0.043267217 | DOWN |
| Test 2 | UBAP2L | −0.542649397 | 0.002387811 | DOWN |
| Test 2 | UBE2D3 | −0.456380993 | 2.47E−05 | DOWN |
| Test 2 | UBR4 | −0.760894686 | 0.002561835 | DOWN |
| Test 2 | UCP2 | −0.552795075 | 0.002082641 | DOWN |
| Test 2 | UPF1 | −0.336863796 | 0.026989745 | DOWN |
| Test 2 | USB1 | −0.406612823 | 0.033709698 | DOWN |
| Test 2 | USF2 | −0.486370326 | 0.00456431 | DOWN |
| Test 2 | WBP2 | −0.50639192 | 0.002504203 | DOWN |
| Test 2 | WDR82 | −0.410119457 | 0.031724832 | DOWN |

TABLE 1-27

| | | | | |
|---|---|---|---|---|
| Test 2 | XPO6 | −0.597709348 | 0.046102314 | DOWN |
| Test 2 | YPEL5 | −0.287506377 | 0.038298209 | DOWN |
| Test 2 | ZC3H12A | −0.511217461 | 0.009065486 | DOWN |
| Test 2 | ZFP36 | −0.468506172 | 0.020859393 | DOWN |
| Test 2 | ZMIZ1 | −0.651337052 | 0.00341487 | DOWN |
| Test 2 | ZNFX1 | −0.447727612 | 0.044337198 | DOWN |
| Test 2 | ZZEF1 | −0.356247435 | 0.015261504 | DOWN |

A biological process (BP) and a KEGG pathway were searched for by gene ontology (GO) enrichment analysis by using the public database STRING. As a result, 30 and 39 KEGG pathways related to the gene group with increased or decreased expression in the PD patients were obtained in Test 1 and Test 2, respectively, and the term hsa05012 (Parkinson's disease) which indicates Parkinson's disease was found to be included in both the tests (Tables 2-1 and 2-2).

TABLE 2-1

| Test | Regulation | ID | Description | FDR |
|---|---|---|---|---|
| Test 1 | UP | hsa00190 | Oxidative phosphorylation | 1.73E−08 |
| Test 1 | UP | hsa04932 | Non-alcoholic fatty liver | 4.79E−07 |
| | | | disease (NAFLD) | |
| Test 1 | UP | hsa05012 | Parkinson's disease | 3.00E−06 |
| Test 1 | UP | hsa05016 | Huntington's disease | 3.00E−06 |
| Test 1 | UP | hsa05010 | Alzheimer's disease | 7.01E−06 |
| Test 1 | UP | hsa04714 | Thermogenesis | 7.19E−06 |
| Test 1 | UP | hsaOHOO | Metabolic pathways | 0.00028 |
| Test 1 | UP | hsa04260 | Cardiac muscle contraction | 0.00092 |
| Test 1 | UP | hsa03050 | Proteasome | 0.0014 |
| Test 1 | UP | hsa04723 | Retrograde endocannabinoid signaling | 0.0142 |
| Test 1 | UP | hsa05219 | Bladder cancer | 0.0174 |
| Test 1 | UP | hsa05169 | Epstein−Barr virus infection | 0.0374 |
| Test 1 | DOWN | hsa03010 | Ribosome | 1.27E−13 |
| Test 1 | DOWN | hsa04062 | Chemokine signaling pathway | 0.00017 |
| Test 1 | DOWN | hsa04144 | Endocytosis | 0.0065 |
| Test 1 | DOWN | hsa05132 | Salmonella infection | 0.0065 |
| Test 1 | DOWN | hsa05203 | Viral carcinogenesis | 0.0091 |
| Test 1 | DOWN | hsa04670 | Leukocyte transendothelial migration | 0.0114 |
| Test 1 | DOWN | hsa00061 | Fatty acid biosynthesis | 0.0139 |
| Test 1 | DOWN | hsa04014 | Ras signaling pathway | 0.0139 |
| Test 1 | DOWN | hsa05130 | Pathogenic Escherichia coli infection | 0.0139 |
| Test 1 | DOWN | hsa05100 | Bacterial invasion of epithelial cells | 0.0191 |
| Test 1 | DOWN | hsa05200 | Pathways in cancer | 0.0191 |
| Test 1 | DOWN | hsa05211 | Renal cell carcinoma | 0.0191 |
| Test 1 | DOWN | hsa04360 | Axon guidance | 0.0249 |
| Test 1 | DOWN | hsa04666 | Fc gamma R−mediated phagocytosis | 0.029 |
| Test 1 | DOWN | hsa05205 | Proteoglycans in cancer | 0.0328 |
| Test 1 | DOWN | hsa04066 | HIF−1 signaling pathway | 0.0329 |
| Test 1 | DOWN | hsa04810 | Regulation of actin cytoskeleton | 0.0344 |
| Test 1 | DOWN | hsa04722 | Neurotrophin signaling pathway | 0.0461 |

TABLE 2-2

| Test 2 | UP | hsa03010 | Ribosome | 4.70E−17 |
|---|---|---|---|---|
| Test 2 | UP | hsa04714 | Thermogenesis | 1.98E−05 |
| Test 2 | UP | hsa05016 | Huntington's disease | 0.00022 |
| Test 2 | UP | hsa00190 | Oxidative phosphorylation | 0.00034 |
| Test 2 | UP | hsa05010 | Alzheimer's disease | 0.00034 |
| Test 2 | UP | hsa05012 | Parkinson's disease | 0.00056 |
| Test 2 | UP | hsa00280 | Valine, leucine and isoleucine degradation | 0.003 |
| Test 2 | UP | hsa03040 | Spliceosome | 0.0094 |
| Test 2 | UP | hsaOHOO | Metabolic pathways | 0.0188 |
| Test 2 | DOWN | hsa04142 | Lysosome | 0.0035 |
| Test 2 | DOWN | hsa05152 | Tuberculosis | 0.0035 |
| Test 2 | DOWN | hsa04072 | Phospholipase D signaling pathway | 0.0064 |
| Test 2 | DOWN | hsa04144 | Endocytosis | 0.0064 |
| Test 2 | DOWN | hsa04380 | Osteoclast differentiation | 0.0064 |
| Test 2 | DOWN | hsa05203 | Viral carcinogenesis | 0.0064 |
| Test 2 | DOWN | hsa05134 | Legionellosis | 0.0069 |
| Test 2 | DOWN | hsa04062 | Chemokine signaling pathway | 0.013 |
| Test 2 | DOWN | hsa05167 | Kaposi’s sarcoma-associated | 0.013 |
| | | | herpesvirus infection | |
| Test 2 | DOWN | hsa05223 | Non-small cell lung cancer | 0.0131 |
| Test 2 | DOWN | hsa04151 | PI3K-Akt signaling pathway | 0.0168 |
| Test 2 | DOWN | hsa05212 | Pancreatic cancer | 0.019 |
| Test 2 | DOWN | hsa05202 | Transcriptional misregulation in cancer | 0.0194 |
| Test 2 | DOWN | hsa04130 | SNARE interactions in vesicular transport | 0.0296 |
| Test 2 | DOWN | hsa05200 | Pathways in cancer | 0.0296 |
| Test 2 | DOWN | hsa05210 | Colorectal cancer | 0.0296 |
| Test 2 | DOWN | hsa05213 | Endometrial cancer | 0.0296 |
| Test 2 | DOWN | hsa04064 | NF-kappa B signaling pathway | 0.0316 |
| Test 2 | DOWN | hsa04140 | Autophagy - animal | 0.0316 |
| Test 2 | DOWN | hsa04218 | Cellular senescence | 0.0316 |
| Test 2 | DOWN | hsa04721 | Synaptic vesicle cycle | 0.0316 |
| Test 2 | DOWN | hsa05216 | Thyroid cancer | 0.0316 |
| Test 2 | DOWN | hsa05222 | Small cell lung cancer | 0.0316 |
| Test 2 | DOWN | hsa04068 | FoxO signaling pathway | 0.0342 |
| Test 2 | DOWN | hsa04371 | Apelin signaling pathway | 0.037 |
| Test 2 | DOWN | hsa04010 | MARK signaling pathway | 0.0408 |
| Test 2 | DOWN | hsa05133 | Pertussis | 0.0456 |
| Test 2 | DOWN | hsa05220 | Chronic myeloid leukemia | 0.0488 |
| Test 2 | DOWN | hsa04145 | Phagosome | 0.0495 |
| Test 2 | DOWN | hsa05110 | Vibrio cholerae infection | 0.0495 |

Previously reported literatures were checked about the relation to Parkinson's disease of the genes shown in Tables 1-1 to 1-27 described above which were differentially expressed in at least either Test 1 or Test 2. As a result, 21 genes shown in Table 3-1 among the genes differentially expressed in Test 1 and 92 genes shown in Tables 3-2 to 3-4 among the genes differentially expressed in Test 2 had not been reported so far on their relation to Parkinson's disease, demonstrating that these genes are capable of serving as novel markers for detecting Parkinson's disease. Genes indicated by boldface in the tables are common genes between Test 1 and Test 2.

TABLE 3-1

| Test | Symbol | Regulation |
|---|---|---|
| Test 1 | DUX4L4 | UP |
| Test 1 | GPBPILl | UP |
| Test 1 | KIAA0930 | UP |
| Test 1 | LOC100093631 | UP |
| Test 1 | LOC100506888 | UP |
| Test 1 | LOC349196 | UP |
| Test 1 | LOC401321 | UP |
| Test 1 | OR4F3 | UP |
| Test 1 | PQLC1 | UP |
| Test 1 | REXO1L2P | UP |
| Test 1 | SNORA16A | UP |
| Test 1 | SNORA24 | UP |
| Test 1 | SNORA43 | UP |
| Test 1 | SNORA50 | UP |
| Test 1 | SNORA8 | UP |
| Test 1 | TCEB3CL | UP |
| Test 1 | TTC9 | UP |
| Test 1 | USP17L5 | UP |
| Test 1 | USP17L6P | UP |
| Test 1 | ZNF33A | UP |
| Test 1 | SNORA53 | DOWN |

TABLE 3-2

| Test | Symbol | Regulation |
|---|---|---|
| Test 2 | ACSS3 | UP |
| Test 2 | C1orf52 | UP |
| Test 2 | C5orf43 | UP |
| Test 2 | COA1 | UP |
| Test 2 | FAM210B | UP |
| Test 2 | FAM25B | UP |
| Test 2 | FAM45A | UP |
| Test 2 | GTF3C6 | UP |
| Test 2 | HEATR5A | UP |
| Test 2 | IQCG | UP |
| Test 2 | ITPRIPL2 | UP |
| Test 2 | KIAA0240 | UP |
| Test 2 | KIAA1143 | UP |
| Test 2 | KRTAP1.5 | UP |
| Test 2 | KRTAP12.1 | UP |
| Test 2 | KRTAP12.2 | UP |
| Test 2 | KRTAP3.1 | UP |
| Test 2 | KRTAP5.3 | UP |
| Test 2 | LINC00675 | UP |
| Test 2 | LOC100505738 | UP |
| Test 2 | LOC550643 | UP |
| Test 2 | LOC646862 | UP |
| Test 2 | LRRC15 | UP |
| Test 2 | MICALCL | UP |
| Test 2 | PDE12 | UP |
| Test 2 | PINLYP | UP |
| Test 2 | REXO1L2P | UP |
| Test 2 | SCARNA12 | UP |
| Test 2 | SCARNA16 | UP |
| Test 2 | SCARNA6 | UP |
| Test 2 | SCARNA7 | UP |
| Test 2 | SF3B14 | UP |
| Test 2 | SLFN5 | UP |
| Test 2 | SLMO2 | UP |
| Test 2 | SMIM5 | UP |
| Test 2 | SNHG9 | UP |
| Test 2 | SNORA10 | UP |
| Test 2 | SNORA14B | UP |
| Test 2 | SNORA16A | UP |
| Test 2 | SNORA21 | UP |
| Test 2 | SNORA23 | UP |

TABLE 3-3

| | | |
|---|---|---|
| Test 2 | SNORA24 | UP |
| Test 2 | SNORA33 | UP |
| Test 2 | SNORA34 | UP |
| Test 2 | SNORA49 | UP |
| Test 2 | SNORA50 | UP |
| Test 2 | SNORA52 | UP |
| Test 2 | SNORA57 | UP |
| Test 2 | SNORA6 | UP |
| Test 2 | SNORA63 | UP |
| Test 2 | SNORA65 | UP |
| Test 2 | SNORA67 | UP |
| Test 2 | SNORA68 | UP |
| Test 2 | SNORA71A | UP |
| Test 2 | SNORA71B | UP |
| Test 2 | SNORA71C | UP |
| Test 2 | SNORA71D | UP |
| Test 2 | SNORA74B | UP |
| Test 2 | SNORA7B | UP |
| Test 2 | SNORA84 | UP |
| Test 2 | SNORA9 | UP |
| Test 2 | SNORD15B | UP |
| Test 2 | SNORD17 | UP |
| Test 2 | TM4SF19 | UP |
| Test 2 | TMEM179B | UP |
| Test 2 | TMEM45B | UP |
| Test 2 | TRMT6 | UP |
| Test 2 | UTP6 | UP |
| Test 2 | VSIG8 | UP |
| Test 2 | WDR60 | UP |
| Test 2 | WDR61 | UP |
| Test 2 | WFDC12 | UP |
| Test 2 | WIBG | UP |
| Test 2 | ARHGAP30 | DOWN |
| Test 2 | C17orf107 | DOWN |
| Test 2 | C22orf13 | DOWN |
| Test 2 | FAM100B | DOWN |
| Test 2 | FAM193B | DOWN |
| Test 2 | FAM210A | DOWN |
| Test 2 | FAM53C | DOWN |
| Test 2 | GPR108 | DOWN |
| Test 2 | GRAMD1A | DOWN |

TABLE 3-4

| | | |
|---|---|---|
| Test 2 | INO80D | DOWN |
| Test 2 | KIAA0232 | DOWN |
| Test 2 | MAP7D1 | DOWN |
| Test 2 | MLLT6 | DOWN |
| Test 2 | NCF1B | DOWN |
| Test 2 | PRR24 | DOWN |
| Test 2 | SDE2 | DOWN |
| Test 2 | SLED1 | DOWN |
| Test 2 | SMG1P1 | DOWN |
| Test 2 | TMEM167B | DOWN |

ii) RNA Expression Analysis—2

Data (read count values) on the expression level of RNA derived from the test subjects measured in the above section 2) was normalized by use of an approach called DESeq2. However, a sample in which 4161 or more genes were not detected was excluded, and only genes which produced expression level data without missing values in 90% or more sample test subjects in the expression level data on the test subjects in all the samples after exclusion were used in analysis given below. In the analysis, normalized count values obtained by use of an approach called DESeq2 were used.

Differentially expressed RNA which attained a corrected p value (FDR) of 0.25 or less in the likelihood ratio test in PD compared with the healthy subjects was identified on the basis of the SSL-derived RNA expression levels (normalized count values) of the healthy subjects and PD described above. In Test 1, the expression of 74 RNAs was increased in PD compared with the healthy subjects (Tables 4-1 and 4-2), and the expression of 209 RNAs was decreased therein (Tables 4-3 to 4-8). Meanwhile, in Test 2, the expression of 151 RNAs was increased (Tables 4-9 to 4-12), and the expression of 308 RNAs was decreased (Tables 4-13 to 4-20). The expression of 7 RNAs was increased in common between Test 1 and Test 2, and the expression of 10 RNAs was decreased in common therebetween (genes indicated by boldface in the tables).

TABLE 4-1

| Test | Symbol | Fold change | FDR | Regulation |
|---|---|---|---|---|
| Test 1 | ACOT2 | 2.120830751 | 0.171477109 | UP |
| Test 1 | ACOX3 | 1.905155929 | 0.192395571 | UP |
| Test 1 | ACTG1 | 0.591044977 | 0.216495961 | UP |
| Test 1 | AKT1S1 | 1.576633081 | 0.14231193 | UP |
| Test 1 | AMZ2 | 1.243802404 | 0.166431417 | UP |
| Test 1 | ANXA1 | 1.686938977 | 0.032012546 | UP |
| Test 1 | ANXA2 | 1.075474933 | 0.166431417 | UP |
| Test 1 | AQP3 | 2.056781943 | 0.207453699 | UP |
| Test 1 | AREG | 1.282649037 | 0.067390396 | UP |
| Test 1 | ARF5 | 0.81934585 | 0.218559941 | UP |
| Test 1 | ATP5E | 0.935310816 | 0.02664718 | UP |
| Test 1 | BCKDK | 1.060110294 | 0.057912524 | UP |
| Test 1 | BCR | 1.172433365 | 0.201130825 | UP |
| Test 1 | BSG | 1.119059971 | 0.247784004 | UP |
| Test 1 | C14orf2 | 0.678497763 | 0.213710325 | UP |
| Test 1 | CEBPA | 1.320916354 | 0.11590529 | UP |
| Test 1 | CHCHD2 | 1.191349912 | 0.004169691 | UP |
| Test 1 | CHMP5 | 1.072325081 | 0.069334816 | UP |
| Test 1 | COPE | 0.836423589 | 0.222937414 | UP |
| Test 1 | CORO1A | 1.434117261 | 0.142104577 | UP |
| Test 1 | CSDA | 0.738859106 | 0.242957593 | UP |
| Test 1 | DYNLT1 | 1.409545405 | 0.245101281 | UP |
| Test 1 | EIF4A3 | 1.316806302 | 0.102589353 | UP |
| Test 1 | EMP1 | 2.274143956 | 0.060301659 | UP |
| Test 1 | FLII | 1.575063373 | 0.212740547 | UP |
| Test 1 | GPR157 | 1.469120858 | 0.04751536 | UP |
| Test 1 | GPX3 | 1.455160573 | 0.081351393 | UP |
| Test 1 | HSPA1A | 1.401246844 | 0.128335102 | UP |
| Test 1 | KRT16 | 1.904813057 | 0.157035049 | UP |
| Test 1 | LOC100216546 | 1.822186187 | 0.192395571 | UP |
| Test 1 | LOC100288069 | 3.001066333 | 0.046401197 | UP |
| Test 1 | MESDC1 | 2.292885941 | 0.004532329 | UP |
| Test 1 | MIEN1 | 1.165319054 | 0.149707426 | UP |
| Test 1 | MKNK2 | 1.30619845 | 0.080683082 | UP |
| Test 1 | MNDA | 1.634027585 | 0.157035049 | UP |
| Test 1 | NEDD8 | 1.635919219 | 0.003678702 | UP |
| Test 1 | OTUD1 | 1.438403588 | 0.172366481 | UP |
| Test 1 | PIR | 1.736471923 | 0.187849008 | UP |
| Test 1 | PNISR | 1.817249957 | 0.166431417 | UP |
| Test 1 | POLR2J3 | 1.890184932 | 0.140445468 | UP |

TABLE 4-2

| | | | | |
|---|---|---|---|---|
| Test 1 | POLR2L | 1.140600646 | 0.205453026 | UP |
| Test 1 | PQLC1 | 1.211887627 | 0.137092675 | UP |
| Test 1 | PRELID1 | 0.985264241 | 0.185048528 | UP |
| Test 1 | PRKAA1 | 1.063955765 | 0.247784004 | UP |
| Test 1 | PSMA7 | 0.928612269 | 0.191314244 | UP |
| Test 1 | PSMD4 | 1.210300048 | 0.016937899 | UP |
| Test 1 | PTGS2 | 1.716429174 | 0.165136091 | UP |
| Test 1 | RASAL1 | 1.378706419 | 0.240134481 | UP |
| Test 1 | RNASET2 | 1.690750811 | 0.221565223 | UP |
| Test 1 | RNF217 | 1.665796573 | 0.180188714 | UP |
| Test 1 | RPL13 | 1.656906532 | 0.004532329 | UP |
| Test 1 | S100A8 | 1.385197789 | 0.211636176 | UP |
| Test 1 | SDC4 | 1.764459258 | 0.186471701 | UP |
| Test 1 | SERPINB4 | 2.405038672 | 0.093218948 | UP |
| Test 1 | SLC25A3 | 0.957786481 | 0.097706633 | UP |
| Test 1 | SLPI | 1.223952779 | 0.240134481 | UP |
| Test 1 | SNORA24 | 1.41317214 | 0.022725658 | UP |
| Test 1 | SNORA50 | 2.364388841 | 0.035336192 | UP |
| Test 1 | SNORA57 | 2.930672887 | 0.004532329 | UP |
| Test 1 | SNORA8 | 1.396949079 | 0.142963515 | UP |
| Test 1 | SNORA9 | 1.523014565 | 0.142763401 | UP |
| Test 1 | SOCS3 | 1.228282723 | 0.240134481 | UP |
| Test 1 | TIMP1 | 1.378822071 | 0.165136091 | UP |
| Test 1 | TMCC3 | 1.173598961 | 0.209941538 | UP |
| Test 1 | TRMT44 | 1.881661647 | 0.11590529 | UP |
| Test 1 | TSPO | 1.290086722 | 0.008973844 | UP |
| Test 1 | TUBA1C | 1.180331715 | 0.067390396 | UP |
| Test 1 | UQCRB | 0.953505252 | 0.166431417 | UP |
| Test 1 | UQCRC1 | 1.171312279 | 0.032012546 | UP |
| Test 1 | UQCRFS1 | 1.118741793 | 0.157035049 | UP |
| Test 1 | VEGFA | 1.170135516 | 0.14231193 | UP |
| Test 1 | ZFP36L2 | 1.597906298 | 0.212740547 | UP |
| Test 1 | ZNF410 | 1.411824416 | 0.017419551 | UP |
| Test 1 | ZSWIM6 | 1.164512926 | 0.200368845 | UP |

TABLE 4-3

| | | | | |
|---|---|---|---|---|
| Test 1 | AATF | −1.772740768 | 0.028713164 | DOWN |
| Test 1 | ADRBK2 | −1.565594145 | 0.214520377 | DOWN |
| Test 1 | AHSA1 | −1.855551649 | 0.04751536 | DOWN |
| Test 1 | AIDA | −1.498618103 | 0.137671023 | DOWN |
| Test 1 | ANKRD12 | −3.218993782 | 0.000308536 | DOWN |
| Test 1 | ANXA3 | −2.160717204 | 0.198639769 | DOWN |
| Test 1 | AP3B1 | −2.069780342 | 0.01186505 | DOWN |
| Test 1 | APH1A | −1.601000274 | 0.044757805 | DOWN |
| Test 1 | API5 | −2.46515534 | 0.022303042 | DOWN |
| Test 1 | APLP2 | −1.302316687 | 0.209941538 | DOWN |
| Test 1 | ARID4B | −2.568474682 | 0.013052784 | DOWN |
| Test 1 | ARPC1A | −1.745179478 | 0.179294587 | DOWN |
| Test 1 | ARPC3 | −1.326436202 | 0.04751536 | DOWN |
| Test 1 | ATG12 | −1.485488983 | 0.201316711 | DOWN |
| Test 1 | ATP2A2 | −1.658130821 | 0.11590529 | DOWN |
| Test 1 | ATP5J2 | −0.961630702 | 0.153747759 | DOWN |
| Test 1 | ATP6AP2 | −1.690782229 | 0.028713164 | DOWN |
| Test 1 | ATP6V0C | −0.92893591 | 0.142104577 | DOWN |
| Test 1 | ATP6V1G1 | −0.835824694 | 0.17928974 | DOWN |
| Test 1 | BAG1 | −1.308033408 | 0.154290596 | DOWN |
| Test 1 | BHLHE40 | −1.574553746 | 0.003238712 | DOWN |
| Test 1 | BTF3 | −0.962144231 | 0.166431417 | DOWN |
| Test 1 | BTG1 | −1.205115472 | 0.069804405 | DOWN |
| Test 1 | BUD31 | −1.680224231 | 0.140744395 | DOWN |
| Test 1 | C14orf178 | −1.827426054 | 0.079281961 | DOWN |
| Test 1 | CAPZA1 | −1.035082105 | 0.212749025 | DOWN |
| Test 1 | CAPZA2 | −2.593953142 | 0.000573479 | DOWN |
| Test 1 | CBFB | −1.809532507 | 0.149707426 | DOWN |
| Test 1 | CCDC93 | −2.428995887 | 0.027093818 | DOWN |
| Test 1 | CCL3 | −2.617993487 | 0.022303042 | DOWN |
| Test 1 | CCNI | −2.705241728 | 8.8856E−05 | DOWN |
| Test 1 | CDC42 | −1.694231647 | 0.000238125 | DOWN |
| Test 1 | CHMP2A | −1.807256465 | 7.81525E−05 | DOWN |
| Test 1 | CHMP2B | −1.356411536 | 0.044486644 | DOWN |
| Test 1 | CHMP3 | −1.308675973 | 0.11590529 | DOWN |
| Test 1 | CIRBP | −1.490579305 | 0.028713164 | DOWN |
| Test 1 | CLIC4 | −2.178997823 | 0.004532329 | DOWN |
| Test 1 | CLIP1 | −1.655621373 | 0.157035049 | DOWN |
| Test 1 | CLK1 | −1.575308686 | 0.238174085 | DOWN |
| Test 1 | CLNS1A | −2.441026451 | 0.067390396 | DOWN |
| Test 1 | CNBP | −1.490648544 | 0.00052874 | DOWN |

TABLE 4-4

| | | | | |
|---|---|---|---|---|
| Test 1 | COPB2 | −1.508519088 | 0.201672143 | DOWN |
| Test 1 | CPA4 | −2.565357457 | 0.06078135 | DOWN |
| Test 1 | CPM | −3.185069888 | 0.004169691 | DOWN |
| Test 1 | CS | −1.412980886 | 0.155875067 | DOWN |
| Test 1 | CSF1 | −2.232962038 | 0.089756063 | DOWN |
| Test 1 | CXCR4 | −1.852473527 | 0.024085385 | DOWN |
| Test 1 | CYBB | −1.547680875 | 0.131595057 | DOWN |
| Test 1 | DCUN1D1 | −3.29687005 | 0.003016508 | DOWN |
| Test 1 | DDX21 | −1.953597404 | 0.17928974 | DOWN |
| Test 1 | DDX5 | −0.951202357 | 0.157035049 | DOWN |
| Test 1 | DICER1 | −2.438540315 | 0.053386182 | DOWN |
| Test 1 | DLD | −2.382342017 | 0.104358929 | DOWN |
| Test 1 | DNAJC15 | −1.735325528 | 0.212749025 | DOWN |
| Test 1 | DNAJC3 | −1.80993238 | 0.007533471 | DOWN |
| Test 1 | DR1 | −2.223672473 | 0.006574907 | DOWN |
| Test 1 | EEE1B2 | −1.013792824 | 0.153777482 | DOWN |
| Test 1 | EGR2 | −0.988003468 | 0.166431417 | DOWN |
| Test 1 | EIF2S2 | −1.424601763 | 0.192395571 | DOWN |
| Test 1 | EIF5A | −0.627832441 | 0.209941538 | DOWN |
| Test 1 | ELF1 | −1.71759088 | 0.02334994 | DOWN |
| Test 1 | EML4 | −3.139954556 | 0.000956919 | DOWN |
| Test 1 | EP300 | −2.931533156 | 0.003577618 | DOWN |
| Test 1 | EPS15 | −1.351127393 | 0.110751825 | DOWN |
| Test 1 | ERBB2IP | −0.983202956 | 0.157035049 | DOWN |
| Test 1 | ETF1 | −2.099561074 | 0.000858271 | DOWN |
| Test 1 | ETV6 | −1.217282765 | 0.161738652 | DOWN |
| Test 1 | EVL | −2.13743363 | 0.201316711 | DOWN |
| Test 1 | EZR | −1.337706169 | 0.104520242 | DOWN |
| Test 1 | FAM100A | −1.441515396 | 0.231685231 | DOWN |
| Test 1 | FAM126A | −3.771877733 | 0.026955947 | DOWN |
| Test 1 | FAM160A1 | −1.673651499 | 0.043287134 | DOWN |
| Test 1 | FNTA | −4.395569996 | 0.032461153 | DOWN |
| Test 1 | FUBP1 | −3.812291612 | 0.000308536 | DOWN |
| Test 1 | FYTTD1 | −1.74478099 | 0.186471701 | DOWN |
| Test 1 | G3BP2 | −1.313544933 | 0.187849008 | DOWN |
| Test 1 | GABARAP | −1.10082081 | 0.156429231 | DOWN |
| Test 1 | GABARAPL1 | −1.322693883 | 0.060301659 | DOWN |
| Test 1 | GLTP | −2.377100594 | 0.11590529 | DOWN |
| Test 1 | GLTSCR2 | −1.989132848 | 0.004532329 | DOWN |
| Test 1 | GOLGA8B | −1.533924827 | 0.213256993 | DOWN |
| Test 1 | GRB2 | −1.22550846 | 0.154290596 | DOWN |

TABLE 4-5

| | | | | |
|---|---|---|---|---|
| Test 1 | HBP1 | −1.691481895 | 0.02450771 | DOWN |
| Test 1 | HELZ | −2.689866366 | 0.003678702 | DOWN |
| Test 1 | HIF1A | −1.224827769 | 0.238174085 | DOWN |
| Test 1 | HINT1 | −1.453762692 | 0.04751536 | DOWN |
| Test 1 | HINT3 | −1.947152032 | 0.140445468 | DOWN |
| Test 1 | HIST1H1E | −1.670140606 | 0.103600407 | DOWN |
| Test 1 | HMGN1 | −2.063165682 | 0.073126006 | DOWN |
| Test 1 | HNRNPA2B1 | −1.274269915 | 0.142104577 | DOWN |
| Test 1 | HNRNPK | −1.96640437 | 0.000238125 | DOWN |
| Test 1 | HNRNPU | −1.703715606 | 0.009237092 | DOWN |
| Test 1 | IARS2 | −2.502578081 | 0.04751536 | DOWN |
| Test 1 | ICAM1 | −2.383130311 | 0.162465282 | DOWN |
| Test 1 | IDE | −1.862223274 | 0.11590529 | DOWN |
| Test 1 | IER3IP1 | −2.129040887 | 0.191902648 | DOWN |
| Test 1 | JAK1 | −2.478429677 | 0.04751536 | DOWN |
| Test 1 | JMY | −2.263496873 | 0.155218477 | DOWN |
| Test 1 | KAT2B | −1.550256904 | 0.157035049 | DOWN |
| Test 1 | KIAA1551 | −1.367628259 | 0.228182221 | DOWN |
| Test 1 | KIF16B | −1.712088316 | 0.170079315 | DOWN |
| Test 1 | KLF10 | −2.507920855 | 0.01186505 | DOWN |
| Test 1 | KLF3 | −2.671224065 | 0.011682374 | DOWN |
| Test 1 | LGALSL | −1.868246009 | 0.165136091 | DOWN |
| Test 1 | MARCH7 | −1.358142867 | 0.242073043 | DOWN |
| Test 1 | MBD2 | −1.966385172 | 0.008794332 | DOWN |
| Test 1 | MBD6 | −2.243104033 | 0.157035049 | DOWN |
| Test 1 | MDM2 | −2.174980192 | 0.067390396 | DOWN |
| Test 1 | MED13L | −1.63902893 | 0.104520242 | DOWN |
| Test 1 | MED19 | −3.581005956 | 0.007535427 | DOWN |
| Test 1 | MRPL15 | −2.386765875 | 0.094888262 | DOWN |
| Test 1 | NAPA | −1.420133442 | 0.067390396 | DOWN |
| Test 1 | NR4A2 | −1.256772697 | 0.231685231 | DOWN |
| Test 1 | NRBF2 | −0.871916325 | 0.124019241 | DOWN |
| Test 1 | NRBP1 | −1.122530881 | 0.242957593 | DOWN |
| Test 1 | NSFP1 | −1.167024718 | 0.104520242 | DOWN |
| Test 1 | OGFRL1 | −1.459613162 | 0.06499322 | DOWN |
| Test 1 | P4HB | −0.938800796 | 0.184113444 | DOWN |
| Test 1 | PAIP2 | −1.484416116 | 0.04751536 | DOWN |
| Test 1 | PDXK | −1.265283201 | 0.246917684 | DOWN |
| Test 1 | PGK1 | −0.921105178 | 0.135858235 | DOWN |
| Test 1 | PGRMC2 | −2.309010058 | 0.142104577 | DOWN |
| Test 1 | PHF20L1 | −2.098809369 | 0.18841032 | DOWN |

TABLE 4-6

| | | | | |
|---|---|---|---|---|
| Test 1 | PHF5A | −2.478924839 | 0.04751536 | DOWN |
| Test 1 | PIKFYVE | −2.246740668 | 0.247784004 | DOWN |
| Test 1 | PLA2G7 | −1.92588687 | 0.126138663 | DOWN |
| Test 1 | POLR2A | −1.007640822 | 0.245101281 | DOWN |
| Test 1 | PTPN12 | −2.409768904 | 0.003238712 | DOWN |
| Test 1 | QARS | −2.502319653 | 0.004169691 | DOWN |
| Test 1 | RAB14 | −2.991933128 | 0.001713645 | DOWN |
| Test 1 | RAB9A | −1.966991656 | 0.214127581 | DOWN |
| Test 1 | RABGEF1 | −2.346307336 | 0.004532329 | DOWN |
| Test 1 | RAP1A | −1.627957688 | 0.006574907 | DOWN |
| Test 1 | RAP1B | −0.938277209 | 0.128335102 | DOWN |
| Test 1 | RHOA | −0.846384811 | 0.166431417 | DOWN |
| Test 1 | RIOK3 | −1.537528915 | 0.201316711 | DOWN |
| Test 1 | RMND5A | −1.727209574 | 0.104520242 | DOWN |
| Test 1 | RNASEK | −0.803995199 | 0.134092229 | DOWN |
| Test 1 | RPL10 | −2.075102838 | 0.002320011 | DOWN |
| Test 1 | RPL13AP20 | −0.833469251 | 0.173586614 | DOWN |
| Test 1 | RPL15 | −1.780679733 | 0.00823501 | DOWN |
| Test 1 | RPL19 | −0.964036328 | 0.174007936 | DOWN |
| Test 1 | RPL24 | −1.300785402 | 0.131657412 | DOWN |
| Test 1 | RPL26 | −1.353489893 | 0.079440025 | DOWN |
| Test 1 | RPL28 | −0.831266984 | 0.215698645 | DOWN |
| Test 1 | RPL36AL | −1.835109641 | 0.005258055 | DOWN |
| Test 1 | RPL5 | −1.795729934 | 0.021525976 | DOWN |
| Test 1 | RPL6 | −2.49204435 | 0.003942597 | DOWN |
| Test 1 | RPS20 | −1.754918062 | 0.004169691 | DOWN |
| Test 1 | RPS25 | −1.28675117 | 0.01535602 | DOWN |
| Test 1 | RPS9 | −0.961456424 | 0.053716153 | DOWN |
| Test 1 | S100A10 | −1.295076872 | 0.166431417 | DOWN |
| Test 1 | S100A11 | −1.030462079 | 0.154290596 | DOWN |
| Test 1 | SCAF11 | −1.77369601 | 0.11590529 | DOWN |
| Test 1 | SCYL2 | −1.943402128 | 0.013505503 | DOWN |
| Test 1 | SDF4 | −2.085196384 | 0.170106979 | DOWN |
| Test 1 | SEC11C | −2.792373921 | 0.008933234 | DOWN |
| Test 1 | SEC24A | −3.07035687 | 0.038419833 | DOWN |
| Test 1 | SEPT11 | −2.357033916 | 0.153777482 | DOWN |
| Test 1 | SEPT2 | −1.74873368 | 0.004680722 | DOWN |
| Test 1 | SERINC1 | −1.248273951 | 0.073126006 | DOWN |
| Test 1 | SERINC3 | −1.030053705 | 0.025949689 | DOWN |
| Test 1 | SERPINA12 | −3.986279444 | 0.033389704 | DOWN |
| Test 1 | SERPINB9 | −1.250923726 | 0.209941538 | DOWN |

TABLE 4-7

| | | | | |
|---|---|---|---|---|
| Test 1 | SERTAD2 | −2.029830502 | 0.192395571 | DOWN |
| Test 1 | SET | −1.937444712 | 0.007533471 | DOWN |
| Test 1 | SH3BGRL3 | −0.663305735 | 0.067390396 | DOWN |
| Test 1 | SLMO2 | −1.81329058 | 0.137966894 | DOWN |
| Test 1 | SMS | −2.704517802 | 0.045626018 | DOWN |
| Test 1 | SNAP29 | −1.590085581 | 0.16114262 | DOWN |
| Test 1 | SNORA53 | −1.586512685 | 0.15598759 | DOWN |
| Test 1 | SNX13 | −3.004474961 | 0.000999599 | DOWN |
| Test 1 | SNX9 | −1.779958051 | 0.028713164 | DOWN |
| Test 1 | SREK1IP1 | −1.839855519 | 0.154290596 | DOWN |
| Test 1 | SRSF5 | −0.984113666 | 0.13064644 | DOWN |
| Test 1 | SSR2 | −1.749337253 | 0.146923279 | DOWN |
| Test 1 | SSU72 | −1.245325192 | 0.027093818 | DOWN |
| Test 1 | STK24 | −2.734918584 | 0.000596053 | DOWN |
| Test 1 | STT3B | −1.951800228 | 0.150544954 | DOWN |
| Test 1 | TAF10 | −1.32452647 | 0.094888262 | DOWN |
| Test 1 | TAOK1 | −1.927349024 | 0.030576015 | DOWN |
| Test 1 | TERF2IP | −1.792863603 | 0.084366841 | DOWN |
| Test 1 | TLK2 | −2.605906707 | 0.170106979 | DOWN |
| Test 1 | TMA7 | −1.367895195 | 0.028713164 | DOWN |
| Test 1 | TMEM106B | −1.86835998 | 0.247784004 | DOWN |
| Test 1 | TMEM127 | −1.190703794 | 0.044486644 | DOWN |
| Test 1 | TMEM167B | −1.796713966 | 0.116792089 | DOWN |
| Test 1 | TNFSF13B | −1.78814654 | 0.131657412 | DOWN |
| Test 1 | TPGS2 | −1.809874669 | 0.11590529 | DOWN |
| Test 1 | TRAM1 | −1.839546005 | 0.092035865 | DOWN |
| Test 1 | TRIP12 | −1.725435313 | 0.025949689 | DOWN |
| Test 1 | TRPM7 | −2.038357771 | 0.182595713 | DOWN |
| Test 1 | TSG101 | −1.005237989 | 0.209643258 | DOWN |
| Test 1 | TXNL1 | −1.549871273 | 0.032012546 | DOWN |
| Test 1 | UBE2A | −1.592829916 | 0.088783965 | DOWN |
| Test 1 | UBE2B | −1.436513364 | 0.078520181 | DOWN |
| Test 1 | UBE2H | −3.405818637 | 0.004532329 | DOWN |
| Test 1 | USMG5 | −1.046390149 | 0.136226208 | DOWN |
| Test 1 | USP22 | −1.181507483 | 0.174760541 | DOWN |
| Test 1 | USP53 | −3.761488613 | 0.006574907 | DOWN |
| Test 1 | USP6NL | −1.79126036 | 0.192642777 | DOWN |
| Test 1 | USP7 | −1.993708629 | 0.079281961 | DOWN |
| Test 1 | WIPF1 | −2.742465049 | 0.000134039 | DOWN |
| Test 1 | WTAP | −1.446170337 | 0.200942058 | DOWN |
| Test 1 | XBP1 | −1.326123865 | 0.14231193 | DOWN |

TABLE 4-8

| | | | | |
|---|---|---|---|---|
| Test 1 | YWHAQ | −3.230153729 | 0.000308536 | DOWN |
| Test 1 | ZCRB1 | −2.455139576 | 0.104520242 | DOWN |
| Test 1 | ZMAT2 | −1.635539488 | 0.104520242 | DOWN |
| Test 1 | ZNF148 | −2.237573981 | 0.088783965 | DOWN |

TABLE 4-9

| | | | | |
|---|---|---|---|---|
| Test 2 | ALOX12B | 0.723735806 | 0.167674326 | UP |
| Test 2 | ANXA1 | 0.789867752 | 0.014394956 | UP |
| Test 2 | AQP3 | 0.599212307 | 0.197195688 | UP |
| Test 2 | ATP12A | 0.431246438 | 0.191525939 | UP |
| Test 2 | ATP5B | 0.209786056 | 0.203917134 | UP |
| Test 2 | ATP5I | 0.542925568 | 0.018324394 | UP |
| Test 2 | ATP5O | 0.365101203 | 0.108586621 | UP |
| Test 2 | BAG3 | 0.698056003 | 0.041402036 | UP |
| Test 2 | C6orf132 | 0.427761744 | 0.221553842 | UP |
| Test 2 | CALM1 | 0.261573948 | 0.180087728 | UP |
| Test 2 | CASP14 | 0.575805082 | 0.189823772 | UP |
| Test 2 | CAST | 0.391443433 | 0.073143922 | UP |
| Test 2 | CDSN | 0.418002881 | 0.233357246 | UP |
| Test 2 | CLIC3 | 1.046049107 | 0.032990086 | UP |
| Test 2 | CNFN | 0.840234841 | 0.003993974 | UP |
| Test 2 | COX4I1 | 0.246033363 | 0.114612949 | UP |
| Test 2 | COX8A | 0.239739307 | 0.185751097 | UP |
| Test 2 | CRABP2 | 0.875558417 | 0.002325233 | UP |
| Test 2 | CST6 | 0.405606922 | 0.230255192 | UP |
| Test 2 | CTSC | 0.543171172 | 0.248489765 | UP |
| Test 2 | DNAJA1 | 0.280489771 | 0.204723138 | UP |
| Test 2 | DYNLL1 | 0.264998709 | 0.248133155 | UP |
| Test 2 | EEF1B2 | 0.323291572 | 0.113394678 | UP |
| Test 2 | EIF1AX | 0.484502482 | 0.057323067 | UP |
| Test 2 | EIF3K | 0.451680409 | 0.021385054 | UP |
| Test 2 | ELF3 | 0.673774244 | 0.148695977 | UP |
| Test 2 | EMP1 | 1.53672252 | 0.000620279 | UP |
| Test 2 | EPHX3 | 0.986214766 | 0.023042585 | UP |
| Test 2 | FABP9 | 1.266189045 | 0.001096741 | UP |
| Test 2 | GNB2L1 | 0.271603302 | 0.203917134 | UP |
| Test 2 | GRHL3 | 0.482949482 | 0.207332199 | UP |
| Test 2 | HIST1H4E | 0.685060675 | 0.03912317 | UP |
| Test 2 | HIST1H4H | 0.770040391 | 0.004197223 | UP |
| Test 2 | HMGCS1 | 0.365100839 | 0.246563533 | UP |
| Test 2 | HMOX2 | 0.417111371 | 0.079852349 | UP |
| Test 2 | HSP90AA1 | 0.33731324 | 0.228358936 | UP |
| Test 2 | HSPB1 | 0.370513163 | 0.20793407 | UP |
| Test 2 | IVL | 1.089468005 | 0.001079959 | UP |
| Test 2 | KLF5 | 0.637821501 | 0.203917134 | UP |
| Test 2 | KLK13 | 0.693077306 | 0.09306963 | UP |
| Test 2 | KLK7 | 0.637766385 | 0.102583472 | UP |

TABLE 4-10

| | | | | |
|---|---|---|---|---|
| Test 2 | KRT10 | 0.786915063 | 0.214896999 | UP |
| Test 2 | KRT14 | 0.556501136 | 0.094970832 | UP |
| Test 2 | KRT16 | 0.398735989 | 0.203917134 | UP |
| Test 2 | KRT25 | 1.240192706 | 0.001079959 | UP |
| Test 2 | KRT27 | 1.042441735 | 0.007811119 | UP |
| Test 2 | KRT5 | 1.059130956 | 0.035341619 | UP |
| Test 2 | KRT6A | 0.539579298 | 0.094970832 | UP |
| Test 2 | KRT71 | 1.005044058 | 0.009445737 | UP |
| Test 2 | KRT72 | 0.953782057 | 0.019401367 | UP |
| Test 2 | KRT74 | 0.942811431 | 0.110496509 | UP |
| Test 2 | KRTAP5-3 | 0.864276264 | 0.240103288 | UP |
| Test 2 | KRTDAP | 0.532072112 | 0.094970832 | UP |
| Test 2 | LCE2C | 0.468549536 | 0.192091234 | UP |
| Test 2 | LCE2D | 0.445193874 | 0.230255192 | UP |
| Test 2 | LCE3D | 0.583545316 | 0.093863146 | UP |
| Test 2 | LCE3E | 0.589208528 | 0.087587726 | UP |
| Test 2 | LCN2 | 0.716728817 | 0.032990086 | UP |
| Test 2 | LNX1 | 0.777864317 | 0.019217132 | UP |
| Test 2 | LRRC15 | 0.63618229 | 0.127005018 | UP |
| Test 2 | NDRG2 | 0.34352027 | 0.204723138 | UP |
| Test 2 | NDUFA4L2 | 0.939272871 | 0.02821497 | UP |
| Test 2 | NDUFB11 | 0.372680705 | 0.176688503 | UP |
| Test 2 | NDUFB2 | 0.621006658 | 0.079217394 | UP |
| Test 2 | NDUFB8 | 0.382724687 | 0.137176161 | UP |
| Test 2 | NDUFS5 | 0.475805215 | 0.070897185 | UP |
| Test 2 | NSFL1C | 0.305386997 | 0.225295536 | UP |
| Test 2 | NUMA1 | 0.361671318 | 0.101888953 | UP |
| Test 2 | PDZK1IP1 | 0.714851787 | 0.203917134 | UP |
| Test 2 | PINLYP | 0.705842244 | 0.157749295 | UP |
| Test 2 | PKP1 | 0.464155403 | 0.180898511 | UP |
| Test 2 | PNP | 0.323977868 | 0.203917134 | UP |
| Test 2 | POLR2L | 0.388253793 | 0.070687016 | UP |
| Test 2 | PPL | 1.076945835 | 0.035341619 | UP |
| Test 2 | PPP2R2A | 0.337886437 | 0.236569609 | UP |
| Test 2 | PRR9 | 0.834659426 | 0.019217132 | UP |
| Test 2 | PRSS3 | 0.616978345 | 0.094970832 | UP |
| Test 2 | PSMC2 | 0.289510565 | 0.240103288 | UP |
| Test 2 | RBBP4 | 0.438634443 | 0.203917134 | UP |
| Test 2 | RMRP | 0.530656521 | 0.034346406 | UP |
| Test 2 | ROMO1 | 0.278856205 | 0.213337813 | UP |
| Test 2 | RPL10A | 0.267591219 | 0.19331656 | UP |

TABLE 4-11

| | | | | |
|---|---|---|---|---|
| Test 2 | RPL11 | 0.272049357 | 0.188973375 | UP |
| Test 2 | RPL12 | 0.272231447 | 0.203917134 | UP |
| Test 2 | RPL13A | 0.332882836 | 0.087496195 | UP |
| Test 2 | RPL18 | 0.23631901 | 0.203917134 | UP |
| Test 2 | RPL21 | 0.234057222 | 0.228358936 | UP |
| Test 2 | RPL26 | 0.275178488 | 0.203917134 | UP |
| Test 2 | RPL27 | 0.25361932 | 0.203917134 | UP |
| Test 2 | RPL27A | 0.286064033 | 0.203917134 | UP |
| Test 2 | RPL29 | 0.227323201 | 0.202573111 | UP |
| Test 2 | RPL3 | 0.267937405 | 0.185751097 | UP |
| Test 2 | RPL30 | 0.22303117 | 0.208405568 | UP |
| Test 2 | RPL32 | 0.366698061 | 0.05120094 | UP |
| Test 2 | RPL35 | 0.353161175 | 0.075426886 | UP |
| Test 2 | RPL36 | 0.321842862 | 0.084037369 | UP |
| Test 2 | RPL36A | 0.338476857 | 0.089104057 | UP |
| Test 2 | RPL37A | 0.429627149 | 0.035341619 | UP |
| Test 2 | RPL38 | 0.361505145 | 0.069516286 | UP |
| Test 2 | RPL7 | 0.407722145 | 0.013807457 | UP |
| Test 2 | RPL7A | 0.3865274 | 0.033308231 | UP |
| Test 2 | RPLP0 | 0.475836697 | 0.00985997 | UP |
| Test 2 | RPLP1 | 0.466227457 | 0.019217132 | UP |
| Test 2 | RPLP2 | 0.351641372 | 0.151691535 | UP |
| Test 2 | RPS10 | 0.278100919 | 0.129200547 | UP |
| Test 2 | RPS12 | 0.557211765 | 0.001079959 | UP |
| Test 2 | RPS15 | 0.343773569 | 0.061406696 | UP |
| Test 2 | RPS15A | 0.241359539 | 0.204723138 | UP |
| Test 2 | RPS18 | 0.545456358 | 0.003707257 | UP |
| Test 2 | RPS19 | 0.304856429 | 0.188426153 | UP |
| Test 2 | RPS21 | 0.299295052 | 0.230255192 | UP |
| Test 2 | RPS26 | 0.495375545 | 0.056702789 | UP |
| Test 2 | RPS28 | 0.346942763 | 0.045753193 | UP |
| Test 2 | RPS3 | 0.467901737 | 0.121272078 | UP |
| Test 2 | RPS4X | 0.403982139 | 0.053047353 | UP |
| Test 2 | RPS5 | 0.360449408 | 0.079217394 | UP |
| Test 2 | RPS6 | 0.323863058 | 0.083321514 | UP |
| Test 2 | RPS8 | 0.276154805 | 0.156533343 | UP |
| Test 2 | S100A14 | 0.77672344 | 0.035341619 | UP |
| Test 2 | S100A7 | 0.49351568 | 0.203917134 | UP |
| Test 2 | S100A7A | 0.82107465 | 0.057323067 | UP |
| Test 2 | S100A9 | 0.415203898 | 0.204723138 | UP |
| Test 2 | SBDS | 0.433629133 | 0.094970832 | UP |

TABLE 4-12

| | | | | |
|---|---|---|---|---|
| Test 2 | SBSN | 0.400950246 | 0.248266371 | UP |
| Test 2 | SERPINB4 | 0.673429357 | 0.142882428 | UP |
| Test 2 | SERPINB5 | 0.437096337 | 0.185215103 | UP |
| Test 2 | SFN | 1.052956601 | 0.013115227 | UP |
| Test 2 | SLURP1 | 0.772094195 | 0.246563533 | UP |
| Test 2 | SNORA16A | 0.61798567 | 0.035341619 | UP |
| Test 2 | SNORA24 | 0.379856346 | 0.249405298 | UP |
| Test 2 | SNORA52 | 0.527159643 | 0.109351493 | UP |
| Test 2 | SNORA63 | 0.384997588 | 0.248489765 | UP |
| Test 2 | SNORA68 | 0.601608716 | 0.045753193 | UP |
| Test 2 | SNORA71A | 0.545397641 | 0.114065157 | UP |
| Test 2 | SNORD15B | 0.48371262 | 0.126316838 | UP |
| Test 2 | SPRR1A | 0.428636075 | 0.19331656 | UP |
| Test 2 | SPRR1B | 0.495978239 | 0.101888953 | UP |
| Test 2 | SPRR2D | 0.974665073 | 0.001096741 | UP |
| Test 2 | SPRR2E | 0.723183713 | 0.019217132 | UP |
| Test 2 | SPRR2F | 0.886789503 | 0.029464953 | UP |
| Test 2 | TCHH | 1.050322557 | 0.004197223 | UP |
| Test 2 | TCHHL1 | 1.1133749 | 0.019217132 | UP |
| Test 2 | TMOD3 | 0.466106975 | 0.180087728 | UP |
| Test 2 | TMPRSS11E | 0.464901468 | 0.240103288 | UP |
| Test 2 | UBE2L3 | 0.495979301 | 0.003707257 | UP |
| Test 2 | UBL3 | 0.384648798 | 0.129200547 | UP |
| Test 2 | UQCR11 | 0.316274231 | 0.127005018 | UP |
| Test 2 | UQCRH | 0.330479444 | 0.156533343 | UP |
| Test 2 | UXT | 0.285545238 | 0.240103288 | UP |
| Test 2 | WWC1 | 0.509668226 | 0.241275153 | UP |
| Test 2 | WWTR1 | 0.5963163 | 0.101888953 | UP |

TABLE 4-13

| | | | | |
|---|---|---|---|---|
| Test 2 | A2M | −0.718075308 | 0.148123279 | DOWN |
| Test 2 | AADACL3 | −0.58202357 | 0.213337813 | DOWN |
| Test 2 | ABHD5 | −0.384116317 | 0.248266371 | DOWN |
| Test 2 | ABTB1 | −0.48310785 | 0.207332199 | DOWN |
| Test 2 | ACSL5 | −0.962971251 | 0.127005018 | DOWN |
| Test 2 | ADAM8 | −0.628191368 | 0.184292206 | DOWN |
| Test 2 | ADORA2A | −1.177098212 | 0.018559854 | DOWN |
| Test 2 | AGTRAP | −0.841095761 | 0.10645836 | DOWN |
| Test 2 | AKR7A2 | −0.599763384 | 0.178842249 | DOWN |
| Test 2 | ALPL | −0.832944054 | 0.240103288 | DOWN |
| Test 2 | AMPD2 | −0.618352376 | 0.240103288 | DOWN |
| Test 2 | ANKRD22 | −0.356210317 | 0.240103288 | DOWN |
| Test 2 | AP5B1 | −0.586537694 | 0.213337813 | DOWN |
| Test 2 | ARF1 | −0.211680435 | 0.097540633 | DOWN |
| Test 2 | ARF5 | −0.266734474 | 0.203917134 | DOWN |
| Test 2 | ARHGAP1 | −0.422844029 | 0.143273824 | DOWN |
| Test 2 | ARHGAP30 | −0.800282483 | 0.083321514 | DOWN |
| Test 2 | ARHGEF2 | −0.553024402 | 0.203917134 | DOWN |
| Test 2 | ARID3A | −0.972422942 | 0.122046617 | DOWN |
| Test 2 | ARL5B | −0.502970468 | 0.203976387 | DOWN |
| Test 2 | ARRB2 | −0.503495008 | 0.156533343 | DOWN |
| Test 2 | ASAHI | −0.573971415 | 0.228358936 | DOWN |
| Test 2 | ATG2A | −0.506283174 | 0.036440805 | DOWN |
| Test 2 | ATHL1 | −1.056342135 | 0.050301518 | DOWN |
| Test 2 | ATP6V0C | −0.454978704 | 0.029798738 | DOWN |
| Test 2 | BASP1 | −0.555927115 | 0.185751097 | DOWN |
| Test 2 | BCKDK | −0.31920705 | 0.203917134 | DOWN |
| Test 2 | BCL2L1 | −0.328248615 | 0.235721155 | DOWN |
| Test 2 | BHLHE40 | −0.401373324 | 0.189293656 | DOWN |
| Test 2 | BRD4 | −0.592405451 | 0.185751097 | DOWN |
| Test 2 | C17orf107 | −0.743835661 | 0.213337813 | DOWN |
| Test 2 | C1orf43 | −0.386791488 | 0.034346406 | DOWN |
| Test 2 | C22orf13 | −0.651369541 | 0.073352416 | DOWN |
| Test 2 | C2CD2 | −0.578876548 | 0.248266371 | DOWN |
| Test 2 | C6orf106 | −0.657660723 | 0.019217132 | DOWN |
| Test 2 | CANT1 | −0.715521843 | 0.191525939 | DOWN |
| Test 2 | CCDC86 | −0.492588982 | 0.240103288 | DOWN |
| Test 2 | CCL3 | −1.013989016 | 0.019217132 | DOWN |
| Test 2 | CCL3L3 | −1.008726691 | 0.019217132 | DOWN |
| Test 2 | CCL4 | −0.756395613 | 0.087496195 | DOWN |
| Test 2 | CCNI | −0.297462333 | 0.191525939 | DOWN |

TABLE 4-14

| | | | | |
|---|---|---|---|---|
| Test 2 | CCNY | −0.412042019 | 0.127005018 | DOWN |
| Test 2 | CCRL2 | −0.793390941 | 0.12802663 | DOWN |
| Test 2 | CCSAP | −0.460516843 | 0.248489765 | DOWN |
| Test 2 | CD300A | −0.684472303 | 0.203917134 | DOWN |
| Test 2 | CD36 | −0.533682671 | 0.204049804 | DOWN |
| Test 2 | CD63 | −0.379385495 | 0.194315694 | DOWN |
| Test 2 | CD82 | −0.716610607 | 0.090458126 | DOWN |
| Test 2 | CD83 | −0.481612361 | 0.203917134 | DOWN |
| Test 2 | CD97 | −0.845340799 | 0.045753193 | DOWN |
| Test 2 | CDC14A | −0.646668837 | 0.101888953 | DOWN |
| Test 2 | CDC37 | −0.382406309 | 0.240103288 | DOWN |
| Test 2 | CDC42EP3 | −0.6817877 | 0.19331656 | DOWN |
| Test 2 | CDC42SE1 | −0.378168521 | 0.191525939 | DOWN |
| Test 2 | CDKN1A | −0.387560594 | 0.035341619 | DOWN |
| Test 2 | CEP76 | −0.945072617 | 0.073143922 | DOWN |
| Test 2 | CHD2 | −0.690227101 | 0.069311378 | DOWN |
| Test 2 | CHMP4B | −0.287164907 | 0.094913279 | DOWN |
| Test 2 | CHP1 | −0.291208979 | 0.238519536 | DOWN |
| Test 2 | CLMP | −0.729937574 | 0.225295536 | DOWN |
| Test 2 | CNN2 | −0.512609072 | 0.19331656 | DOWN |
| Test 2 | COTL1 | −0.455952536 | 0.203917134 | DOWN |
| Test 2 | CRKL | −0.30261375 | 0.248133155 | DOWN |
| Test 2 | CSF2RB | −0.678104076 | 0.155367959 | DOWN |
| Test 2 | CSF3R | −0.569585968 | 0.223914394 | DOWN |
| Test 2 | CSNK1G2 | −0.599862624 | 0.204723138 | DOWN |
| Test 2 | CSRNP1 | −0.781500951 | 0.014394956 | DOWN |
| Test 2 | CTSA | −0.33123967 | 0.248133155 | DOWN |
| Test 2 | CTSD | −0.379023691 | 0.240103288 | DOWN |
| Test 2 | CXCL16 | −0.548865027 | 0.188426153 | DOWN |
| Test 2 | CXCR4 | −0.655969209 | 0.097540633 | DOWN |
| Test 2 | CYTH4 | −0.700576961 | 0.184292206 | DOWN |
| Test 2 | DBNL | −0.423838322 | 0.160228921 | DOWN |
| Test 2 | DCAF11 | −0.617731883 | 0.203917134 | DOWN |
| Test 2 | DDX60L | −0.683358582 | 0.227224075 | DOWN |
| Test 2 | DENND5A | −0.668319578 | 0.148123279 | DOWN |
| Test 2 | DGAT2 | −0.360390084 | 0.236147723 | DOWN |
| Test 2 | DHCR24 | −0.591071219 | 0.154704386 | DOWN |
| Test 2 | DIRC2 | −0.516298908 | 0.236575098 | DOWN |
| Test 2 | DSCR3 | −0.542042929 | 0.211019733 | DOWN |
| Test 2 | DUSP1 | −0.541740484 | 0.188426153 | DOWN |
| Test 2 | DUSP2 | −0.689003628 | 0.03912317 | DOWN |

TABLE 4-15

| | | | | |
|---|---|---|---|---|
| Test 2 | DUSP3 | −0.59012828 | 0.122393306 | DOWN |
| Test 2 | DUSP4 | −0.634642504 | 0.235243914 | DOWN |
| Test 2 | ECE1 | −0.65379167 | 0.156533343 | DOWN |
| Test 2 | EFHD2 | −0.617790089 | 0.098631765 | DOWN |
| Test 2 | EFR3A | −0.501652521 | 0.187412757 | DOWN |
| Test 2 | EGR2 | −0.387465028 | 0.179929185 | DOWN |
| Test 2 | EGR3 | −0.721696305 | 0.035341619 | DOWN |
| Test 2 | EHBP1L1 | −0.581774222 | 0.130446509 | DOWN |
| Test 2 | EHD1 | −0.456876838 | 0.240103288 | DOWN |
| Test 2 | EID3 | −0.828295686 | 0.155367959 | DOWN |
| Test 2 | EIF1 | −0.193856817 | 0.240103288 | DOWN |
| Test 2 | EIF4EBP2 | −0.594264701 | 0.02821497 | DOWN |
| Test 2 | EIF4EBP3 | −0.641006049 | 0.240103288 | DOWN |
| Test 2 | ELL | −0.552384988 | 0.184292206 | DOWN |
| Test 2 | EMP3 | −0.54637512 | 0.127634739 | DOWN |
| Test 2 | EPS15L1 | −0.75105164 | 0.088334668 | DOWN |
| Test 2 | FADS2 | −0.634238349 | 0.188426153 | DOWN |
| Test 2 | FAM100B | −0.434221338 | 0.045753193 | DOWN |
| Test 2 | FAM193B | −1.065452843 | 0.039812849 | DOWN |
| Test 2 | FAM213A | −0.619927264 | 0.192977197 | DOWN |
| Test 2 | FAM32A | −0.451599652 | 0.038297933 | DOWN |
| Test 2 | FAM46C | −0.401691761 | 0.203917134 | DOWN |
| Test 2 | FFAR2 | −0.834337354 | 0.129055508 | DOWN |
| Test 2 | FGR | −0.714071655 | 0.074376997 | DOWN |
| Test 2 | FLNA | −0.501907163 | 0.205001407 | DOWN |
| Test 2 | FMNL1 | −0.568342039 | 0.191525939 | DOWN |
| Test 2 | FNIP1 | −0.534504704 | 0.225031103 | DOWN |
| Test 2 | FOSB | −1.497165708 | 0.001079959 | DOWN |
| Test 2 | FOSL2 | −0.650897973 | 0.038524973 | DOWN |
| Test 2 | FURIN | −0.387559873 | 0.083321514 | DOWN |
| Test 2 | GABARAPL1 | −0.427119307 | 0.02821497 | DOWN |
| Test 2 | GADD45B | −0.59479857 | 0.03912317 | DOWN |
| Test 2 | GAL | −0.457031303 | 0.246563533 | DOWN |
| Test 2 | GAS7 | −0.431198926 | 0.183527457 | DOWN |
| Test 2 | GDE1 | −0.423089167 | 0.240103288 | DOWN |
| Test 2 | GPR108 | −0.777015122 | 0.093471839 | DOWN |
| Test 2 | GPR157 | −0.527189631 | 0.101888953 | DOWN |
| Test 2 | GPSM3 | −0.592430722 | 0.191525939 | DOWN |
| Test 2 | GRAMD1A | −0.951769694 | 0.014394956 | DOWN |
| Test 2 | GRINA | −0.595457147 | 0.156533343 | DOWN |
| Test 2 | GRK6 | −0.83349196 | 0.054079361 | DOWN |

TABLE 4-16

| | | | | |
|---|---|---|---|---|
| Test 2 | GRN | −0.535715253 | 0.191525939 | DOWN |
| Test 2 | GTPBP1 | −0.46308194 | 0.148123279 | DOWN |
| Test 2 | HDAC7 | −0.490842046 | 0.248266371 | DOWN |
| Test 2 | HLA-A | −1.104833425 | 0.203917134 | DOWN |
| Test 2 | HPCAL1 | −0.447792645 | 0.216748647 | DOWN |
| Test 2 | HS3ST6 | −0.501663196 | 0.207332199 | DOWN |
| Test 2 | HSPA4 | −0.906581783 | 0.092289404 | DOWN |
| Test 2 | IDS | −0.255674167 | 0.191525939 | DOWN |
| Test 2 | IER3 | −0.441738596 | 0.034346406 | DOWN |
| Test 2 | IMPDH1 | −0.618750853 | 0.202573111 | DOWN |
| Test 2 | INPP5K | −0.39152519 | 0.179929185 | DOWN |
| Test 2 | IRAK2 | −0.941310019 | 0.041102123 | DOWN |
| Test 2 | IRF1 | −0.633328723 | 0.219872648 | DOWN |
| Test 2 | ITGA5 | −0.614100521 | 0.148123279 | DOWN |
| Test 2 | ITGAX | −0.584382378 | 0.191525939 | DOWN |
| Test 2 | ITPK1 | −0.681600843 | 0.167922188 | DOWN |
| Test 2 | JUNB | −0.405633107 | 0.174126215 | DOWN |
| Test 2 | KIAA0247 | −0.43354089 | 0.185751097 | DOWN |
| Test 2 | KIAA0368 | −0.386851905 | 0.149347833 | DOWN |
| Test 2 | KIAA0494 | −0.375961313 | 0.203917134 | DOWN |
| Test 2 | KIAA1191 | −0.759570647 | 0.07612678 | DOWN |
| Test 2 | KLF2 | −0.829622672 | 0.095805056 | DOWN |
| Test 2 | KLF6 | −0.615237069 | 0.032647959 | DOWN |
| Test 2 | LARP1 | −0.371691928 | 0.191525939 | DOWN |
| Test 2 | LGALS3 | −0.33660704 | 0.228358936 | DOWN |
| Test 2 | LILRB2 | −0.713217829 | 0.141991209 | DOWN |
| Test 2 | LILRB3 | −0.586252086 | 0.203917134 | DOWN |
| Test 2 | LIMK2 | −0.693871145 | 0.19331656 | DOWN |
| Test 2 | LITAF | −0.472582053 | 0.129200547 | DOWN |
| Test 2 | LOC146880 | −0.596953376 | 0.248133155 | DOWN |
| Test 2 | LOC729737 | −0.641814422 | 0.204723138 | DOWN |
| Test 2 | LPCAT1 | −1.010875742 | 0.013807457 | DOWN |
| Test 2 | LPIN1 | −0.520806257 | 0.196547493 | DOWN |
| Test 2 | LSP1 | −0.669555347 | 0.066340963 | DOWN |
| Test 2 | LTBR | −0.715104448 | 0.155367959 | DOWN |
| Test 2 | MAF1 | −0.475268842 | 0.232304966 | DOWN |
| Test 2 | MAP4K4 | −0.517139843 | 0.204723138 | DOWN |
| Test 2 | MAP7D1 | −0.449559995 | 0.189823772 | DOWN |
| Test 2 | MAPKAPK2 | −0.475983818 | 0.191525939 | DOWN |
| Test 2 | MARCKS | −0.564491232 | 0.18041519 | DOWN |
| Test 2 | MBOAT7 | −0.751160266 | 0.145937048 | DOWN |

TABLE 4-17

| | | | | |
|---|---|---|---|---|
| Test 2 | MEF2D | −0.626428106 | 0.045753193 | DOWN |
| Test 2 | MEGF9 | −0.362999714 | 0.203917134 | DOWN |
| Test 2 | MEPCE | −0.864615229 | 0.073987946 | DOWN |
| Test 2 | METRNL | −0.281506863 | 0.183513502 | DOWN |
| Test 2 | MGEA5 | −0.345655087 | 0.180032429 | DOWN |
| Test 2 | MKNK2 | −0.410364719 | 0.126316838 | DOWN |
| Test 2 | MLF2 | −0.387393069 | 0.075659228 | DOWN |
| Test 2 | MLLT6 | −0.882491218 | 0.041252415 | DOWN |
| Test 2 | MMP25 | −0.743721149 | 0.204723138 | DOWN |
| Test 2 | MSRB1 | −0.384733834 | 0.160228921 | DOWN |
| Test 2 | MTHFS | −0.57838818 | 0.191525939 | DOWN |
| Test 2 | MTMR14 | −0.690369051 | 0.156533343 | DOWN |
| Test 2 | MYO9B | −0.607502615 | 0.191525939 | DOWN |
| Test 2 | NAA50 | −0.444730906 | 0.019217132 | DOWN |
| Test 2 | NBEAL2 | −0.535965413 | 0.2421585 | DOWN |
| Test 2 | NCF1B | −0.929517474 | 0.094970832 | DOWN |
| Test 2 | NFKB2 | −0.926577772 | 0.023042585 | DOWN |
| Test 2 | NFKBIA | −0.494604251 | 0.189823772 | DOWN |
| Test 2 | NFKBIB | −0.580266689 | 0.221139649 | DOWN |
| Test 2 | NFKBID | −0.861315902 | 0.050427226 | DOWN |
| Test 2 | NFKBIE | −0.730971195 | 0.083321514 | DOWN |
| Test 2 | NINJ1 | −0.82050845 | 0.035341619 | DOWN |
| Test 2 | NIPBL | −0.390839696 | 0.188426153 | DOWN |
| Test 2 | NLRC5 | −0.794366743 | 0.185751097 | DOWN |
| Test 2 | NOTCH2NL | −0.334354965 | 0.094970832 | DOWN |
| Test 2 | NR4A3 | −0.589158721 | 0.249272492 | DOWN |
| Test 2 | NTAN1 | −0.620825777 | 0.126316838 | DOWN |
| Test 2 | OGDH | −0.409053089 | 0.156533343 | DOWN |
| Test 2 | OSM | −0.609522522 | 0.240103288 | DOWN |
| Test 2 | P2RY4 | −0.830041687 | 0.204723138 | DOWN |
| Test 2 | PACSIN2 | −0.430359253 | 0.196505966 | DOWN |
| Test 2 | PDHX | −0.681406483 | 0.24513842 | DOWN |
| Test 2 | PDLIM7 | −0.646265774 | 0.236147723 | DOWN |
| Test 2 | PER1 | −0.715031562 | 0.129200547 | DOWN |
| Test 2 | PFKL | −0.472924949 | 0.189087808 | DOWN |
| Test 2 | PHF1 | −0.616090955 | 0.203917134 | DOWN |
| Test 2 | PIK3AP1 | −0.754938139 | 0.131526721 | DOWN |
| Test 2 | PIK3R5 | −0.699813238 | 0.141991209 | DOWN |
| Test 2 | PILRA | −0.578293645 | 0.221139649 | DOWN |
| Test 2 | PIM2 | −0.543863209 | 0.248133155 | DOWN |
| Test 2 | PIM3 | −0.602922387 | 0.032647959 | DOWN |

TABLE 4-18

| | | | | |
|---|---|---|---|---|
| Test 2 | PITPNA | −0.666509912 | 0.026318959 | DOWN |
| Test 2 | PLAU | −0.978710234 | 0.035341619 | DOWN |
| Test 2 | PLEKHO2 | −0.55687838 | 0.224160094 | DOWN |
| Test 2 | POU5F1P3 | −0.893422599 | 0.204723138 | DOWN |
| Test 2 | PPP1CB | −0.242710496 | 0.184292206 | DOWN |
| Test 2 | PPP1R15A | −0.687500599 | 0.014394956 | DOWN |
| Test 2 | PPP1R18 | −0.70890583 | 0.185751097 | DOWN |
| Test 2 | PPP4R1 | −0.613615567 | 0.130446509 | DOWN |
| Test 2 | PSMF1 | −0.38322124 | 0.228358936 | DOWN |
| Test 2 | PTGER4 | −0.572972237 | 0.179929185 | DOWN |
| Test 2 | PTK2B | −0.43711103 | 0.126316838 | DOWN |
| Test 2 | PTPN6 | −0.506301773 | 0.200987694 | DOWN |
| Test 2 | PTTG1IP | −0.590740401 | 0.185751097 | DOWN |
| Test 2 | RAB11FIP1 | −0.25222007 | 0.17055456 | DOWN |
| Test 2 | RAB20 | −1.092595824 | 0.058926254 | DOWN |
| Test 2 | RAB27A | −0.398774147 | 0.151691535 | DOWN |
| Test 2 | RAB5B | −0.202685445 | 0.229460189 | DOWN |
| Test 2 | RAB5C | −0.423663208 | 0.130446509 | DOWN |
| Test 2 | RALGDS | −1.217008231 | 0.001079959 | DOWN |
| Test 2 | RANGAP1 | −0.552917543 | 0.087496195 | DOWN |
| Test 2 | RAP2A | −0.736519183 | 0.046019666 | DOWN |
| Test 2 | RBCK1 | −0.695750187 | 0.240103288 | DOWN |
| Test 2 | RBM39 | −0.384671412 | 0.207377895 | DOWN |
| Test 2 | RELA | −0.553248888 | 0.10472235 | DOWN |
| Test 2 | RHEB | −0.430417385 | 0.023042585 | DOWN |
| Test 2 | RHOA | −0.306833302 | 0.114612949 | DOWN |
| Test 2 | RHOB | −0.530555714 | 0.138694251 | DOWN |
| Test 2 | RILPL2 | −0.839108918 | 0.094970832 | DOWN |
| Test 2 | RIT1 | −0.681872442 | 0.155367959 | DOWN |
| Test 2 | RNASEK | −0.263726846 | 0.189823772 | DOWN |
| Test 2 | RNF213 | −0.587786601 | 0.203917134 | DOWN |
| Test 2 | RTN4 | −0.471653936 | 0.045753193 | DOWN |
| Test 2 | RXRA | −0.462010218 | 0.094970832 | DOWN |
| Test 2 | RYBP | −0.469450448 | 0.203917134 | DOWN |
| Test 2 | SBNO2 | −0.639964031 | 0.121272078 | DOWN |
| Test 2 | SCARF1 | −0.891605529 | 0.082241574 | DOWN |
| Test 2 | SCD | −0.581730024 | 0.18328675 | DOWN |
| Test 2 | SCYL1 | −0.489185457 | 0.191525939 | DOWN |
| Test 2 | SERINC1 | −0.436066431 | 0.233336584 | DOWN |
| Test 2 | SH2B2 | −0.791605737 | 0.184132179 | DOWN |
| Test 2 | SH3BP5 | −0.614289416 | 0.118903158 | DOWN |

TABLE 4-19

| | | | | |
|---|---|---|---|---|
| Test 2 | SHISA5 | −0.70313086 | 0.200987694 | DOWN |
| Test 2 | SHKBP1 | −0.610047261 | 0.203917134 | DOWN |
| Test 2 | SIRPA | −0.512022506 | 0.03912317 | DOWN |
| Test 2 | SLC11A1 | −0.656308616 | 0.199786172 | DOWN |
| Test 2 | SLC15A3 | −0.637280648 | 0.205001407 | DOWN |
| Test 2 | SLC15A4 | −0.716205845 | 0.130446509 | DOWN |
| Test 2 | SLC31A1 | −0.451679847 | 0.159048563 | DOWN |
| Test 2 | SLC3A2 | −0.828553543 | 0.079852349 | DOWN |
| Test 2 | SLC41A1 | −1.010783773 | 0.079217394 | DOWN |
| Test 2 | SLC43A2 | −0.940136909 | 0.032647959 | DOWN |
| Test 2 | SLC43A3 | −0.690613412 | 0.225295536 | DOWN |
| Test 2 | SLC45A4 | −0.749047969 | 0.093863146 | DOWN |
| Test 2 | SLC6A6 | −0.758400864 | 0.073352416 | DOWN |
| Test 2 | SMG1P1 | −0.693226977 | 0.094970832 | DOWN |
| Test 2 | SNORA8 | −0.519845806 | 0.211019733 | DOWN |
| Test 2 | SORT1 | −0.623031599 | 0.050123349 | DOWN |
| Test 2 | SPHK1 | −1.108424501 | 0.016355202 | DOWN |
| Test 2 | SPINT2 | −0.339670123 | 0.203917134 | DOWN |
| Test 2 | SQSTM1 | −0.276887228 | 0.240103288 | DOWN |
| Test 2 | SREBF2 | −1.227441548 | 0.007293838 | DOWN |
| Test 2 | SRP54 | −0.319771058 | 0.248266371 | DOWN |
| Test 2 | SRRM2 | −0.418303881 | 0.180898511 | DOWN |
| Test 2 | SRXN1 | −0.582979776 | 0.038524973 | DOWN |
| Test 2 | STK40 | −0.488001779 | 0.0381434 | DOWN |
| Test 2 | STX11 | −0.658724054 | 0.205349428 | DOWN |
| Test 2 | STX6 | −0.513619131 | 0.230255192 | DOWN |
| Test 2 | STXBP2 | −0.508585693 | 0.130446509 | DOWN |
| Test 2 | TAGAP | −0.801415208 | 0.118903158 | DOWN |
| Test 2 | TAP1 | −0.654126047 | 0.218710004 | DOWN |
| Test 2 | TCF25 | −0.461070498 | 0.230255192 | DOWN |
| Test 2 | TCIRG1 | −0.804126136 | 0.079217394 | DOWN |
| Test 2 | TECPR2 | −0.825139388 | 0.170236917 | DOWN |
| Test 2 | TEX264 | −0.505490645 | 0.227224075 | DOWN |
| Test 2 | TLE3 | −0.466144984 | 0.203917134 | DOWN |
| Test 2 | TMBIM6 | −0.291260917 | 0.207332199 | DOWN |
| Test 2 | TMEM123 | −0.395242663 | 0.05837947 | DOWN |
| Test 2 | TMEM134 | −0.472439908 | 0.204723138 | DOWN |
| Test 2 | TMEM189 | −0.563615464 | 0.204723138 | DOWN |
| Test 2 | TNFAIP2 | −0.891574563 | 0.035341619 | DOWN |
| Test 2 | TNFRSF14 | −0.722611949 | 0.19331656 | DOWN |
| Test 2 | TNIP1 | −0.425719633 | 0.122393306 | DOWN |

TABLE 4-20

| | | | | |
|---|---|---|---|---|
| Test 2 | TOM1 | −0.505066513 | 0.035341619 | DOWN |
| Test 2 | TPD52L2 | −0.276554324 | 0.248133155 | DOWN |
| Test 2 | TRIB1 | −0.555648849 | 0.183527457 | DOWN |
| Test 2 | TRIM25 | −0.486286342 | 0.216748647 | DOWN |
| Test 2 | TRPC4AP | −0.425236454 | 0.203917134 | DOWN |
| Test 2 | UBAP2L | −0.698597784 | 0.190692011 | DOWN |
| Test 2 | UBE2D3 | −0.400068279 | 0.016355202 | DOWN |
| Test 2 | UBIAD1 | −0.472019436 | 0.203917134 | DOWN |
| Test 2 | UBR4 | −0.790770601 | 0.068265083 | DOWN |
| Test 2 | UCP2 | −0.469130629 | 0.219872648 | DOWN |
| Test 2 | USF2 | −0.455624975 | 0.191525939 | DOWN |
| Test 2 | VOPP1 | −0.479532379 | 0.180898511 | DOWN |
| Test 2 | WBP2 | −0.508317102 | 0.036236726 | DOWN |
| Test 2 | WSB2 | −0.373814864 | 0.183776073 | DOWN |
| Test 2 | XPO6 | −0.62485095 | 0.248489765 | DOWN |
| Test 2 | YKT6 | −0.273245456 | 0.234837927 | DOWN |
| Test 2 | ZC3H12A | −0.776257578 | 0.019217132 | DOWN |
| Test 2 | ZFP36 | −0.505429212 | 0.197195688 | DOWN |
| Test 2 | ZFP36L1 | −0.618017539 | 0.155367959 | DOWN |
| Test 2 | ZHX2 | −0.7205355 | 0.16157383 | DOWN |
| Test 2 | ZMIZ1 | −0.802337523 | 0.079217394 | DOWN |

A biological process (BP) and a KEGG pathway were searched for by gene ontology (GO) enrichment analysis by using the public database STRING. As a result, 30 and 28 KEGG pathways related to the gene group with increased or decreased expression in the PD patients were obtained in Test 1 and Test 2, respectively, and the term hsa05012 (Parkinson's disease) which indicates Parkinson's disease was found to be included in both the tests (Tables 5-1 and 5-2).

TABLE 5-1

| Test | Regulation | ID | Description | FDR |
|---|---|---|---|---|
| Test 1 | UP | hsa05016 | Huntington's disease | 0.0039 |
| Test 1 | UP | hsa04714 | Thermogenesis | 0.0048 |
| Test 1 | UP | hsa00190 | Oxidative | 0.0302 |
| | | | phosphorylation | |
| Test 1 | UP | hsa04932 | Non-alcoholic fatty | 0.0303 |
| | | | liver disease (NAFLD) | |
| Test 1 | UP | hsa05012 | Parkinson's disease | 0.0303 |
| Test 1 | UP | hsa04260 | Cardiac muscle | 0.035 |
| | | | contraction | |
| Test 1 | UP | hsa05010 | Alzheimer's disease | 0.035 |
| Test 1 | UP | hsa01040 | Biosynthesis of | 0.0374 |
| | | | unsaturated fatty acids | |
| Test 1 | UP | hsa05169 | Epstein-Barr | 0.0409 |
| | | | virus infection | |
| Test 1 | UP | hsa04066 | HIF-1 signaling pathway | 0.0422 |
| Test 1 | UP | hsa03020 | RNA polymerase | 0.0472 |
| Test 1 | DOWN | hsa03010 | Ribosome | 0.00000294 |
| Test 1 | DOWN | hsa04144 | Endocytosis | 0.00022 |
| Test 1 | DOWN | hsa05203 | Viral carcinogenesis | 0.00024 |
| Test 1 | DOWN | hsa04670 | Leukocyte | 0.00066 |
| | | | transendothelial | |
| | | | migration | |
| Test 1 | DOWN | hsa05130 | Pathogenic Escherichia | 0.0026 |
| | | | coli infection | |
| Test 1 | DOWN | hsa05323 | Rheumatoid arthritis | 0.0032 |
| Test 1 | DOWN | hsa04141 | Protein processing in | 0.0053 |
| | | | endoplasmic retic | |
| | | | ulum | |
| Test 1 | DOWN | hsa04068 | FoxO signaling pathway | 0.0057 |
| Test 1 | DOWN | hsa05211 | Renal cell carcinoma | 0.0057 |
| Test 1 | DOWN | hsa05168 | Herpes simplex infection | 0.0085 |
| Test 1 | DOWN | hsa05206 | MicroRNAs in cancer | 0.0098 |
| Test 1 | DOWN | hsa04621 | NOD-like receptor | 0.0176 |
| | | | signaling pathway | |
| Test 1 | DOWN | hsa03040 | Spliceosome | 0.018 |
| Test 1 | DOWN | hsa04062 | Chemokine | 0.0255 |
| | | | signaling pathway | |
| Test 1 | DOWN | hsa05100 | Bacterial invasion of | 0.0277 |
| | | | epithelial cells | |
| Test 1 | DOWN | hsa05169 | Epstein-Barr virus | 0.0337 |
| | | | infection | |
| Test 1 | DOWN | hsa04919 | Thyroid hormone | 0.0355 |
| | | | signaling pathway | |
| Test 1 | DOWN | hsa04966 | Collecting duct | 0.0453 |
| | | | acid secretion | |
| Test 1 | DOWN | hsa04140 | Autophagy-animal | 0.0469 |

TABLE 5-2

| Test 2 | UP | hsa03010 | Ribosome | 1.12E−43 |
|---|---|---|---|---|
| Test 2 | UP | hsa00190 | Oxidative | 1.42E−09 |
| | | | phosphorylation | |
| Test 2 | UP | hsa05010 | Alzheimer's disease | 0.000000186 |
| Test 2 | UP | hsa05012 | Parkinson's disease | 0.000000281 |
| Test 2 | UP | hsa04714 | Thermogenesis | 0.000000324 |
| Test 2 | UP | hsa05016 | Huntington's disease | 0.000000399 |
| Test 2 | UP | hsa04932 | Non-alcoholic fatty | 0.0000257 |
| | | | liver disease (NAFLD) | |
| Test 2 | UP | hsa04915 | Estrogen signaling | 0.00075 |
| | | | pathway | |
| Test 2 | UP | hsa04260 | Cardiac muscle | 0.027 |
| | | | contraction | |
| Test 2 | UP | hsa04657 | IL-17 signaling | 0.0453 |
| | | | pathway | |
| Test 2 | UP | hsa04723 | Retrograde | 0.0453 |
| | | | endocannabinoid | |
| | | | signaling | |
| Test 2 | DOWN | hsa04062 | Chemokine | 0.0015 |
| | | | signaling pathway | |
| Test 2 | DOWN | hsa04064 | NF-kappa B | 0.0023 |
| | | | signaling pathway | |
| Test 2 | DOWN | hsa04144 | Endocytosis | 0.0023 |
| Test 2 | DOWN | hsa04380 | Osteoclast | 0.0023 |
| | | | differentiation | |
| Test 2 | DOWN | hsa04722 | Neurotrophin | 0.0041 |
| | | | signaling pathway | |
| Test 2 | DOWN | hsa04920 | Adipocytokine | 0.0041 |
| | | | signaling pathway | |
| Test 2 | DOWN | hsa05152 | Tuberculosis | 0.0041 |
| Test 2 | DOWN | hsa04142 | Lysosome | 0.0042 |
| Test 2 | DOWN | hsa04662 | B cell receptor | 0.0042 |
| | | | signaling pathway | |
| Test 2 | DOWN | hsa05203 | Viral carcinogenesis | 0.0042 |
| Test 2 | DOWN | hsa04218 | Cellular senescence | 0.016 |
| Test 2 | DOWN | hsa04010 | MARK signaling | 0.037 |
| | | | pathway | |
| Test 2 | DOWN | hsa04060 | Cytokine-cytokine | 0.037 |
| | | | receptor interaction | |
| Test 2 | DOWN | hsa04072 | Phospholipase D | 0.037 |
| | | | signaling pathway | |
| Test 2 | DOWN | hsa04145 | Phagosome | 0.037 |
| Test 2 | DOWN | hsa05168 | Herpes simplex | 0.037 |
| | | | infection | |
| Test 2 | DOWN | hsa05222 | Small cell lung cancer | 0.043 |

Previously reported literatures were checked about the relation to Parkinson's disease of the genes shown in Tables 4-1 to 4-20 described above which were differentially expressed in at least either Test 1 or Test 2. As a result, 19 genes shown in Table 6-1 among the genes differentially expressed in Test 1 and 30 genes shown in Table 6-2 among the genes differentially expressed in Test 2 had not been reported so far on their relation to Parkinson's disease, demonstrating that these genes are capable of serving as novel markers for detecting Parkinson's disease. Genes indicated by boldface in the tables are common genes between Test 1 and Test 2.

TABLE 6-1

| Test | Symbol | Regulation |
|---|---|---|
| Test 1 | LOC100288069 | UP |
| Test 1 | MESDC1 | UP |
| Test 1 | POLR2J3 | UP |
| Test 1 | PQLC1 | UP |
| Test 1 | SNORA24 | UP |
| Test 1 | SNORA50 | UP |
| Test 1 | SNORA57 | UP |
| Test 1 | SNORA9 | UP |
| Test 1 | TRMT44 | UP |
| Test 1 | C14orf178 | DOWN |
| Test 1 | FAM100A | DOWN |
| Test 1 | FYTTD1 | DOWN |
| Test 1 | LGALSL | DOWN |
| Test 1 | NSFP1 | DOWN |
| Test 1 | SLMO2 | DOWN |
| Test 1 | SNORA53 | DOWN |
| Test 1 | SREK1IP1 | DOWN |
| Test 1 | SSU72 | DOWN |
| Test 1 | TMEM167B | DOWN |

TABLE 6-2

| Test | Symbol | Regulation |
|---|---|---|
| Test 2 | KRTAP5-3 | UP |
| Test 2 | LRRC15 | UP |
| Test 2 | PINLYP | UP |
| Test 2 | SNORA16A | UP |
| Test 2 | SNORA24 | UP |
| Test 2 | SNORA52 | UP |
| Test 2 | SNORA63 | UP |
| Test 2 | SNORA68 | UP |
| Test 2 | SNORA71A | UP |
| Test 2 | SNORD15B | UP |
| Test 2 | AADACL3 | DOWN |
| Test 2 | ARHGAP30 | DOWN |
| Test 2 | C17orf107 | DOWN |
| Test 2 | C1orf43 | DOWN |
| Test 2 | C22orf13 | DOWN |
| Test 2 | CCDC86 | DOWN |
| Test 2 | CCSAP | DOWN |
| Test 2 | CYTH4 | DOWN |
| Test 2 | FAM100B | DOWN |
| Test 2 | FAM193B | DOWN |
| Test 2 | GPR108 | DOWN |
| Test 2 | GRAMDIA | DOWN |
| Test 2 | KIAA0494 | DOWN |
| Test 2 | KIAA1191 | DOWN |
| Test 2 | LOC729737 | DOWN |
| Test 2 | MAP7D1 | DOWN |
| Test 2 | MLLT6 | DOWN |
| Test 2 | NCF1B | DOWN |
| Test 2 | POU5F1P3 | DOWN |
| Test 2 | SMG1P1 | DOWN |

### Example 2 Preparation and Verification of Discriminant Model—1

1) Data Used

In the data (read count values) on the expression level of SSL-derived RNA from the test subjects, data with a read count of less than 10 was treated as missing values, as in RNA expression analysis—1 in Example 1. After conversion to RPM values which normalized the read count values for difference in the total number of reads among samples, the missing values were compensated for by use of an approach called singular value decomposition (SVD) imputation. However, only genes which produced expression level data without missing values in 80% or more samples in all the samples were used in analysis given below. In the construction of machine learning models, converted RPM values, logarithmic values of RPM value to base 2 (Log₂ RPM values) were used in order to approximate the RPM values, which followed negative binominal distribution, to normal distribution.

2) Data Set Partitioning

In the RNA profile data set obtained from the test subjects of Test 1, RNA profile data from a total of 20 subjects (10 healthy subjects and 10 PD) was used as training data for PD prediction models, and RNA profile data from the remaining 10 subjects was used as test data for use in the evaluation of model precision. In the RNA profile data set obtained from the test subjects of Test 2, RNA profile data from a total of 80 subjects (40 healthy subjects and 40 PD) was used as training data for PD prediction models, and RNA profile data from the remaining 20 subjects was used as test data for use in the evaluation of model precision.

3) Selection of Feature Gene

18 RNAs whose expression was increased in common between Test 1 and Test 2 and 15 RNAs whose expression was decreased in common between Test 1 and Test 2, in the PD patients compared with the healthy subjects in RNA expression analysis—1 in Example 1 (genes indicated by boldface in Tables 1-1 to 1-27) were selected as feature genes. Their expression level data was converted to principal components by principal component analysis. Then, the first to tenth principal components were used as explanatory variables. Among the 18 RNAs whose expression was increased in common between Test 1 and Test 2 and the 15 RNAs whose expression was decreased in common between Test 1 and Test 2 in the PD patients, 4 genes SNORA16A, SNORA24, SNORA50, and REXO1L2P were selected as feature genes. Their expression level data was converted to principal components by principal component analysis. Then, the first to fourth principal components were used as explanatory variables.

4) Model Construction

Prediction model construction was carried out by using a value of each principal component obtained from expression level data (Log₂ RPM values) on the feature genes selected as training data from SSL-derived RNA as an explanatory variable, and the healthy subjects (HL) and PD as objective variables. The prediction models were learned by 10-fold cross validation by using 7 algorithms random forest, linear kernel support vector machine (SVM linear), rbf kernel support vector machine (SVM rbf), neural network, generalized linear model, regularized linear discriminant analysis, and regularized logistic regression for each item to be predicted. As for each algorithm, the value of each principal component obtained from the feature gene expression levels (Log₂ RPM value) of the test data was input to the models thus learned to calculate a target predictive value for each prediction item. Recall, precision, and an F value which is a harmonic mean thereof are calculated from a predictive value and an actually measured value, and a model having the largest F value was selected as the optimum prediction model.

5) Results

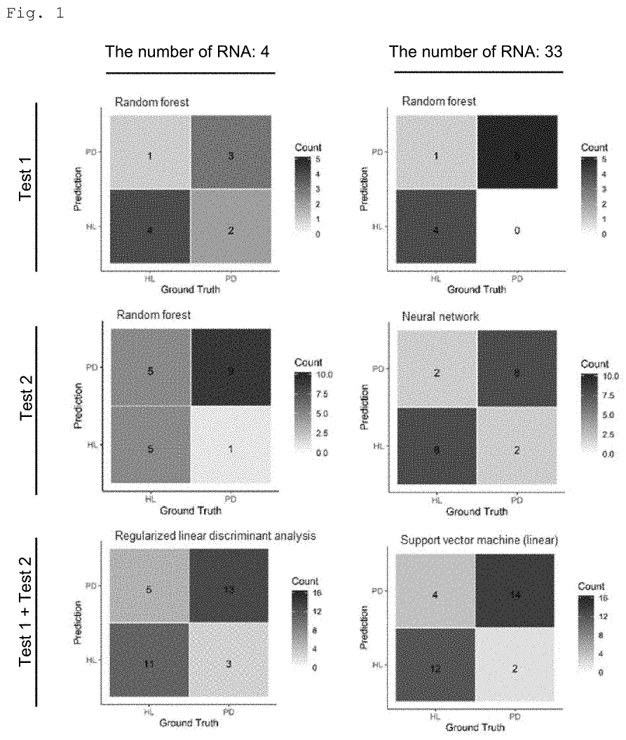

Table 7 shows the algorithm used, the recall, the precision, and the F value of each item to be predicted. FIG. 1 shows confusion matrix in which predictive values in the optimum prediction model and actually measured values were plotted in test data. Numeric values in the drawing represent the number of samples of each quadrant.

Table 8 shows results of calculating the variable importance of each feature gene when random forest was used in model construction.

F1 of the model obtained by using 4 genes SNORA16A, SNORA24, SNORA50, and REXO1L2P was 0.67 in Test 1, 0.75 in Test 2, and 0.76 in integrated Test 1+Test 2, indicating that PD was predictable with this model. F1 of the model obtained by using a total of 33 genes including 18 RNAs with increased expression and 15 RNAs with decreased expression in the PD patients was 0.91 in Test 1, 0.80 in Test 2, and 0.82 in integrated Test 1+Test 2, indicating that PD was more highly accurately predictable with this model.

TABLE 7

| | | | The number of RNA: 4 | | | | | | | The number of RNA: 33 | |
|---|---|---|---|---|---|---|---|---|---|---|---|
| | | | Rf | SMVlinear | SVMrbf | Nnet | GLM | rLDA | rLogistic | Rf | SMVlinear |
| Test 1 | Test data | Precision | 0.75 | 0.6 | 0.6 | 0.5 | 0.5 | 0.67 | 0.5 | 0.83 | 0.83 |
| | | Recall | 0.6 | 0.6 | 0.6 | 0.2 | 0.2 | 0.4 | 0.2 | 1 | 1 |
| | | F-measure | 0.67 | 0.6 | 0.6 | 0.29 | 0.29 | 0.5 | 0.29 | 0.91 | 0.91 |
| | Training | Precision | 1 | 0.83 | 0.91 | 0.91 | 0.91 | 0.82 | 0.9 | 1 | 1 |
| | data | Recall | 1 | 1 | 1 | 1 | 1 | 0.9 | 0.9 | 1 | 1 |
| | | F-measure | 1 | 0.91 | 0.95 | 0.95 | 0.95 | 0.86 | 0.9 | 1 | 1 |
| Test 2 | Test data | Precision | 0.64 | 0.64 | 0.58 | 0.64 | 0.7 | 0.67 | 0.7 | 0.78 | 0.6 |
| | | Recall | 0.9 | 0.7 | 0.7 | 0.7 | 0.7 | 0.8 | 0.7 | 0.7 | 0.6 |
| | | F-measure | 0.75 | 0.67 | 0.64 | 0.67 | 0.7 | 0.73 | 0.7 | 0.74 | 0.6 |
| | Training | Precision | 1 | 0.76 | 0.79 | 0.78 | 0.77 | 0.74 | 0.71 | 1 | 0.74 |
| | data | Recall | 1 | 0.78 | 0.83 | 0.88 | 0.75 | 0.78 | 0.63 | 1 | 0.65 |
| | | F-measure | 1 | 0.77 | 0.8 | 0.82 | 0.76 | 0.76 | 0.67 | 1 | 0.69 |
| Test 1 + | Test data | Precision | 0.53 | 0.68 | 0.61 | 0.63 | 0.71 | 0.72 | 0.54 | 0.74 | 0.78 |
| Test 2 | | Recall | 0.56 | 0.81 | 0.69 | 0.75 | 0.75 | 0.81 | 0.44 | 0.88 | 0.88 |
| | | F-measure | 0.55 | 0.74 | 0.65 | 0.69 | 0.73 | 0.76 | 0.48 | 0.8 | 0.82 |
| | Training | Precision | 1 | 0.66 | 0.77 | 0.71 | 0.69 | 0.68 | 0.53 | 1 | 0.84 |
| | data | Recall | 1 | 0.71 | 0.82 | 0.92 | 0.67 | 0.65 | 0.37 | 1 | 0.86 |
| | | F-measure | 1 | 0.69 | 0.79 | 0.8 | 0.68 | 0.67 | 0.43 | 1 | 0.85 |
| | | | | | | | The number of RNA: 33 | | | | |
| | | | | | | | SVMrbf | Nnet | GLM | rLDA | rLogistic |

| | | | | | | | |
|---|---|---|---|---|---|---|---|
| Test 1 | Test data | Precision | 0.83 | 0.83 | 0.83 | 0.83 | 0.83 |
| | | Recall | 1 | 1 | 1 | 1 | 1 |
| | | F-measure | 0.91 | 0.91 | 0.91 | 0.91 | 0.91 |
| | Training | Precision | 1 | 1 | 1 | 1 | 1 |
| | data | Recall | 1 | 1 | 1 | 1 | 1 |
| | | F-measure | 1 | 1 | 1 | 1 | 1 |
| Test 2 | Test data | Precision | 0.69 | 0.8 | 0.67 | 0.6 | 0.69 |
| | | Recall | 0.9 | 0.8 | 0.6 | 0.6 | 0.9 |
| | | F-measure | 0.78 | 0.8 | 0.63 | 0.6 | 0.78 |
| | Training | Precision | 0.97 | 1 | 0.76 | 0.77 | 0.73 |
| | data | Recall | 0.95 | 1 | 0.73 | 0.68 | 0.75 |
| | | F-measure | 0.96 | 1 | 0.74 | 0.72 | 0.74 |
| Test 1 + | Test data | Precision | 0.78 | 0.74 | 0.78 | 0.78 | 0.76 |
| Test 2 | | Recall | 0.88 | 0.88 | 0.88 | 0.88 | 0.81 |
| | | F-measure | 0.82 | 0.8 | 0.82 | 0.82 | 0.79 |
| | Training | Precision | 0.92 | 1 | 0.82 | 0.8 | 0.78 |
| | data | Recall | 0.94 | 1 | 0.82 | 0.84 | 0.82 |
| | | F-measure | 0.93 | 1 | 0.82 | 0.82 | 0.8 |

*Rf, random forest; SVMlinear, linear kernel support vector machine; SVMrbf, rbf kernel support vector machine; Nnet, neural network; GLM, generalized linear model; rLDA, regularized linear discriminant analysis; rLogistic, regularized logistic regression

TABLE 8

| The number of feature RNA: 4 | | The number of feature RNA: 33 | |
|---|---|---|---|
| Gene | Importance | Gene | Importance |
| SNORA16A | 0.280469095 | EGR2 | 0.121039691 |
| SNORA24 | 0.274323927 | RHOA | 0.113763948 |
| SNORA50 | 0.24669339 | CCNI | 0.093092191 |
| REXO1L2P | 0.198513588 | RNASEK | 0.063837117 |

| | |
|---|---|
| CSF2RB | 0.048802707 |
| SERP1 | 0.048409696 |
| ANKRD12 | 0.045938856 |
| SLC25A3 | 0.041588563 |
| SNORA16A | 0.039001187 |
| CD83 | 0.030624415 |
| CXCR4 | 0.027441137 |
| ITGAX | 0.026515533 |
| UQCRH | 0.024491485 |
| SNORA24 | 0.024265663 |
| KCNQ1OT1 | 0.022758123 |
| CCL3 | 0.022737515 |
| C10orf116 | 0.018907367 |
| SERPINB4 | 0.018665702 |
| LCE3D | 0.01686108 |
| CNFN | 0.016538758 |
| SNORA50 | 0.015782887 |
| CNN2 | 0.013610312 |
| SNRPG | 0.012844074 |
| SRRM2 | 0.012694083 |
| RPL7A | 0.012650305 |
| NDUFA4L2 | 0.012282458 |
| RPS26 | 0.011473664 |
| REXO1L2P | 0.007799926 |
| EMP1 | 0.007547062 |
| POLR2L | 0.00754434 |
| SERINC1 | 0.007300344 |
| NDUFS5 | 0.006761863 |
| LITAF | 0.006427944 |

### Example 3 Preparation and Verification of Discriminant Model—2

1) Data Used

Data (read count values) on the expression level of SSL-derived RNA from the test subjects was normalized by use of an approach called DESeq2, as in RNA expression analysis—2 in Example 1. However, a sample in which 4161 or more genes were not detected was excluded, and only genes which produced expression level data without missing values in 90% or more sample test subjects in the expression level data on the test subjects in all the samples after exclusion were used in analysis given below. In the analysis, normalized count values obtained by use of an approach called DESeq2 were used.

2) Data Set Partitioning

In the RNA profile data set obtained from the test subjects of Test 1, RNA profile data from a total of 15 subjects (9 healthy subjects and 6 PD) was used as training data for PD prediction models, and RNA profile data from a total of 5 subjects (the remaining 4 healthy subjects and 1 PD) was used as test data for use in the evaluation of model precision. In the RNA profile data set obtained from the test subjects of Test 2, RNA profile data from a total of 72 subjects (37 healthy subjects and 35 PD) was used as training data for PD prediction models, and RNA profile data from a total of 24 subjects (the remaining 13 healthy subjects and 11 PD) was used as test data for use in the evaluation of model precision.

3) Selection of Feature Gene

17 RNAs whose expression was increased or decreased in common between Test 1 and Test 2 in the PD patients compared with the healthy subjects in RNA expression analysis—2 in Example 1 (genes indicated by boldface in Tables 4-1 to 4-20) were selected as feature genes. Their expression level data was converted to principal components by principal component analysis. Then, the first to fourth principal components were used as explanatory variables.

4) Model Construction

Prediction model construction was carried out by using a value of each principal component obtained from expression level data (logarithmic values to base 2 of normalized count values plus 1) on the feature genes selected as training data from SSL-derived RNA as an explanatory variable, and the healthy subjects (HL) and PD as objective variables. The prediction models were learned by 10-fold cross validation by using 7 algorithms random forest, linear kernel support vector machine (SVM linear), rbf kernel support vector machine (SVM rbf), neural network, generalized linear model, regularized linear discriminant analysis, and regularized logistic regression for each item to be predicted. As for each algorithm, the value of each principal component obtained from the feature gene expression levels (logarithmic values to base 2 of normalized count values plus 1) of the test data was input to the models thus learned to calculate a target predictive value for each prediction item. Recall, precision, and an F value which is a harmonic mean thereof are calculated from a predictive value and an actually measured value, and a model having the largest F value was selected as the optimum prediction model.

5) Results

Table 9 shows the algorithm used, the recall, the precision, and the F value of each item to be predicted.

The F value of the model obtained by using 17 RNAs whose expression was increased or decreased in common between Test 1 and Test 2 in results of the likelihood ratio test after normalization by DESeq2 was 1 in Test 1 and 0.87 in Test 2, indicating that PD was predictable with this model.

TABLE 9

| | | | The number of RNA: 17 | | | | | | |
|---|---|---|---|---|---|---|---|---|---|
| | | | Rf | SMVlinear | SVMrbf | Nnet | GLM | rLDA | rLogistic |
| Test 1 | Test data | Precision | 1 | 1 | 1 | 1 | 1 | 1 | 1 |
| | | Recall | 1 | 1 | 1 | 1 | 1 | 1 | 1 |
| | | F-measure | 1 | 1 | 1 | 1 | 1 | 1 | 1 |
| | Training | Precision | 1 | 0.86 | 1 | 1 | 1 | 0.86 | 0.86 |
| | data | Recall | 1 | 1 | 1 | 1 | 1 | 1 | 1 |
| | | F-measure | 1 | 0.92 | 1 | 1 | 1 | 0.92 | 0.92 |
| Test 2 | Test data | Precision | 0.83 | 0.57 | 0.55 | 0.71 | 0.6 | 0.6 | 0.62 |
| | | Recall | 0.91 | 0.73 | 0.55 | 0.45 | 0.82 | 0.82 | 0.73 |
| | | F-measure | 0.87 | 0.64 | 0.55 | 0.56 | 0.69 | 0.69 | 0.67 |
| | Training | Precision | 1 | 0.77 | 0.84 | 1 | 0.74 | 0.74 | 0.77 |
| | data | Recall | 1 | 0.66 | 0.77 | 1 | 0.66 | 0.66 | 0.66 |
| | | F-measure | 1 | 0.71 | 0.81 | 1 | 0.7 | 0.7 | 0.71 |

*Rf, random forest; SVMlinear, linear kernel support vector machine; SVMrbf, rbf kernel support vector machine; Nnet, neural network; GLM, generalized linear model; rLDA, regularized linear discriminant analysis; rLogistic, regularized logistic regression

### Example 4 Preparation and Verification of Discriminant Model—3

1) Data Used

Data (read count values) on the expression level of SSL-derived RNA from the test subjects was normalized by use of an approach called DESeq2, as in RNA expression analysis—2 in Example 1. However, a sample in which 4161 or more genes were not detected was excluded, and only genes which produced expression level data without missing values in 90% or more sample test subjects in the expression level data on the test subjects in all the samples after exclusion were used in analysis given below. In the analysis, normalized count values obtained by use of an approach called DESeq2 were used.

2) Data Set Partitioning

In the RNA profile data set obtained from the test subjects of Test 1, RNA profile data from a total of 15 subjects (9 healthy subjects and 6 PD) was used as training data for PD prediction models, and RNA profile data from a total of 5 subjects (the remaining 4 healthy subjects and 1 PD) was used as test data for use in the evaluation of model precision. In the RNA profile data set obtained from the test subjects of Test 2, RNA profile data from a total of 72 subjects (37 healthy subjects and 35 PD) was used as training data for PD prediction models, and RNA profile data from a total of 24 subjects (the remaining 13 healthy subjects and 11 PD) was used as test data for use in the evaluation of model precision.

3) Selection of Feature Gene

19 RNAs whose expression was increased or decreased in Test 1 in the PD patients compared with the healthy subjects (genes shown in Table 6-1) or 30 RNAs whose expression was increased or decreased in Test 2 in the PD patients compared with the healthy subjects (genes shown in Table 6-2) in RNA expression analysis—2 in Example 1 were selected as feature genes. Their expression level data was converted to principal components by principal component analysis. Then, the first to fourth principal components were used as explanatory variables.

4) Model Construction

Prediction model construction was carried out by using a value of each principal component obtained from expression level data (logarithmic values to base 2 of normalized count values plus 1) on the feature genes selected as training data from SSL-derived RNA as an explanatory variable, and the healthy subjects (HL) and PD as objective variables. The prediction models were learned by 10-fold cross validation by using 7 algorithms random forest, linear kernel support vector machine (SVM linear), rbf kernel support vector machine (SVM rbf), neural network, generalized linear model, regularized linear discriminant analysis, and regularized logistic regression for each item to be predicted. As for each algorithm, the value of each principal component obtained from the feature gene expression levels (logarithmic values to base 2 of normalized count values plus 1) of the test data was input to the models thus learned to calculate a target predictive value for each prediction item. Recall, precision, and an F value which is a harmonic mean thereof are calculated from a predictive value and an actually measured value, and a model having the largest F value was selected as the optimum prediction model.

5) Results

Tables 10 and 11 show the algorithm used, the recall, the precision, and the F value of each item to be predicted.

The F value of the model obtained by using 19 RNAs whose relation to Parkinson's disease had not been reported so far among RNAs whose expression was increased or decreased in results of the likelihood ratio test after normalization by DESeq2 in Test 1 was 1, indicating that PD was predictable with this model. The F value of the model obtained by using 30 RNAs whose relation to Parkinson's disease had not been reported so far among RNAs whose expression was increased or decreased in results of the likelihood ratio test after normalization by DESeq2 in Test 2 was 0.87, indicating that PD was predictable with this model.

TABLE 10

| | | | The number of RNA: 19 | | | | | | |
|---|---|---|---|---|---|---|---|---|---|
| | | | Rf | SMVlinear | SVMrbf | Nnet | GLM | rLDA | rLogistic |
| Test 1 | Test data | Precision | 1 | 1 | 1 | 1 | 1 | 1 | 1 |
| | | Recall | 1 | 1 | 1 | 1 | 1 | 1 | 1 |
| | | F-measure | 1 | 1 | 1 | 1 | 1 | 1 | 1 |
| | Training data | Precision | 1 | 1 | 1 | 1 | 1 | 1 | 1 |
| | | Recall | 1 | 1 | 1 | 1 | 1 | 1 | 0.83 |
| | | F-measure | 1 | 1 | 1 | 1 | 1 | 1 | 0.91 |

*Rf, random forest; SVMlinear, linear kernel support vector machine; SVMrbf, rbf kernel support vector machine; Nnet, neural network; GLM, generalized linear model; rLDA, regularized linear discriminant analysis; rLogistic, regularized logistic regression

TABLE 11

| | | | The number of RNA: 30 | | | | | | |
|---|---|---|---|---|---|---|---|---|---|
| | | | Rf | SMVlinear | SVMrbf | Nnet | GLM | rLDA | rLogistic |
| Test 2 | Test data | Precision | 0.83 | 0.82 | 0.77 | 0.82 | 0.83 | 0.82 | 0.82 |
| | | Recall | 0.91 | 0.82 | 0.91 | 0.82 | 0.91 | 0.82 | 0.82 |
| | | F-measure | 0.87 | 0.82 | 0.83 | 0.82 | 0.87 | 0.82 | 0.82 |
| | Training data | Precision | 1 | 0.81 | 0.82 | 0.81 | 0.83 | 0.83 | 0.81 |
| | | Recall | 1 | 0.86 | 0.89 | 0.83 | 0.83 | 0.86 | 0.86 |
| | | F-measure | 1 | 0.83 | 0.85 | 0.82 | 0.83 | 0.85 | 0.83 |

*Rf, random forest; SVMlinear, linear kernel support vector machine; SVMrbf, rbf kernel support vector machine; Nnet, neural network; GLM, generalized linear model; rLDA, regularized linear discriminant analysis; rLogistic, regularized logistic regression

### Example 5 Preparation and Verification of Discriminant Model—4

1) Data Used

In the data (read count values) on the expression level of SSL-derived RNA from the test subjects, data with a read count of less than 10 was treated as missing values, as in RNA expression analysis—1 in Example 1. After conversion to RPM values which normalized the read count values for difference in the total number of reads among samples, the missing values were compensated for by use of an approach called singular value decomposition (SVD) imputation. However, only genes which produced expression level data without missing values in 80% or more samples in all the samples were used in analysis given below. In the construction of machine learning models, converted RPM values, logarithmic values of RPM value to base 2 (Log₂ RPM values) were used in order to approximate the RPM values, which followed negative binominal distribution, to normal distribution.

2) Data Set Partitioning

In the RNA profile data set obtained from the test subjects of Test 1, RNA profile data from a total of 20 subjects (10 healthy subjects and 10 PD) was used as training data for PD prediction models, and RNA profile data from the remaining 10 subjects was used as test data for use in the evaluation of model precision. In the RNA profile data set obtained from the test subjects of Test 2, RNA profile data from a total of 80 subjects (40 healthy subjects and 40 PD) was used as training data for PD prediction models, and RNA profile data from the remaining 20 subjects was used as test data for use in the evaluation of model precision.

3) Selection of Feature Gene

21 RNAs whose expression was increased or decreased in Test 1 in the PD patients compared with the healthy subjects (genes shown in Table 3-1) or 92 RNAs whose expression was increased or decreased in Test 2 in the PD patients compared with the healthy subjects (genes shown in Tables 3-2 to 3-4) in RNA expression analysis—1 in Example 1 were selected as feature genes. Their expression level data was converted to principal components by principal component analysis. Then, the first to fourth principal components were used as explanatory variables.

4) Model Construction

Prediction model construction was carried out by using a value of each principal component obtained from expression level data (Log₂ RPM values) on the feature genes selected as training data from SSL-derived RNA as an explanatory variable, and the healthy subjects (HL) and PD as objective variables. The prediction models were learned by 10-fold cross validation by using 7 algorithms random forest, linear kernel support vector machine (SVM linear), rbf kernel support vector machine (SVM rbf), neural network, generalized linear model, regularized linear discriminant analysis, and regularized logistic regression for each item to be predicted. As for each algorithm, the value of each principal component obtained from the feature gene expression levels (Log₂ RPM value) of the test data was input to the models thus learned to calculate a target predictive value for each prediction item. Recall, precision, and an F value which is a harmonic mean thereof are calculated from a predictive value and an actually measured value, and a model having the largest F value was selected as the optimum prediction model.

5) Results

Tables 12 and 13 show the algorithm used, the recall, the precision, and the F value of each item to be predicted.

The F value of the model obtained by using 21 RNAs whose relation to Parkinson's disease had not been reported so far among RNAs whose expression was increased or decreased in results of the test after normalization by Log₂ RPM in Test 1 was 0.91, indicating that PD was predictable with this model. The F value of the model obtained by using 92 RNAs whose relation to Parkinson's disease had not been reported so far among RNAs whose expression was increased or decreased in results of the test after normalization by Log₂ RPM in Test 2 was 0.9, indicating that PD was predictable with this model.

TABLE 12

| | | | The number of RNA: 21 | | | | | | |
|---|---|---|---|---|---|---|---|---|---|
| | | | Rf | SMVlinear | SVMrbf | Nnet | GLM | rLDA | rLogistic |
| Test 1 | Test data | Precision | 0.83 | 0.75 | 0.71 | 0.8 | 0.75 | 0.8 | 0.8 |
| | | Recall | 1 | 0.6 | 1 | 0.8 | 0.6 | 0.8 | 0.8 |
| | | F-measure | 0.91 | 0.67 | 0.83 | 0.8 | 0.67 | 0.8 | 0.8 |
| | Training data | Precision | 1 | 1 | 1 | 1 | 1 | 1 | 1 |
| | | Recall | 1 | 0.9 | 1 | 1 | 1 | 1 | 1 |
| | | F-measure | 1 | 0.95 | 1 | 1 | 1 | 1 | 1 |

*Rf, random forest; SVMlinear, linear kernel support vector machine; SVMrbf, rbf kernel support vector machine; Nnet, neural network; GLM, generalized linear model; rLDA, regularized linear discriminant analysis; rLogistic, regularized logistic regression

TABLE 13

| | | | The number of RNA: 92 | | | | | | |
|---|---|---|---|---|---|---|---|---|---|
| | | | Rf | SMVlinear | SVMrbf | Nnet | GLM | rLDA | rLogistic |
| Test 2 | Test data | Precision | 0.9 | 0.88 | 0.88 | 0.88 | 0.88 | 0.88 | 0.89 |
| | | Recall | 0.9 | 0.7 | 0.7 | 0.7 | 0.7 | 0.7 | 0.8 |
| | | F-measure | 0.9 | 0.78 | 0.78 | 0.78 | 0.78 | 0.78 | 0.84 |
| | Training data | Precision | 1 | 0.83 | 0.92 | 0.92 | 0.87 | 0.83 | 0.89 |
| | | Recall | 1 | 0.83 | 0.88 | 0.85 | 0.85 | 0.88 | 0.83 |
| | | F-measure | 1 | 0.83 | 0.9 | 0.88 | 0.86 | 0.85 | 0.86 |

*Rf, random forest; SVMlinear, linear kernel support vector machine; SVMrbf, rbf kernel support vector machine; Nnet, neural network; GLM, generalized linear model; rLDA, regularized linear discriminant analysis; rLogistic, regularized logistic regression

1. A method for detecting Parkinson's disease in a test subject, comprising a step of measuring an expression level of at least one gene selected from the group of 4 genes consisting of SNORA16A, SNORA24, SNORA50 and REXO1L2P or an expression product thereof in skin surface lipids collected from the test subject.

2. The method for detecting Parkinson's disease according to claim 1, wherein the method at least comprises measuring an expression level of SNORA24 gene or an expression product thereof.

3. The method according to claim 1, wherein the expression level of the gene or the expression product thereof is measured as an expression level of mRNA.

4. (canceled)

5. The method according to claim 1, wherein the presence or absence of Parkinson's disease is evaluated by comparing the measurement value of the expression level with a reference value of the gene or the expression product thereof.

6. The method according to claim 1, wherein the presence or absence of Parkinson's disease in the test subject is evaluated by the following steps: preparing a discriminant which discriminates between the Parkinson's disease patient and a healthy person by using measurement values of an expression level of the gene or the expression product thereof derived from a Parkinson's disease patient and an expression level of the gene or the expression product thereof derived from a healthy subject as teacher samples; substituting the measurement value of the expression level of the gene or the expression product thereof obtained from the biological sample collected from the test subject into the discriminant; and comparing the obtained results with a reference value.

7. The method according to claim 6, wherein expression levels of all the genes of the group of 4 genes or expression products thereof are measured.

8. The method according to claim 6, wherein expression levels of the at least one gene selected from the group of 4 genes as well as at least one gene selected from the following group of 29 genes or expression products thereof are measured:
ANKRD12, C10orf116, CCL3, CCNI, CD83, CNFN, CNN2, CSF2RB, CXCR4, EGR2, EMP1, ITGAX, KCNQ1OT1, LCE3D, LITAF, NDUFA4L2, NDUFS5, POLR2L, RHOA, RNASEK, RPL7A, RPS26, SERINC1, SERP1, SERPINB4, SLC25A3, SNRPG, SRRM2, and UQCRH.

9. The method according to claim 8, wherein expression levels of the at least one gene selected from the group of 4 genes as well as at least one gene selected from the following group of 10 genes or expression products thereof are measured:
CCL3, CCNI, CXCR4, EGR2, EMP1, POLR2L, RHOA, RNASEK, SERINC1, and SERPINB4.

10. The method according to claim 6, wherein expression levels of the at least one gene selected from the group of 4 genes as well as at least one gene selected from the groups of 1,005 genes shown in the following Tables 1-1 to 1-27 and 725 genes shown in the following Tables 4-1 to 4-20 except for the 4 genes, or expression products thereof are measured
TABLE 1-1
ADRM1
ARF5
ARHGEF5
BCKDK
C10orf116
C11orf10
C14orf2
CEBPA
CHAC1
CHCHD2
CMIP
CNFN
COPE
COPS8
COX8A
CSDA
CTBP2
CTDNEP1
CYFIP1
DAD1
DNASE1L2
DUX4L4
EDF1
EIF3E
EIF4G1
EMP1
FAM129B
FAM83G
FEM1B
G6PD
GPBP1L1
GPR157
GPX3
HECA
HIPK1
HIST2H2BE
HLA.DQB2
HMGCS1
HSPA1A
IQSEC1
KCNQ1OT1
TABLE 1-2
KCTD11
KIAA0930
KLHDC3
LCE3D
LOC100093631
LOC100506888
LOC349196
LOC401321
LPIN1
MAP2K2
METRNL
MGLL
NDUFA13
NDUFA4L2
NDUFB11
NDUFS5
NR4A3
OAZ1
OR4F3
PKP3
POLD4
POLR2L
PPA1
PQLC1
PRELID1
PSMB7
PSMC1
PSMD4
PURB
RAP2B
RASAL1
REXO1L2P
RPL7A
RPS26
RRAD
RRAGA
SEC61A1
SERPINB4
SFXN3
TABLE 1-3
SLC25A3
SNF8
SNORA16A
SNORA24
SNORA43
SNORA50
SNORA8
SNRPG
SPINT1
SQRDL
SRXN1
STAT6
STIP1
TALDO1
TCEB3CL
TCIRG1
TEX264
TMEM183A
TRMT112
TTC9
TYMP
UQCRB
UQCRC1
UQCRH
UQCRQ
USP17L5
USP17L6P
USP38
VEGFA
ZNF33A
ZNF410
TABLE 1-4
ACSL1
ACSL4
ANKRD12
ARPC1B
BRD4
BTG1
CALM2
CCL3
CCNI
CD83
CDC42
CHMP4B
CNBP
CNN2
CSF2RB
CXCR4
DDX5
EEF1A1
EEF1B2
EGR2
EIF1
EPS15
GNG10
GRINA
H3F3A
HIF1A
HNRNPA2B1
HNRNPU
IFNGR2
IL1RN
ITGAX
LITAF
LYN
NEAT1
PABPC1
PAIP2
PGK1
PLXNC1
RABGEF1
RAP1A
REL
TABLE 1-5
RGS2
RHOA
RNASEK
RPL10
RPL15
RPL19
RPL21
RPL26
RPL28
RPL3
RPL30
RPL35
RPL5
RPL6
RPS20
RPS25
S100A11
SCARNA9
SERINC1
SERP1
SNORA53
SRRM2
STK24
TMEM127
TNIP1
TPM4
TPT1
TABLE 1-6
A2ML1
ABRACL
ACBD3
ACOT13
ACSS3
ADAP2
ADPRHL2
ADSL
ADSS
AHCY
AIF1L
AIM1L
AK1
AK4
ALDH1A3
ALDOC
AMBRA1
ANP32B
ANP32E
ANXA1
AP4S1
ARFGAP2
ARHGAP29
ARL1
ASS1
ATP5B
ATP5E
ATP5G1
ATP5I
ATP5O
ATPIF1
BAG3
BCAS1
BCAS2
BCL2L13
BCL7C
BMP2
C10orf116
C11orf31
C1orf52
C1orf63
TABLE 1-7
C22orf32
C2orf49
C5orf43
C5orf46
C8orf33
CACYBP
CALM1
CARHSP1
CASK
CASP14
CAST
CCDC6
CCNE1
CCT2
CCT3
CCT4
CCT8
CDC16
CDSN
CGA
CGNL1
CHI3L2
CHIC2
CHMP4A
CIZ1
CKB
CLIC3
CLIP1
CNDP2
CNFN
CNIH4
CNN3
CNNM4
COA1
COA3
COMT
COX4I1
COX5B
CPEB2
CPNE3
CRABP2
TABLE 1-8
CRELD2
CRIPT
CRNN
CST6
CSTA
CUL4A
CUTA
CYB5A
CYB5B
DANCR
DCAF12
DDRGK1
DDT
DEGS1
DENND2C
DHPS
DHX29
DHX32
DHX40
DNAJA1
DNAJA4
DNAJC13
DNAJC15
DNAJC21
DNAJC7
DNAJC9
DOCK6
DOCK9
DPH1
DPY30
DRG1
DSG1
DUSP11
DYM
DYNC1LI1
DYNLL1
DYNLRB1
ECHS1
EFNB2
EIF1AX
EIF2S2
TABLE 1-9
EIF3K
EIF4EBP1
ELOVL7
EMP1
ENDOD1
EPHB6
EPHX3
ERBB3
ERO1L
EXOC4
EXOC5
EXOC6B
F13A1
FABP4
FABP9
FAM108B1
FAM135A
FAM210B
FAM25B
FAM3C
FAM45A
FAM46B
FBXO45
FCHSD1
FIG4
FKBP1A
FKBP3
FLG
FOXQ1
FRMD6
FTSJ1
FUNDC2
FYN
GBAS
GGCT
GHITM
GLOD4
GNL3
GPSM2
GRHL3
GRPEL1
TABLE 1-10
GTF2A2
GTF2E2
GTF2H5
GTF3C5
GTF3C6
H1FX
HADH
HBEGF
HDAC1
HDDC2
HEATR5A
HEXB
HIBADH
HIBCH
HIST1H1E
HIST1H2AE
HIST1H2AG
HIST1H2AI
HIST1H2AM
HIST1H2BN
HIST1H3B
HIST1H3I
HIST1H4B
HIST1H4E
HIST1H4F
HIST1H4H
HMOX2
HNRNPA0
HOMER1
HOOK1
HPGD
HRSP12
HSD17B10
HSP90AA1
HSPD1
HYPK
IDE
IDH3A
IFI27
IL32
IL36A
TABLE 1-11
ILKAP
IPO5
IQCG
ITGB1BP1
ITPA
ITPRIPL2
IVL
KANK1
KCNQ1OT1
KIAA0240
KIAA1143
KLF5
KLK13
KLK7
KLK8
KRT14
KRT16
KRT25
KRT26
KRT27
KRT5
KRT6A
KRT6C
KRT71
KRT72
KRT74
KRT78
KRTAP1.5
KRTAP12.1
KRTAP12.2
KRTAP19.1
KRTAP3.1
KRTAP3.3
KRTAP5.3
KRTAP5.7
KRTDAP
KTN1
LCE2A
LCE2C
LCE2D
LCE3D
TABLE 1-12
LCE3E
LCMT1
LCN2
LEMD3
LEPROTL1
LINC00675
LLPH
LMBR1
LNX1
LOC100505738
LOC550643
LOC646862
LRBA
LRRC15
LSM10
LSM2
LSM7
LTF
LY6D
LYNX1
MAFA
MAL
MALL
MAOA
MAP4K3
MAP7
MCCC1
MCTS1
MICALCL
MNF1
MPHOSPH6
MPV17
MRPL11
MRPL12
MRPL24
MRPL32
MRPL47
MRPS11
MRPS18B
MRPS24
MT1X
TABLE 1-13
MTMR12
MUT
MYO10
MZT2A
NCBP2
NCK1
NDRG2
NDUFA12
NDUFA2
NDUFA4L2
NDUFB1
NDUFS5
NDUFS6
NEDD4L
NFU1
NHP2
NIN
NIPAL3
NIPAL4
NOSIP
NRIP3
NSMCE1
NUDC
NUMA1
NUP214
NUPL1
OFD1
OLA1
ORMDL3
PABPN1
PADI1
PADI3
PAK4
PAPL
PCCB
PDCD5
PDDC1
PDE12
PDHA1
PDZD8
PDZK1IP1
TABLE 1-14
PEPD
PFDN2
PFDN5
PFDN6
PHAX
PHF13
PHPT1
PICK1
PINLYP
PITRM1
PKP1
PLCD1
PLD2
PLS3
POF1B
POLR2D
POLR2G
POLR2L
POLR2M
PPFIBP2
PPID
PPIL4
PPL
PPP1R13B
PPP2R2A
PPP5C
PPWD1
PRDX3
PRDX6
PREP
PRKRA
PROM2
PRPF40A
PRPF4B
PRR9
PRSS3
PSMC2
PSORS1C2
PTPN3
PVRL4
QKI
TABLE 1-15
RAB38
RABIF
RANBP1
RANBP10
RARRES1
RBM10
RBMS2
REXO1L2P
RHCG
RMRP
RNASE7
RNF121
RNF20
ROMO1
RPA1
RPIA
RPL10A
RPL18
RPL21
RPL26L1
RPL30
RPL32
RPL36
RPL36A
RPL37A
RPL38
RPL7
RPL7A
RPLP0
RPLP1
RPS12
RPS15
RPS15A
RPS18
RPS26
RPS28
RPS29
RPS3
RPS4X
RPS5
RPS6
TABLE 1-16
RPS6KA2
RPS6KB1
RPTN
S100A14
S100A7
S100A7A
S100A8
S100A9
SBDS
SBF1
SBSN
SCARNA12
SCARNA16
SCARNA17
SCARNA6
SCARNA7
SCGB2A2
SCNN1B
SCNN1G
SDR16C5
SDR9C7
SEC23A
SERPINA9
SERPINB4
SERPINB5
SERPINB7
SF3B14
SF3B3
SH3GL3
SLC10A6
SLC25A20
SLC25A3
SLC25A5
SLC26A9
SLC5A1
SLC6A14
SLC6A8
SLFN5
SLMO2
SLURP1
SMAD7
TABLE 1-17
SMC3
SMEK2
SMIM5
SNHG1
SNHG16
SNHG6
SNHG9
SNIP1
SNORA10
SNORA14B
SNORA16A
SNORA21
SNORA23
SNORA24
SNORA33
SNORA34
SNORA38
SNORA49
SNORA50
SNORA52
SNORA57
SNORA6
SNORA62
SNORA63
SNORA65
SNORA67
SNORA68
SNORA71A
SNORA71B
SNORA71C
SNORA71D
SNORA74A
SNORA74B
SNORA7B
SNORA84
SNORA9
SNORD15A
SNORD15B
SNORD17
SNORD94
SNRPD1
TABLE 1-18
SNRPE
SNRPF
SNRPG
SOS1
SPINK5
SPINK7
SPRED1
SPRRIA
SPRRIB
SPRR2D
SPRR2E
SPRR2F
SPRR3
SPTLC1
SPTLC2
SRD5A1
SRSF10
SSBP1
SSBP3
STAP2
SUMF2
SYBU
TADA2B
TCEAl
TCHH
TCHHL1
TFAP2C
TFIP11
TGM3
THOC7
TIA1
TM4SF1
TM4SF19
TMEM179B
TMEM45B
TMEM60
TPRG1
TRAF4
TRAK2
TRAPPC2L
TRMT6
TABLE 1-19
TRPT1
TSC2
TSPO
TSR1
TTPAL
TUBB2A
TWF1
TXNDC17
TXNRD1
UBE2L3
UBL3
UBL5
UCHL3
UGP2
UNC50
UQCR10
UQCRH
UTP6
VASN
VPS4A
VSIG8
WDR60
WDR61
WFDC12
WFDC5
WIBG
WWTR1
XPOT
YTHDF1
YTHDF2
ZFAND2A
ZNF259
TABLE 1-20
ABTB1
ADAM8
ADORA2A
AGTRAP
AGXT2L2
AHCYL1
ALPL
ANKRD12
ANKRD17
ANKRD27
AP1G1
APH1A
ARF1
ARF5
ARHGAP30
ARHGEF2
ARID3A
ARL5B
ARPC2
ATG2A
ATHL1
ATP13A3
ATP6V0C
ATP6V0D1
AURKAIP1
BAK1
BAP1
BMP2K
BRD2
BSDC1
C15orf38
C17orfl07
C22orfl3
CAMKID
CANT1
CASP9
CCDC28A
CCDC9
CCL3
CCNI
CCRL2
TABLE 1-21
CD63
CD83
CD97
CDC42SE1
CDKN1A
CFL1
CHD2
CIC
CNN2
CRLF3
CSF1
CSF2RB
CSRNP1
CTBP2
CTDSP2
CXCR4
CYTH1
DBNL
DCAF11
DENND5A
DESI1
DGAT1
DNM2
DOTIL
DUSP1
DUSP2
DUSP3
ECD
EFHD2
EFR3A
EGR2
EGR3
EIF2C4
EIF4EBP2
ELF1
EMP3
EPS15L1
FAM100B
FAM193B
FAM210A
FAM32A
TABLE 1-22
FAM53C
FBXO11
FCGRT
FGR
FLNA
FNIP1
FOSB
FOSL2
FOXN3
FOXO4
FURIN
FZR1
GABARAPL1
GADD45B
GAPVD1
GATAD2A
GGA1
GLA
GMIP
GNB1
GNB2
GPR108
GPX1
GRAMD1A
GRK6
GRN
GTPBP1
HEXIM1
HIPK3
HLA.A
HLX
HSPA4
IDS
IER3
IMPDH1
INO80D
INPP5K
IQSEC1
IRAK2
IRS2
ISCU
TABLE 1-23
ISG20L2
ITGA5
ITGAM
ITGAX
JARID2
JUNB
KAT5
KDM6B
KIAA0232
KIAA0513
KLF2
KLF6
KLHL2
LATS2
LILRB2
LIMS1
LITAF
LOC283070
LPAR2
LPCAT1
LSP1
LTBR
MAFI
MAN2A1
MAP4K4
MAP7D1
MAPKAPK2
MECP2
MEF2D
METRNL
MGEA5
MIDN
MKNK2
MLF2
MLLT6
MMP25
MTHFS
MTMR14
MYADM
MY09B
NAA50
TABLE 1-24
NAB1
NAGK
NCF1B
NCF1C
NCOA1
NFKB2
NFKBIB
NFKBID
NINJ1
NLRC5
NOTCH2NL
NRIP1
NUMB
OGFR
OS9
PAN3
PATL1
PCBP1
PDPK1
PER1
PFKFB3
PHF1
PIK3AP1
PIK3R5
PIM3
PITPNA
PLAU
PLEKHB2
PLEKHM3
PLIN5
PPP1R15A
PPP1R18
PPP2R5C
PPP4R1
PRR14
PRR24
PRRC2C
PTGER4
PTK2B
PTTG1IP
RAB11FIP1
TABLE 1-25
RAB20
RAB5C
RALGDS
RAP2C
RBCK1
RBM39
RBM4
RELA
RGS19
RHBDD2
RHEB
RHOA
RHOB
RILPL2
RNASEK
RNF13
RNF41
RTN4
RXRA
RYBP
SBNO2
SCYL1
SDE2
SEC22B
SEMA6B
SERINCI
SERP1
SF3B2
SH3BP5
SHISA5
SIPA1
SIRPA
SLC11A1
SLC15A3
SLC16A3
SLC25A6
SLC3A2
SLC43A2
SLC44A2
SLC6A6
SLC9A8
TABLE 1-26
SLED1
SMG1P1
SPHK1
SQSTM1
SREBF2
SRRM2
SRXN1
STK40
STX11
STX3
STX6
STXBP2
SUPT6H
TAF10
TANK
TCF25
TCIRG1
TM9SF4
TMBIM6
TMEM123
TMEM167B
TMEM183A
TMEM66
TMX4
TNFAIP2
TNFAIP3
TNFRSF14
TOM1
TP53INP2
TRAPPC5
TSPAN13
TTYH3
UBAP2L
UBE2D3
UBR4
UCP2
UPF1
USB1
USF2
WBP2
WDR82
TABLE 1-27
XPO6
YPEL5
ZC3H12A
ZFP36
ZMIZ1
ZNFX1
ZZEF1
TABLE 4-1
ACOT2
ACOX3
ACTG1
AKT1S1
AMZ2
ANXA1
ANXA2
AQP3
AREG
ARF5
ATP5E
BCKDK
BCR
BSG
C14orf2
CEBPA
CHCHD2
CHMP5
COPE
COROIA
CSDA
DYNLT1
EIF4A3
EMP1
FLII
GPR157
GPX3
HSPA1A
KRT16
LOC100216546
LOC100288069
MESDC1
MIEN1
MKNK2
MNDA
NEDD8
OTUD1
PIR
PNISR
POLR2J3
TABLE 4-2
PQLC1
PRELID1
PRKAA1
PSMA7
PSMD4
PTGS2
RASAL1
RNASET2
RNF217
RPL13
S100A8
SDC4
SERPINB4
SLC25A3
SLPI
SNORA24
SNORA50
SNORA57
SNORA8
SNORA9
SOCS3
TIMP1
TMCC3
TRMT44
TSPO
TUBA1C
UQCRB
UQCRC1
UQCRFS1
VEGFA
ZFP36L2
ZNF410
ZSWIM6
TABLE 4-3
AATF
ADRBK2
AHSA1
AIDA
ANKRD12
ANXA3
AP3B1
APH1A
API5
APLP2
ARID4B
ARPCIA
ARPC3
ATG12
ATP2A2
ATP5J2
ATP6AP2
ATP6VOC
ATP6V1G1
BAG1
BHLHE40
BTF3
BTG1
BUD31
C14orf178
CAPZA1
CAPZA2
CBFB
CCDC93
CCL3
CCNI
CDC42
CHMP2A
CHMP2B
CHMP3
CIRBP
CLIC4
CLIP1
CLK1
CLNS1A
CNBP
TABLE 4-4
COPB2
CPA4
CPM
CS
CSF1
CXCR4
CYBB
DCUN1D1
DDX21
DDX5
DICER1
DLD
DNAJC15
DNAJC3
DR1
EEF1B2
EGR2
EIF2S2
EIF5A
ELF1
EML4
EP300
EPS15
ERBB2IP
ETF1
ETV6
EVL
EZR
FAM100A
FAM126A
FAM160A1
FNTA
FUBP1
FYTTD1
G3BP2
GABARAP
GABARAPL1
GLTP
GLTSCR2
GOLGA8B
GRB2
TABLE 4-5
HBP1
HELZ
HIF1A
HINT1
HINT3
HIST1H1E
HMGN1
HNRNPA2B1
HNRNPK
HNRNPU
IARS2
ICAM1
IDE
IER3IP1
JAK1
JMY
KAT2B
KIAA1551
KIF16B
KLF10
KLF3
LGALSL
MARCH7
MBD2
MBD6
MDM2
MED13L
MED19
MRPL15
NAPA
NR4A2
NRBF2
NRBP1
NSFP1
0GFRL1
P4HB
PAIP2
PDXK
PGK1
PGRMC2
PHF20L1
TABLE 4-6
PHF5A
PIKFYVE
PLA2G7
POLR2A
PTPN12
QARS
RAB14
RAB9A
RABGEF1
RAP1A
RAP1B
RHOA
RIOK3
RMND5A
RNASEK
RPL10
RPL13AP20
RPL15
RPL19
RPL24
RPL26
RPL28
RPL36AL
RPL5
RPL6
RPS20
RPS25
RPS9
S100A10
S100A11
SCAF11
SCYL2
SDF4
SEC11C
SEC24A
SEPT11
SEPT2
SERINCI
SERINC3
SERPINA12
SERPINB9
TABLE 4-7
SERTAD2
SET
SH3BGRL3
SLMO2
SMS
SNAP29
SNORA53
SNX13
SNX9
SREK1IP1
SRSF5
SSR2
SSU72
STK24
STT3B
TAF10
TAOK1
TERF2IP
TLK2
TMA7
TMEM106B
TMEM127
TMEM167B
TNFSF13B
TPGS2
TRAM1
TRIP12
TRPM7
TSG101
TXNL1
UBE2A
UBE2B
UBE2H
USMG5
USP22
USP53
USP6NL
USP7
WIPF1
WTAP
XBP1
TABLE 4-8
YWHAQ
ZCRB1
ZMAT2
ZNF148
TABLE 4-9
ALOX12B
ANXA1
AQP3
ATP12A
ATP5B
ATP5I
ATP5O
BAG3
C6orf132
CALM1
CASP14
CAST
CDSN
CLIC3
CNFN
COX4I1
COX8A
CRABP2
CST6
CTSC
DNAJA1
DYNLL1
EEF1B2
EIF1AX
EIF3K
ELF3
EMP1
EPHX3
FABP9
GNB2L1
GRHL3
HIST1H4E
HIST1H4H
HMGCS1
HMOX2
HSP90AA1
HSPB1
IVL
KLF5
KLK13
KLK7
TABLE 4-10
KRT10
KRT14
KRT16
KRT25
KRT27
KRT5
KRT6A
KRT71
KRT72
KRT74
KRTAP5-3
KRTDAP
LCE2C
LCE2D
LCE3D
LCE3E
LCN2
LNX1
LRRC15
NDRG2
NDUFA4L2
NDUFB11
NDUFB2
NDUFB8
NDUFS5
NSFL1C
NUMA1
PDZK1IP1
PINLYP
PKP1
PNP
POLR2L
PPL
PPP2R2A
PRR9
PRSS3
PSMC2
RBBP4
RMRP
ROMO1
RPL10A
TABLE 4-11
RPL11
RPL12
RPL13A
RPL18
RPL21
RPL26
RPL27
RPL27A
RPL29
RPL3
RPL30
RPL32
RPL35
RPL36
RPL36A
RPL37A
RPL38
RPL7
RPL7A
RPLP0
RPLP1
RPLP2
RPS10
RPS12
RPS15
RPS15A
RPS18
RPS19
RPS21
RPS26
RPS28
RPS3
RPS4X
RPS5
RPS6
RPS8
S100A14
S100A7
S100A7A
S100A9
SBDS
TABLE 4-12
SBSN
SERPINB4
SERPINB5
SFN
SLURP1
SNORA16A
SNORA24
SNORA52
SNORA63
SNORA68
SNORA71A
SNORD15B
SPRR1A
SPRR1B
SPRR2D
SPRR2E
SPRR2F
TCHH
TCHHL1
TMOD3
TMPRSS11E
UBE2L3
UBL3
UQCR11
UQCRH
UXT
WWC1
WWTR1
TABLE 4-13
A2M
AADACL3
ABHD5
ABTB1
ACSL5
ADAM8
ADORA2A
AGTRAP
AKR7A2
ALPL
AMPD2
ANKRD22
AP5B1
ARF1
ARF5
ARHGAP1
ARHGAP30
ARHGEF2
ARID3A
ARL5B
ARRB2
ASAH1
ATG2A
ATHL1
ATP6V0C
BASP1
BCKDK
BCL2L1
BHLHE40
BRD4
C17orf107
C1orf43
C22orf13
C2CD2
C6orf106
CANT1
CCDC86
CCL3
CCL3L3
CCL4
CCNI
TABLE 4-14
CCNY
CCRL2
CCSAP
CD300A
CD36
CD63
CD82
CD83
CD97
CDC14A
CDC37
CDC42EP3
CDC42SE1
CDKN1A
CEP76
CHD2
CHMP4B
CHP1
CLMP
CNN2
COTL1
CRKL
CSF2RB
CSF3R
CSNK1G2
CSRNP1
CTSA
CTSD
CXCL16
CXCR4
CYTH4
DBNL
DCAF11
DDX60L
DENND5A
DGAT2
DHCR24
DIRC2
DSCR3
DUSP1
DUSP2
TABLE 4-15
DUSP3
DUSP4
ECE1
EFHD2
EFR3A
EGR2
EGR3
EHBP1L1
EHD1
EID3
EIF1
EIF4EBP2
EIF4EBP3
ELL
EMP3
EPS15L1
FADS2
FAM100B
FAM193B
FAM213A
FAM32A
FAM46C
FFAR2
FGR
FLNA
FMNL1
FNIP1
FOSB
FOSL2
FURIN
GABARAPL1
GADD45B
GAL
GAS7
GDE1
GPR108
GPR157
GPSM3
GRAMD1A
GRINA
GRK6
TABLE 4-16
GRN
GTPBP1
HDAC7
HLA-A
HPCAL1
HS3ST6
HSPA4
IDS
IER3
IMPDH1
INPP5K
IRAK2
IRF1
ITGA5
ITGAX
ITPK1
JUNB
KIAA0247
KIAA0368
KIAA0494
KIAA1191
KLF2
KLF6
LARP1
LGALS3
LILRB2
LILRB3
LIMK2
LITAF
LOC146880
LOC729737
LPCAT1
LPIN1
LSP1
LTBR
MAF1
MAP4K4
MAP7D1
MAPKAPK2
MARCKS
MBOAT7
TABLE 4-17
MEF2D
MEGF9
MEPCE
METRNL
MGEA5
MKNK2
MLF2
MLLT6
MMP25
MSRB1
MTHFS
MTMR14
MYO9B
NAA50
NBEAL2
NCF1B
NFKB2
NFKBIA
NFKBIB
NFKBID
NFKBIE
NINJ1
NIPBL
NLRC5
NOTCH2NL
NR4A3
NTAN1
OGDH
OSM
P2RY4
PACSIN2
PDHX
PDLIM7
PER1
PFKL
PHF1
PIK3AP1
PIK3R5
PILRA
PIM2
PIM3
TABLE 4-18
PITPNA
PLAU
PLEKHO2
POU5F1P3
PPPICB
PPP1R15A
PPP1R18
PPP4R1
PSMF1
PTGER4
PTK2B
PTPN6
PTTG1IP
RAB11FIP1
RAB20
RAB27A
RAB5B
RAB5C
RALGDS
RANGAP1
RAP2A
RBCK1
RBM39
RELA
RHEB
RHOA
RHOB
RILPL2
RIT1
RNASEK
RNF213
RTN4
RXRA
RYBP
SBNO2
SCARF1
SCD
SCYL1
SERINC1
SH2B2
SH3BP5
TABLE 4-19
SHISA5
SHKBP1
SIRPA
SLC11A1
SLC15A3
SLC15A4
SLC31A1
SLC3A2
SLC41A1
SLC43A2
SLC43A3
SLC45A4
SLC6A6
SMG1P1
SNORA8
SORT1
SPHK1
SPINT2
SQSTM1
SREBF2
SRP54
SRRM2
SRXN1
STK40
STX11
STX6
STXBP2
TAGAP
TAP1
TCF25
TCIRG1
TECPR2
TEX264
TLE3
TMBIM6
TMEM123
TMEM134
TMEM189
TNFAIP2
TNFRSF14
TNIP1
TABLE 4-20
TOM1
TPD52L2
TRIB1
TRIM25
TRPC4AP
UBAP2L
UBE2D3
UBIAD1
UBR4
UCP2
USF2
VOPP1
WBP2
WSB2
XPO6
YKT6
ZC3H12A
ZFP36
ZFP36L1
ZHX2
ZMIZ1

11.-13. (canceled)
